(19) United States
(12) Patent Application Publication
(10) Pub. No.: US 2024/0287504 A1
(43) Pub. Date: Aug. 29, 2024

(54) GENOMIC LIBRARY PREPARATION AND TARGETED EPIGENETIC ASSAYS USING CAS-GRNA RIBONUCLEOPROTEINS

(71) Applicants: Illumina, Inc., San Diego, CA (US); Illumina Cambridge Limited, Cambridge (GB)

(72) Inventors: Andrew Kennedy, San Diego, CA (US); Sarah Shultzaberger, San Diego, CA (US); Emma Bell, Sawston (GB); Oliver Miller, Linton (GB); Kim Schneider, Cambridge (GB); Esther Musgrave-Brown, Cambridge (GB); Niall Gormley, Cambridge (GB); Andrew Slatter, Cambridge (GB); Feng Chen, Castro Valley, CA (US)

(73) Assignees: Illumina, Inc., San Diego, CA (US); Illumina Cambridge Limited, Cambridge (GB)

(21) Appl. No.: 18/549,344

(22) PCT Filed: Mar. 8, 2022
(86) PCT No.: PCT/US22/19252

§ 371 Date: Sep. 9, 2023

Related U.S. Application Data
(60) Provisional application No. 63/158,492, filed on Mar. 9, 2021.
(60) Provisional application No. 63/162,775, filed on Mar. 18, 2021.
(60) Provisional application No. 63/163,381, filed on Mar. 19, 2021.
(60) Provisional application No. 63/228,344, filed on Aug. 2, 2021.
(60) Provisional application No. 63/246,879, filed on Sep. 22, 2021.
(60) Provisional application No. 63/295,432, filed on Dec. 30, 2021.

(51) Int. Cl. C12N 15/10

(52) U.S. Cl. CPC C12N 15/1065

(57) ABSTRACT

Genomic library preparation using Cas-gRNA RNPs, and targeted epigenetic assays, are provided herein. Some compositions include, from a first species, substantially only single-stranded polynucleotides; from a second species, substantially only double-stranded polynucleotides; and amplification primers ligated to ends of the second double-stranded polynucleotides and substantially not ligated to any ends of the first double-stranded polynucleotides. Some compositions include first and second molecules of a target polynucleotide having a sequence, the first molecule having a first end at a first subsequence, the second molecule having a first end at a second subsequence, wherein the first subsequence only partially overlaps with the second subsequence. Some examples provide a composition that includes a target polynucleotide and a first fusion protein including a Cas-gRNA RNP coupled to a transposase having an amplification adapter coupled thereto. The Cas-gRNA RNP may be hybridized to a subsequence in the target polynucleotide.

### CROSS-REFERENCE TO RELATED APPLICATIONS

This application claims the benefit of the following applications, the entire contents of each of which are incorporated by reference herein:
U.S. Provisional Patent Application No. 63/158,492, filed Mar. 9, 2021 and entitled “Genomic library preparation and targeted epigenetic assays using Cas-gRNA ribonucleoproteins;”
U.S. Provisional Patent Application No. 63/162,775, filed Mar. 18, 2021 and entitled “Genomic library preparation and targeted epigenetic assays using Cas-gRNA ribonucleoproteins;”
U.S. Provisional Patent Application No. 63/163,381, filed Mar. 19, 2021 and entitled “Genomic library preparation and targeted epigenetic assays using Cas-gRNA ribonucleoproteins;”
U.S. Provisional Patent Application No. 63/228,344, filed Aug. 2, 2021 and entitled “Genomic library preparation and targeted epigenetic assays using Cas-gRNA ribonucleoproteins;”
U.S. Provisional Patent Application No. 63/246,879, filed Sep. 22, 2021 and entitled “Genomic library preparation and targeted epigenetic assays using Cas-gRNA ribonucleoproteins;” and
U.S. Provisional Patent Application No. 63/295,432, filed Dec. 30, 2021 and entitled “Genomic library preparation and targeting epigenetic assays using Cas-gRNA ribonucleoproteins.”

### FIELD

This application relates to compositions and methods that use Cas-gRNA RNPs for genomic library preparation and targeted epigenetic assays.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is named 8549102416_SL.txt. The text file is about 1.29 KB, was created on Mar. 3, 2022, and is being submitted electronically via EFS-Web.

### BACKGROUND

Clustered regularly interspaced short palindromic repeats (CRISPRs) are involved in an interference pathway that protects cells from bacteriophages and conjugative plasmids in many bacteria and archaea; see, e.g., Marraffini et al., “CRISPR interference: RNA-directed adaptive immunity in bacteria and archaea,” Nat Rev Genet. 11(3): 181-190 (2010), the entire contents of which are incorporated by reference herein. CRISPR sequences include arrays of short repeat sequences that are interspaced by unique variable DNA sequences of similar size called spacers, which often originate from phage or plasmid DNA; see, e.g., the following references, the entire contents of which are incorporated by reference herein: Barrangou et al., “CRISPR provides acquired resistances against viruses in prokaryotes,” Science 315:1709-1712 (2007); Bolotin et al., “Clustered regularly interspersed short palindrome repeats (CRISPRs) have spacers of extrachromosomal origin,” Microbiology 151:2551-1561 (2005); and Mojica et al., “Intervening sequences of regularly spaced prokaryotic repeats derive from foreign genetic elements,” J Mol Evol. 60:174-82 (2005). Thus, CRISPR sequences provide an adaptive, heritable record of past infections and may be transcribed into CRISPR RNAs (crRNAs)-small RNAs that target invasive polynucleotides (see, e.g., Marraffini et al., cited above). CRISPRs are often associated with CRISPR-associated (Cas) genes that code for proteins related to CRISPRs. Cas proteins can provide mechanisms for destroying invading foreign polynucleotides targeted by crRNAs. CRISPRs together with Cas genes provide an adaptive immune system that provides acquired resistance against invading foreign polynucleotides in bacteria and archaea (see, e.g., Barrangou et al., cited above).

Single-molecule sequencing studies have suggested CRISPR-targeted methods for direct methylation sequencing with Cas9; see, e.g., Gilpatrick et al., “Targeted nanopore sequencing with Cas9 for studies of methylation, structural variants and mutations,” https://doi.org/10.1101/604173, 1-14 (2019), the entire contents of which are incorporated by reference herein. Beyond DNA methylation, however, there is an unmet need for methods enabling sensitive characterization of epigenetic changes at targeted DNA loci. Chromatin accessibility (by ATAC-seq) and protein(s) associated with a DNA locus (by ChIP-seq) are examples of epigenetic elements that are difficult to target with existing hybrid capture technology. Commonly, assays that enrich for DNA sequences are associated with an epigenetic feature. However, as these sequences are not known a priori, it is challenging to design appropriate hybrid capture oligonucleotides to efficiently enrich the output of the epigenetic assay for a particular genomic region of interest (e.g., a genomic locus).

Prior methods of using deactivated Cas (dCas9) for targeted locus-specific protein isolation to identify histone gene regulators have been presented; see, e.g., Tsui et al., “dCas9-targeted locus-specific protein isolation method identifies histone gene regulators,” PNAS 115(2): E2734-E2741 (2018), the entire contents of which are incorporated by reference herein. Such methods demonstrated that dCas9-based locus enrichment can isolate chromatin that can be subsequently assayed by mass spectrometry. However, this method only allows a single chromatin locus to be assayed in each experiment. Furthermore, this prior work provides two separate results, i.e. the sequence of the DNA locus, and mass spectrometry to identify DNA associated proteins. Improved methods for locus-targeted epigenetic analysis are needed.

### SUMMARY

Genomic library preparation, and targeted epigenetic assays, using Cas-gRNA ribonucleoproteins (RNPs), are provided herein.

Some examples herein provide a method of treating a mixture of first double-stranded polynucleotides from a first species and second double-stranded polynucleotides from a second species, The method may include protecting ends of the first double-stranded polynucleotides and any ends of the second double-stranded polynucleotides. The method may include, after protecting the ends of the first and second double-stranded polynucleotides, selectively generating free ends within the first double-stranded polynucleotides. The method may include degrading the first double-stranded polynucleotides from the free ends toward the protected ends.

In some examples, selectively generating the free ends within the first double-stranded polynucleotides includes hybridizing CRISPR-associated protein guide RNA ribonucleoproteins (Cas-gRNA RNPs) to sequences that are present within the first double-stranded polynucleotides and that are not present within the second double-stranded polynucleotides, and cutting the sequences with the Cas-gRNA RNPs. In some examples, the sequences include mammalian specific repetitive elements. In some examples, the mammalian specific repetitive elements include human specific repetitive elements. In some examples, the second species is bacterial, fungal, or viral. In some examples, the first double-stranded nucleotides comprise a plurality of chromosomes from the first species.

In some examples, protecting ends of the first and second double-stranded polynucleotides includes ligating hairpin adapters to the ends. In some examples, protecting ends of the first and second double-stranded polynucleotides includes 5′-dephosphorylating the ends. In some examples, protecting ends of the first and second double-stranded polynucleotides includes adding modified bases to the ends. In some examples, the modified bases include phosphorothioate bonds. In some examples, the modified bases are added using a terminal transferase.

In some examples, degrading the first double-stranded polynucleotides is performed using an exonuclease.

In some examples, the free ends include 3′ ends. In some examples, degrading the first double-stranded polynucleotides is performed using exonuclease III. In some examples, the free ends include 5′ ends. In some examples, degrading the first double-stranded polynucleotides is performed using Lambda exonuclease.

In some examples, the method further includes subsequently ligating amplification adapters to the ends of any remaining double-stranded polynucleotides in the mixture. In some examples, the amplification adapters include unique molecular identifiers (UMIs). In some examples, the method further includes subsequently amplifying and sequencing the double-stranded polynucleotides.

In some examples, the first double-stranded polynucleotides include double-stranded DNA. In some examples, the second double-stranded polynucleotides include double-stranded DNA. In some examples, the second double-stranded polynucleotides include circular DNA.

In some examples, the Cas includes Cas9.

Some examples herein provide a composition. The composition may include first double-stranded polynucleotides from a first species. Ends of the first double-stranded polynucleotides may be protected. The composition may include second double-stranded polynucleotides from a second species. Any ends of the second double-stranded polynucleotides may be protected. The composition also may include CRISPR-associated protein guide RNA ribonucleoproteins (Cas-gRNA RNPs) hybridized to sequences that are present within the first double-stranded polynucleotides and that are not present within the second double-stranded polynucleotides. The Cas-gRNA RNPs may be for cutting the sequences so as to selectively generate free ends within the first double-stranded polynucleotides.

In some examples, the sequences include mammalian specific repetitive elements. In some examples, the mammalian specific repetitive elements include human repetitive elements. In some examples, the second species is bacterial, fungal, or viral.

In some examples, the ends of the first and second double-stranded polynucleotides are protected using hairpin adapters. In some examples, the ends of first and second double-stranded polynucleotides are protected using 5′-dephosphorylation. In some examples, the ends of the first and second double-stranded polynucleotides are protected using modified bases. In some examples, the modified bases include phosphorothioate bonds.

In some examples, the free ends include 3′ ends. In some examples, the free ends include 5′ ends.

In some examples, the first double-stranded polynucleotides include double-stranded DNA. In some examples, the second double-stranded polynucleotides include double-stranded DNA. In some examples, the second double-stranded polynucleotides include circular DNA.

In some examples, the Cas includes Cas9.

Some examples herein provide a method of treating a mixture of first double-stranded polynucleotides from a first species and second double-stranded polynucleotides from a second species. The method may include selectively making the first double-stranded polynucleotides in the mixture single-stranded. The method may include subsequently selectively ligating amplification primers to any remaining double-stranded polynucleotides in the mixture. The method may include subsequently amplifying any double-stranded polynucleotides in the mixture to which amplification primers were ligated.

Some examples herein provide a composition. The composition may include, from a first species, substantially only single-stranded polynucleotides. The composition may include, from a second species, substantially only double-stranded polynucleotides. The composition may include amplification primers ligated to ends of the second double-stranded polynucleotides and substantially not ligated to any ends of the first double-stranded polynucleotides.

Some examples herein provide a method of generating fragments of a whole genome (WG). The method may include, within a first sample of the WG, hybridizing a first set of CRISPR-associated protein guide RNA ribonucleoproteins (Cas-gRNA RNPs) to first sequences in the WG that are spaced apart from one another by approximately a first number of base pairs. The method further may include, within the first sample of the WG, hybridizing a second set of Cas-gRNA RNPs to second sequences in the WG that are spaced apart from one another by approximately a second number of base pairs. The method further may include, within the first sample of the WG, respectively cutting the first and second sequences with the first and second sets of Cas-gRNA RNPs in the first sample to generate a first set of WG fragments each having approximately the same number of base pairs as one another.

In some examples, the first number of base pairs is approximately the same as the second number of base pairs. In some examples, the first number of base pairs is between about 100 and about 2000, and the second number of base pairs is between about 100 and about 2000. In some examples, the first number of base pairs is between about 500 and about 700, and the second number of base pairs is between about 500 and about 700. In some examples, the number of base pairs in the WG fragments of the first set of WG fragments varies by less than about 20%.

In some examples, the method further includes, within a second sample of the WG, hybridizing the first set of Cas-gRNA RNPs to the first sequences in the WG. The method further may include, within the second sample of the WG, hybridizing the second set of Cas-gRNA RNPs to the second sequences in the WG. The method further may include, within the second sample of the WG, hybridizing a third set of Cas-gRNA RNPs to third sequences in the WG that are spaced apart from one another by approximately a third number of base pairs. The method further may include, within the second sample of the WG, respectively cutting the first, second, and third sequences with the first, second, and third sets of Cas-gRNA RNPs to generate a second set of WG fragments each having approximately the same number of base pairs as one another.

In some examples, the third number of base pairs is different than the first number of base pairs. In some examples, the third number of base pairs is different than the second number of base pairs. In some examples, the third number of base pairs is between about 100 and about 2000. In some examples, the third number of base pairs is between about 200 and about 400. In some examples, the approximate number of base pairs in the WG fragments of the second set of WG fragments is different than the approximate number of base pairs in the WG fragments of the first set of WG fragments. In some examples, the number of base pairs in the WG fragments of the second set of WG fragments varies by less than about 20%.

In some examples, the method further includes, within a third sample of the WG, respectively hybridizing the first, second, or third set of Cas-gRNA RNPs to the first, second, or third sequences in the WG. The method further may include respectively cutting the first, second, or third sequences with the first, second, or third set of Cas-gRNA RNPs to generate a third set of WG fragments each having approximately the same number of base pairs as one another.

In some examples, the approximate number of base pairs in the WG fragments of the third set of WG fragments is different than the approximate number of base pairs in the WG fragments of the first set of WG fragments. In some examples, the approximate number of base pairs in the WG fragments of the third set of WG fragments is different than the approximate number of base pairs in the WG fragments of the second set of WG fragments. In some examples, the number of base pairs in the WG fragments of the third set of WG fragments varies by less than about 20%.

In some examples, the method further includes ligating amplification adapters to ends of the WG fragments of the third set of WG fragments. The method further may include generating amplicons of the WG fragments of the third set of WG fragments having the amplification adapters ligated thereto. The method further may include sequencing the amplicons of the WG fragments of the third set of WG fragments. In some examples, amplicons of the WG fragments of the second and third sets of WG fragments are mixed together for the sequencing. In some examples, amplicons of the WG fragments of the first and third sets of WG fragments are mixed together for the amplification and sequencing.

In some examples, the number of base pairs in the WG fragments of the third set of WG fragments is between about 100 and about 1000. In some examples, the number of base pairs in the WG fragments of the third set of WG fragments is between about 500 and about 700.

In some examples, the third set of Cas-gRNA RNPs includes at least about 1,000,000 different Cas-gRNA RNPs.

In some examples, the method further includes ligating amplification adapters to ends of the WG fragments of the second set of WG fragments. The method further may include generating amplicons of the WG fragments of the second set of WG fragments having the amplification adapters ligated thereto. The method further may include sequencing the amplicons of the WG fragments of the second set of WG fragments.

In some examples, amplicons of the WG fragments of the first and second sets of WG fragments are mixed together for the amplification and sequencing.

In some examples, the number of base pairs in the WG fragments of the second set of WG fragments is between about 100 and about 1000. In some examples, the number of base pairs in the WG fragments of the second set of WG fragments is between about 100 and about 200.

In some examples, the method further includes ligating amplification adapters to ends of the WG fragments of the first set of WG fragments. The method further may include generating amplicons of the WG fragments of the first set of WG fragments having the amplification adapters ligated thereto. The method further may include sequencing the amplicons of the WG fragments of the first set of WG fragments.

In some examples, the amplification adapters include unique molecular identifiers (UMIs).

In some examples, the number of base pairs in the WG fragments of the first set of WG fragments is between about 100 and about 1000. In some examples, the number of base pairs in the WG fragments of the first set of WG fragments is between about 200 and about 400.

In some examples, the first set of Cas-gRNA RNPs includes at least about 1,000,000 different Cas-gRNA RNPs. In some examples, the second set of Cas-gRNA RNPs includes at least about 1,000,000 different Cas-gRNA RNPs.

In some examples, the WG includes double-stranded DNA. In some examples, the Cas includes Cas9.

Some examples herein provide a composition. The composition may include a sample of a whole genome (WG). The composition may include a first set of CRISPR-associated protein guide RNA ribonucleoproteins (Cas-gRNA RNPs) hybridized to first sequences in the WG that are spaced apart from one another by approximately a first number of base pairs. The composition may include a second set of Cas-gRNA RNPs hybridized to second sequences in the WG that are spaced apart from one another by approximately a second number of base pairs. The first and second sets of Cas-gRNA RNPs respectively may be for cutting the first and second sequences within the sample to generate WG fragments each having approximately the same number of base pairs as one another.

In some examples, the first number of base pairs is approximately the same as the second number of base pairs. In some examples, the first number of base pairs is between about 100 and about 2000, and the second number of base pairs is between about 100 and about 2000. In some examples, the first number of base pairs is between about 500 and about 700, and the second number of base pairs is between about 500 and about 700.

In some examples, the number of base pairs in the WG fragments varies by less than about 20%. In some examples, the number of base pairs in the WG fragments is between about 100 base pairs and about 1000 base pairs. In some examples, the number of base pairs in the WG fragments is between about 200 base pairs and about 400 base pairs.

In some examples, the first set of Cas-gRNA RNPs includes at least about 1,000,000 different Cas-gRNA RNPs. In some examples, the second set of Cas-gRNA RNPs includes at least about 1,000,000 different Cas-gRNA RNPs.

In some examples, the WG includes double-stranded DNA. In some examples, the Cas includes Cas9.

Some examples herein provide a composition. The composition may include a sample of a whole genome (WG). The composition may include a first set of CRISPR-associated protein guide RNA ribonucleoproteins (Cas-gRNA RNPs) hybridized to first sequences in the WG that are spaced apart from one another by approximately a first number of base pairs. The composition may include a second set of Cas-gRNA RNPs hybridized to second sequences in the WG that are spaced apart from one another by approximately a second number of base pairs. The composition may include a third set of Cas-gRNA RNPs hybridized to third sequences in the WG that are spaced apart from one another by approximately a third number of base pairs. The first, second, and third sets of Cas-gRNA RNPs respectively may be for cutting the first, second, and third sequences within the sample to generate WG fragments each having approximately the same number of base pairs as one another.

In some examples, the first number of base pairs is approximately the same as the second number of base pairs. In some examples, the first number of base pairs is between about 100 and about 2000, the second number of base pairs is between about 100 and about 2000, and the third number of base pairs is between about 100 and about 2000. In some examples, the first number of base pairs is between about 500 and about 700, the second number of base pairs is between about 500 and about 700, and the third number of base pairs is between about 200 and about 400. In some examples, the third number of base pairs is different than the first number of base pairs. In some examples, the third number of base pairs is different than the second number of base pairs.

In some examples, the number of base pairs in the WG fragments varies by less than about 20%. In some examples, the number of base pairs in the WG fragments is between about 100 and about 1000. In some examples, the number of base pairs in the WG fragments is between about 100 and about 200.

In some examples, the first set of Cas-gRNA RNPs includes at least about 1,000,000 different Cas-gRNA RNPs. In some examples, the second set of Cas-gRNA RNPs includes at least about 1,000,000 different Cas-gRNA RNPs. In some examples, the third set of Cas-gRNA RNPs includes at least about 1,000,000 different Cas-gRNA RNPs.

In some examples, the WG includes double-stranded DNA. In some examples, the Cas includes Cas9.

Some examples herein provide a method of generating fragments of a whole genome (WG). The method may include hybridizing a set of CRISPR-associated protein guide RNA ribonucleoproteins (Cas-gRNA RNPs) to sequences in the WG that are spaced apart from one another by approximately a number of base pairs. The method may include respectively cutting the sequences with the set of Cas-gRNA RNPs to generate a set of WG fragments each having approximately the same number of base pairs as one another.

In some examples, the number of base pairs is between about 100 and about 1000. In some examples, the number of base pairs is between about 500 and about 700, or between about 200 and about 400, or between about 100 and about 200.

In some examples, the number of base pairs in the WG fragments of the set of WG fragments varies by less than about 20%. In some examples, the number of base pairs in the WG fragments of the set of WG fragments is between about 100 and about 1000. In some examples, the number of base pairs in the WG fragments of the set of WG fragments is between about 100 and about 200, or between about 200 and about 400, or between about 500 and about 700.

In some examples, the method further includes ligating amplification adapters to ends of the WG fragments of the set of WG fragments. The method further may include generating amplicons of the WG fragments of the set of WG fragments having the amplification adapters ligated thereto. The method further may include sequencing the amplicons of the WG fragments of the set of WG fragments.

In some examples, the amplification adapters include unique molecular identifiers (UMIs).

In some examples, the WG includes double-stranded DNA. In some examples, the Cas includes Cas9.

Some examples herein provide a composition. The composition may include a sample of a whole genome (WG). The composition may include a set of CRISPR-associated protein guide RNA ribonucleoproteins (Cas-gRNA RNPs) hybridized to sequences in the WG that are spaced apart from one another by approximately a number of base pairs. The set of Cas-gRNA RNPs respectively may be for cutting the sequences within the sample to generate WG fragments each having approximately the same number of base pairs as one another.

In some examples, the number of base pairs is between about 100 and about 1000. In some examples, the number of base pairs is between about 500 and about 700, or between about 200 and about 400, or between about 100 and about 200.

In some examples, the number of base pairs in the WG fragments of the set of WG fragments varies by less than about 20%. In some examples, the number of base pairs in the WG fragments of the set of WG fragments is between about 100 and about 1000. In some examples, the number of base pairs in the WG fragments of the set of WG fragments is between about 100 and about 200, or between about 200 and about 400, or between about 500 and about 700.

In some examples, the WG includes double-stranded DNA. In some examples, the Cas includes Cas9.

Some examples herein provide a composition. The composition may include a set of at least about 1,000,000 WG fragments each having approximately the same number of base pairs as one another.

In some examples, the number of base pairs is between about 100 and about 200. In some examples, the number of base pairs is between about 200 and about 400. In some examples, the number of base pairs is between about 500 and about 700.

In some examples, the WG includes double-stranded DNA.

In some examples, the number of base pairs in the WG fragments of the set of WG fragments varies by less than about 20%. In some examples, the number of base pairs in the WG fragments of the set of WG fragments varies by less than about 10%. In some examples, the number of base pairs in the WG fragments of the set of WG fragments varies by less than about 5%.

Such a composition may be prepared according to methods such as described above.

Some examples herein provide a method of cutting molecules of a target polynucleotide having a sequence. The method may include contacting, in a fluid, first and second molecules of the target polynucleotide with a plurality of first and second CRISPR-associated protein guide RNA ribonucleoproteins (Cas-gRNA RNPs). The method may include hybridizing one of the first Cas-gRNA RNPs to a first subsequence in the first molecule. The method may include hybridizing one of the second Cas-gRNA RNPs to a second subsequence in the second molecule. The second subsequence may only partially overlap with the first subsequence. The method may include inhibiting, by the one of the first Cas-gRNA RNPs, hybridization of any of the second Cas-gRNA RNPs to the second subsequence in the first molecule. The method may include inhibiting, by the one of the second Cas-gRNA RNPs, hybridization of any of the first Cas-gRNA RNPs to the first subsequence in the second molecule. The method may include cutting the first molecule at the first subsequence. The method may include cutting the second molecule at the second subsequence.

In some examples, the cut in the first molecule is at a different location in the sequence of the target polynucleotide than the cut in the second molecule. In some examples, the cut in the first molecule is offset from the cut in the second molecule by between about two base pairs and about ten base pairs in the sequence of the target polynucleotide.

In some examples, the first molecule is cut using the one of the first Cas-gRNA RNPs, and the second molecule is cut using the one of the second Cas-gRNA RNPs.

In some examples, the target polynucleotide includes double-stranded DNA. In some examples, the Cas includes Cas9 or dCas9.

In some examples, the method further includes contacting, in the fluid, the first and second molecules of the target polynucleotide with a plurality of third and fourth Cas-gRNA RNPs. The method further may include hybridizing one of the third Cas-gRNA RNPs to a third subsequence in the first molecule. The method further may include inhibiting, by the one of the third Cas-gRNA RNPs, hybridization of any of the fourth Cas-gRNA RNPs to a fourth subsequence in the first molecule. The fourth subsequence may only partially overlap with the third subsequence. The method may include cutting the first molecule at the third subsequence using the one of the third Cas-gRNA RNPs to generate a first fragment.

In some examples, the method further includes contacting, in the fluid, the first and second molecules of the target polynucleotide with a plurality of third and fourth Cas-gRNA RNPs. The method may include hybridizing one of the fourth Cas-gRNA RNPs to a fourth subsequence in the first molecule. The method may include inhibiting, by the one of the fourth Cas-gRNA RNPs, hybridization of any of the third Cas-gRNA RNPs to a third subsequence in the first molecule. The method may include cutting the first molecule at the fourth subsequence using the one of the fourth Cas-gRNA RNPs to generate a first fragment.

In some examples, the method further includes hybridizing one of the third Cas-gRNA RNPs to the third subsequence in the second molecule. The method further may include inhibiting, by the one of the third Cas-gRNA RNPs, hybridization of any of the fourth Cas-gRNA RNPs to the fourth subsequence in the second molecule. The method further may include cutting the second molecule at the third subsequence using the one of the third Cas-gRNA RNPs to generate a second fragment.

In some examples, the method further includes hybridizing one of the fourth Cas-gRNA RNPs to the fourth subsequence in the second molecule. The method further may include inhibiting, by the one of the fourth Cas-gRNA RNPs, hybridization of any of the third Cas-gRNA RNPs to the third subsequence in the second molecule. The method further may include cutting the second molecule at the fourth subsequence using the one of the fourth Cas-gRNA RNPs to generate a second fragment.

In some examples, the method further includes, while the one of the first Cas-gRNA RNPs and the one of the third or the fourth Cas-gRNA RNPs are hybridized to the first molecule, degrading any portions of the first molecule that are not between the one of the first Cas-gRNA RNPs and the one of the third or the fourth Cas-gRNA RNPs.

In some examples, the method further includes while the one of the second Cas-gRNA RNPs and the one of the third or the fourth Cas-gRNA RNPs are hybridized to the second molecule, degrading any portions of the second molecule that are not between the one of the second Cas-gRNA RNPs and the one of the third or the fourth Cas-gRNA RNPs. In some examples, the degrading is performed using exonuclease III or exonuclease VII.

In some examples, the first molecule is cut using the one of the third or the fourth Cas-gRNA RNPs, and the second molecule is cut using the one of the third or the fourth Cas-gRNA RNPs.

In some examples, the first and second fragments include different numbers of base pairs than one another. In some examples, the first fragment has a length of between about 100 base pairs and about 1000 base pairs, and the second fragment has a length between about 100 base pairs and about 1000 base pairs. In some examples, the first fragment has a length of between about 500 base pairs and about 700 base pairs, and the second fragment has a length between about 500 base pairs and about 700 base pairs. In some examples, the first fragment has a length of between about 200 base pairs and about 400 base pairs, and the second fragment has a length between about 200 base pairs and about 400 base pairs. In some examples, the first fragment has a length of between about 100 base pairs and about 200 base pairs, and the second fragment has a length between about 100 base pairs and about 200 base pairs.

Some examples herein provide a method of sequencing a target polynucleotide. The method may include generating first and second fragments of the target polynucleotide using methods described above. The method further may include ligating amplification adapters to ends of the first and second fragments. The method further may include respectively generating amplicons of the first and second fragments having the amplification adapters ligated thereto. The method further may include sequencing the amplicons of the first and second fragments.

In some examples, the method further includes using the first, second, third, and fourth subsequences, identifying the amplicons of the first fragment as deriving from the first molecule and identifying the amplicons of the second fragment as deriving from the second molecule.

In some examples, the method further includes ligating unique molecular identifiers (UMIs) to the ends of the first and second fragments prior to generating the amplicons. The method further may include using the UMIs, identifying the amplicons of the first fragment as deriving from the first molecule and identifying the amplicons of the second fragment as deriving from the second molecule. In some examples, the UMIs are coupled to, and ligated to the ends of the first and second fragments in the same operation as, the amplification adapters.

Some examples herein provide a composition. The composition may include first and second molecules of a target polynucleotide having a sequence. The composition may include a plurality of first and second CRISPR-associated protein guide RNA ribonucleoproteins (Cas-gRNA RNPs). One of the first Cas-gRNA RNPs may be hybridized to a first subsequence in the first molecule and may inhibit hybridization of any of the second Cas-gRNA RNPs to a second subsequence in the first molecule. The second subsequence may only partially overlap with the first subsequence. One of the second Cas-gRNA RNPs may be hybridized to the second subsequence in the second molecule and may inhibit hybridization of any of the first Cas-gRNA RNPs to the first subsequence in the second molecule.

In some examples, the cut in the first molecule is at a different location in the sequence of the target polynucleotide than the cut in the second molecule. In some examples, the cut in the first molecule is offset from the cut in the second molecule by between about two base pairs and about ten base pairs in the sequence of the target polynucleotide.

In some examples, the one of the first Cas-gRNA RNPs is for cutting the first molecule, and the one of the second Cas-gRNA RNPs is for cutting the second molecule.

In some examples, the target polynucleotide includes double-stranded DNA. In some examples, the Cas includes Cas9 or dCas9.

In some examples, the composition further includes a plurality of third and fourth Cas-gRNA RNPs. One of the third Cas-gRNA RNPs may be hybridized to a third subsequence in the first molecule, may inhibit hybridization of any of the fourth Cas-gRNA RNPs to a fourth subsequence in the first molecule, and may be for cutting the first molecule at the third subsequence to generate a first fragment. The fourth subsequence may only partially overlap with the third subsequence.

In some examples, the composition further includes a plurality of third and fourth Cas-gRNA RNPs. One of the fourth Cas-gRNA RNPs may be hybridized to a fourth subsequence in the first molecule, may inhibit hybridization of any of the third Cas-gRNA RNPs to a third subsequence in the first molecule, and may be for cutting the first molecule at the fourth subsequence to generate a first fragment. The fourth subsequence may only partially overlap with the third subsequence.

In some examples, one of the third Cas-gRNA RNPs may be hybridized to the third subsequence in the second molecule, may inhibit hybridization of any of the fourth Cas-gRNA RNPs to the fourth subsequence in the second molecule, and may be for cutting the second molecule at the third subsequence to generate a second fragment.

In some examples, one of the fourth Cas-gRNA RNPs may be hybridized to the fourth subsequence in the second molecule, may inhibit hybridization of any of the third Cas-gRNA RNPs to the third subsequence in the second molecule, and may be for cutting the second molecule at the fourth subsequence to generate a second fragment.

In some examples, the composition further includes an exonuclease for degrading any portions of the first molecule that are not between the one of the first Cas-gRNA RNPs and the one of the third or the fourth Cas-gRNA RNPs.

In some examples, the composition further includes an exonuclease for degrading any portions of the second molecule that are not between the one of the second Cas-gRNA RNPs and the one of the third or the fourth Cas-gRNA RNPs.

In some examples, the exonuclease includes exonuclease III or exonuclease VII.

In some examples, the one of the third or the fourth Cas-gRNA RNPs is for cutting the first molecule, and the one of the third or the fourth Cas-gRNA RNPs is for cutting the second molecule.

In some examples, the first and second fragments include different numbers of base pairs than one another. In some examples, the first fragment has a length of between about 100 base pairs and about 1000 base pairs, and the second fragment has a length between about 100 base pairs and about 1000 base pairs. In some examples, the first fragment has a length of between about 500 base pairs and about 700 base pairs, and the second fragment has a length between about 500 base pairs and about 700 base pairs. In some examples, the first fragment has a length of between about 200 base pairs and about 400 base pairs, and the second fragment has a length between about 200 base pairs and about 400 base pairs. In some examples, the first fragment has a length of between about 100 base pairs and about 200 base pairs, and the second fragment has a length between about 100 base pairs and about 200 base pairs.

Some examples herein provide a composition. The composition may include first and second molecules of a target polynucleotide having a sequence. The first molecule may have a first end at a first subsequence. The second molecule may have a first end at a second subsequence. The first subsequence may only partially overlap with the second subsequence.

In some examples, the first end of the first molecule is at a different location in the sequence of the target polynucleotide than the first end of the second molecule. In some examples, the first end of the first molecule is offset from the first end of the second molecule by between about two base pairs and about ten base pairs in the sequence of the target polynucleotide.

In some examples, the first molecule further has a second end at a third subsequence. The second molecule further may have a second end at the third subsequence or at a fourth subsequence. The third subsequence may only partially overlap with the fourth subsequence. In some examples, the second end of the first molecule is at a different location in the sequence of the target polynucleotide than the second end of the second molecule. In some examples, the second end of the first molecule is offset from the second end of the second molecule by between about two base pairs and about ten base pairs in the sequence of the target polynucleotide.

In some examples, the target polynucleotide includes double-stranded DNA.

In some examples, the first and second molecules include different numbers of base pairs than one another. In some examples, the first molecule has a length of between about 100 base pairs and about 1000 base pairs, and the second molecule has a length between about 100 base pairs and about 1000 base pairs. In some examples, the first fragment has a length of between about 500 base pairs and about 700 base pairs, and the second fragment has a length between about 500 base pairs and about 700 base pairs. In some examples, the first fragment has a length of between about 200 base pairs and about 400 base pairs, and the second fragment has a length between about 200 base pairs and about 400 base pairs. In some examples, the first fragment has a length of between about 100 base pairs and about 200 base pairs, and the second fragment has a length between about 100 base pairs and about 200 base pairs.

Some examples herein provide a method of generating a fragment of a target polynucleotide having a sequence. The method may include contacting, in a fluid, the target polynucleotide with first and second fusion proteins. The first fusion protein may include a first CRISPR-associated protein guide RNA ribonucleoprotein (Cas-gRNA RNP) coupled to a first transposase having a first amplification adapter coupled thereto. The second fusion protein may include a second Cas-gRNA RNP coupled to a second transposase having a second amplification adapter coupled thereto. The method may include, while promoting activity of the first and second Cas-gRNA RNPs and inhibiting activity of the first and second transposases, hybridizing the first Cas-gRNA RNP to a first subsequence in the target polynucleotide, and hybridizing the second Cas-gRNA RNP to a second subsequence in the target polynucleotide. The method may include then, while promoting activity of the first and second transposases, using the first transposase to add the first amplification adapter to a first location in the target polynucleotide, and using the second transposase to add the second amplification adapter to a second location in the target polynucleotide.

In some examples, activity of the Cas-gRNA RNPs is promoted and the activity of the transposases is inhibited using a first condition of the fluid. In some examples, the first condition of the fluid includes presence of a sufficient amount of calcium ions, manganese ions, or both calcium and manganese ions for activity of the Cas-gRNA RNPs. In some examples, the first condition of the fluid includes absence of a sufficient amount of magnesium ions for activity of the transposases.

In some examples, activity of the transposases is promoted using a second condition of the fluid. In some examples, the second condition of the fluid includes presence of a sufficient amount of magnesium ions for activity of the transposases.

In some examples, the method further includes, while the Cas-gRNA RNP of the first fusion protein is hybridized to the first subsequence and the Cas-gRNA RNP of the second fusion protein is hybridized to the second subsequence, degrading any portions of the target polynucleotide that are not between the Cas-gRNA RNPs of the first and second fusion proteins. In some examples, the degrading is performed using exonuclease III or exonuclease VII.

In some examples, the method further includes releasing the target polynucleotide from the first and second fusion proteins to provide a fragment of the target polynucleotide having the first amplification adapter at one end, and the second amplification adapter at the other end. In some examples, the releasing is performed using proteinase K, sodium dodecyl sulfate (SDS), or both proteinase K and SDS.

In some examples, the fragment has a length of between about 100 base pairs and about 1000 base pairs. In some examples, the fragment has a length of between about 500 base pairs and about 700 base pairs. In some examples, the fragment has a length of between about 200 base pairs and about 400 base pairs. In some examples, the fragment has a length of between about 100 base pairs and about 200 base pairs.

In some examples, the Cas includes dCas9. In some examples, the transposase includes Tn5.

In some examples, the first amplification adapter includes a P5 adapter, and the second amplification adapter includes a P7 adapter.

In some examples, the first amplification adapter includes a first unique molecular identifier (UMI), and the second amplification adapter includes a second UMI.

In some examples, the first location is within about 10 bases of the first subsequence, and the second location is within about 10 bases of the second subsequence.

In some examples, in each of the first and second fusion proteins, the Cas-gRNA RNP is coupled to the transposase via a covalent linkage.

In some examples, in each of the first and second fusion proteins, the Cas-gRNA RNP is coupled to the transposase via a non-covalent linkage. In some examples, the Cas-gRNA RNP is covalently coupled to an antibody and the transposase is covalently coupled to an antigen to which the antibody is non-covalently coupled, or the Cas-gRNA RNP is covalently coupled to an antigen and the transposase is covalently coupled to an antibody to which the antigen is non-covalently coupled. In some examples, the Cas-gRNA is non-covalently coupled to the transposase via hybridization between the gRNA and the first or second amplification adapter. In some examples, the Cas-gRNA is non-covalently coupled to the transposase via hybridization between the gRNA and an oligonucleotide within the transposase.

In some examples, in the first fusion protein, a portion of the gRNA that hybridizes to the first subsequence has a length of about 15 to about 18 nucleotides, and in the second fusion protein, a portion of the gRNA that hybridizes to the second subsequence has a length of about 15 to about 18 nucleotides.

In some examples, the first and second fusion proteins are in an approximately stoichiometric ratio to the target polynucleotide.

In some examples, the target polynucleotide includes double-stranded DNA.

In some examples, a first tag is coupled to the first Cas-gRNA RNP and a second tag is coupled to the second Cas-gRNA RNP. In some examples, the method includes coupling the first tag to a first tag partner coupled to a substrate, and coupling the second tag to a second tag partner coupled to the substrate. In some examples, the coupling is performed after the first and second Cas-gRNA RNPs respectively are hybridized to the first and second subsequences. In some examples, the first and amplification adapters are added after the first and second tags respectively are added to the first and second tag partners.

In some examples, the first and second tags include biotin. In some examples, the first and second tag partners include streptavidin. In some examples, the substrate includes a bead. In some examples, the Cas-gRNA RNP includes Cas12k. In some examples, the transposase includes Tn5 or a Tn7 like transposase.

Some examples herein provide a method of sequencing a target polynucleotide. The method may include generating a fragment of the target polynucleotide using one of the foregoing methods, generating amplicons of the fragment, and sequencing the amplicons.

Some examples herein provide a composition. The composition may include a target polynucleotide having a sequence. The composition may include a first fusion protein including a first CRISPR-associated protein guide RNA ribonucleoprotein (Cas-gRNA RNP) coupled to a first transposase having a first amplification adapter coupled thereto. The first Cas-gRNA RNP may be hybridized to a first subsequence in the target polynucleotide.

In some examples, the composition may include a second fusion protein including a second Cas-gRNA RNP coupled to a second transposase having a second amplification adapter coupled thereto. The second Cas-gRNA RNP may be hybridized to a second subsequence in the target polynucleotide.

In some examples, the composition further includes a fluid having a condition promoting activity of the first Cas-gRNA RNP and inhibiting activity of the first transposase. In some examples, the condition of the fluid includes presence of a sufficient amount of calcium ions, manganese ions, or both calcium and manganese ions for activity of the first Cas-gRNA RNP. In some examples, the condition of the fluid includes absence of a sufficient amount of magnesium ions for activity of the first transposase.

In some examples, the composition further includes a fluid having a condition promoting activity of the first transposase, and in which the first transposase adds the first amplification adapter to a first location in the target polynucleotide. In some examples, the condition of the fluid includes presence of a sufficient amount of magnesium ions for activity of the first transposase.

In some examples, the composition further includes an agent for releasing the target polynucleotide from the first and second fusion proteins to provide a fragment of the target polynucleotide having the first amplification adapter at one end, and the second amplification adapter at the other end. In some examples, the agent includes proteinase K, sodium dodecyl sulfate (SDS), or both proteinase K and SDS.

In some examples, the fragment has a length of between about 100 base pairs and about 1000 base pairs. In some examples, the fragment has a length of between about 500 base pairs and about 700 base pairs. In some examples, the fragment has a length of between about 200 base pairs and about 400 base pairs. In some examples, the fragment has a length of between about 100 base pairs and about 200 base pairs.

In some examples, the composition further includes an exonuclease for degrading any portions of the target polynucleotide that are not between the first and second Cas-gRNA RNPs. In some examples, the exonuclease includes exonuclease III or exonuclease VII.

In some examples, the Cas includes dCas9. In some examples, the transposase includes Tn5.

In some examples, the first adapter includes a P5 adapter, and the second adapter includes a P7 adapter.

In some examples, the first amplification adapter includes a first unique molecular identifier (UMI), and the second amplification adapter includes a second UMI.

In some examples, the first location is within about 10 bases of the first subsequence, and the second location is within about 10 bases of the second subsequence.

In some examples, the first Cas-gRNA RNP is coupled to the first transposase via a covalent linkage.

In some examples, the first Cas-gRNA RNP is coupled to the first transposase via a non-covalent linkage. In some examples, the first Cas-gRNA RNP is covalently coupled to an antibody and the first transposase is covalently coupled to an antigen to which the antibody is non-covalently coupled, or the Cas-gRNA RNP is covalently coupled to an antigen and the first transposase is covalently coupled to an antibody to which the antigen is non-covalently coupled. In some examples, the first Cas-gRNA is non-covalently coupled to the transposase via hybridization between the gRNA and the first or second amplification adapter. In some examples, the first Cas-gRNA is non-covalently coupled to the transposase via hybridization between the gRNA and an oligonucleotide within the transposase.

In some examples, in the first fusion protein, a portion of the gRNA that hybridizes to the first subsequence has a length of about 15 to about 18 nucleotides. In examples including the second fusion protein, a portion of the gRNA that hybridizes to the second subsequence has a length of about 15 to about 18 nucleotides.

In some examples, the first fusion protein is in an approximately stoichiometric ratio to the target polynucleotide.

In some examples, the target polynucleotide includes double-stranded DNA.

Some examples further include a first tag coupled to the first Cas-gRNA RNP. Some examples further include a substrate and a first tag partner coupled to the substrate and to the first tag.

Some examples further include a second tag coupled to the second Cas-gRNA RNP. Some examples further include a substrate, a first tag partner coupled to the substrate and to the first tag, and a second tag partner coupled to the substrate and to the second tag.

In some examples, the first and second tags include biotin. In some examples, the first and second tag partners include streptavidin. In some examples, the substrate includes a bead. In some examples, the Cas-gRNA RNP includes Cas12k. In some examples, the transposase includes Tn5 or a Tn7 like transposase.

Some examples herein provide a method of characterizing proteins coupled to respective loci of a target polynucleotide. The method may include contacting the target polynucleotide with first and second CRISPR-associated protein guide RNA ribonucleoproteins (Cas-gRNA RNPs). The method may include respectively hybridizing the first and second Cas-gRNA RNPs to first and second subsequences in the target polynucleotide, the proteins may be coupled to respective loci of the target polynucleotide between the first and second subsequences. The method may include cutting the target polynucleotide at the first subsequence using the first Cas-gRNA RNP and at the second subsequence using the second Cas-gRNA RNP to form a fragment. The proteins may be coupled to respective loci of the fragment. The method may include using corresponding oligonucleotides to respectively label each of the proteins coupled to the respective loci of the fragment. The method may include sequencing the corresponding oligonucleotides.

In some examples, the method includes enriching the fragment before using the corresponding oligonucleotides to respectively label each of the proteins coupled to the respective loci of the fragment. In some examples, the first and second Cas-gRNA RNPs respectively are coupled to tags such that the fragment is coupled to the tags via the first and second Cas-gRNA RNPs. The enriching may include contacting the fragment, coupled to the tags via the first and second Cas-gRNA RNPs, with a substrate coupled to tag partners. The enriching may include coupling the tags to the tag partners to couple the fragment to the substrate. The enriching may include removing any portions of the target polynucleotide that are not coupled to the substrate.

In some examples, the method includes identifying the proteins using the corresponding oligonucleotides.

In some examples, the method includes identifying the loci using the corresponding oligonucleotides.

In some examples, the method includes quantifying the proteins using the corresponding oligonucleotides.

In some examples, using corresponding oligonucleotides to respectively label each of the proteins includes contacting the fragment with a mixture of antibodies that are specific to different proteins. Each of the antibodies may be coupled to a corresponding oligonucleotide. For any antibodies in the mixture that are specific to the proteins coupled to the respective loci of the fragment, those antibodies and the corresponding oligonucleotides may be coupled to those proteins. In some examples, a plurality of the proteins are coupled to a respective one of the loci, and a plurality of antibodies in the mixture are coupled to the proteins at that locus.

In some examples, sequencing the corresponding oligonucleotides includes hybridizing the corresponding oligonucleotides to a bead array. In some examples, sequencing the corresponding oligonucleotides includes performing sequencing-by-synthesis on the corresponding oligonucleotides.

In some examples, the corresponding oligonucleotides include unique molecular identifiers (UMIs).

In some examples, the method includes using respective presences of the corresponding oligonucleotides to identify the proteins.

In some examples, the method includes using respective quantities of the corresponding oligonucleotides to quantify the proteins.

In some examples, using corresponding oligonucleotides to respectively label each of the proteins includes: contacting the fragment with a plurality of transposases, each of the transposases being coupled to a corresponding oligonucleotide; inhibiting, by the proteins coupled to the respective loci of the fragment, activity of the transposases at the loci; and at locations other than the loci, using the transposases to add the corresponding oligonucleotides to the fragment.

In some examples, sequencing the corresponding oligonucleotides includes performing sequencing-by-synthesis on the fragment to which the corresponding oligonucleotides are added.

In some examples, using respective locations in the fragment of the corresponding oligonucleotides to identify the respective loci of the proteins.

In some examples, the transposases divide the fragment into subfragments and the sequencing-by-synthesis is performed on the subfragments.

In some examples, the corresponding oligonucleotides include amplification adapters. In some examples, the amplification adapters include P5 and P7 adapters.

In some examples, the amplification adapters include unique molecular identifiers (UMIs).

In some examples, the Cas includes Cas9.

In some examples, the fragment has a length of between about 100 base pairs and about 1000 base pairs. In some examples, the fragment has a length of between about 500 base pairs and about 700 base pairs. In some examples, the fragment has a length of between about 200 base pairs and about 400 base pairs. In some examples, the fragment has a length of between about 100 base pairs and about 200 base pairs.

In some examples, the target polynucleotide includes double-stranded DNA.

Some examples herein provide a composition. The composition may include a fragment of a target polynucleotide. Proteins may be coupled to respective loci of the fragment. The composition may include a mixture of antibodies that are specific to different proteins. Each of the antibodies may be coupled to a corresponding oligonucleotide. For any antibodies in the mixture that are specific to the proteins coupled to the respective loci of the fragment, those antibodies and the corresponding oligonucleotides are coupled to those proteins.

In some examples, a plurality of the proteins are coupled to a respective one of the loci, and a plurality of antibodies in the mixture are coupled to the proteins at that locus.

In some examples, the corresponding oligonucleotides include unique molecular identifiers (UMIs).

In some examples, respective presences of the corresponding oligonucleotides are usable to identify the proteins.

In some examples, respective quantities of the corresponding oligonucleotides are usable to quantify the proteins.

In some examples, the fragment has a length of between about 100 base pairs and about 1000 base pairs. In some examples, the fragment has a length of between about 500 base pairs and about 700 base pairs. In some examples, the fragment has a length of between about 200 base pairs and about 400 base pairs. In some examples, the fragment has a length of between about 100 base pairs and about 200 base pairs.

In some examples, the target polynucleotide includes double-stranded DNA.

Some examples herein provide a composition. The composition may include a fragment of a target polynucleotide. Proteins may be coupled to respective loci of the fragment. The composition may include plurality of transposases. Each of the transposases may be coupled to a corresponding oligonucleotide. The proteins coupled to the respective loci of the fragment may inhibit activity of the transposases at the loci. The transposases may add the corresponding oligonucleotides to the fragment at locations other than the loci.

In some examples, respective locations in the fragment of the corresponding oligonucleotides are usable to identify the respective loci of the proteins.

In some examples, the transposases divide the fragment into subfragments.

In some examples, the corresponding oligonucleotides include amplification adapters. In some examples, the amplification adapters include P5 and P7 adapters. In some examples, the amplification adapters include unique molecular identifiers (UMIs).

In some examples, the transposases include Tn5.

In some examples, the fragment has a length of between about 100 base pairs and about 1000 base pairs. In some examples, the fragment has a length of between about 500 base pairs and about 700 base pairs. In some examples, the fragment has a length of between about 200 base pairs and about 400 base pairs. In some examples, the fragment has a length of between about 100 base pairs and about 200 base pairs.

In some examples, the target polynucleotide includes double-stranded DNA.

Some examples herein provide a composition that includes a target polynucleotide having a plurality of subsequences. The composition may include a plurality of complexes each including an ShCAST (Scytonema hofmanni CRISPR associated transposase) coupled to guide RNA (gRNA). The ShCAST may have an amplification adapter coupled thereto. Each of the complexes may be hybridized to a corresponding one of the subsequences in the target polynucleotide.

In some examples, the composition further includes a fluid having a condition promoting hybridization of the complexes to the subsequences and inhibiting activity of the transposases. In some examples, the condition of the fluid includes absence of a sufficient amount of magnesium ions for activity of the transposases.

In some examples, the composition further includes a fluid having a condition promoting activity of the transposases, and in which the transposases add the amplification adapters to locations in the target polynucleotide. In some examples, the condition of the fluid includes presence of a sufficient amount of magnesium ions for activity of the transposases.

In some examples, the ShCAST includes Cas12k. In some examples, the transposase includes Tn5 or a Tn7 like transposase. In some examples, the adapter includes at least one of a P5 adapter and a P7 adapter. In some examples, the target polynucleotide includes double-stranded DNA.

In some examples, at least one of the gRNA and the transposase is biotinylated. The composition further may include a streptavidin-coated bead to which the at least one of the gRNA and transposase that is biotinylated is coupled.

Some examples herein provide a method of generating a fragment of a double-stranded polynucleotide. The method may include coupling the double-stranded polynucleotide to a substrate. The method may include respectively hybridizing first and second CRISPR-associated protein guide RNA ribonucleoprotein (Cas-gRNA RNP) nickases to first and second subsequences in the double-stranded polynucleotide. The first subsequence may be 3′ of a target sequence along a first strand of the double-stranded polynucleotide. The second subsequence may be 3′ of the target sequence along a second strand of the double-stranded polynucleotide. The method may include cutting the first strand at the first subsequence using the first Cas-gRNA RNP nickase. The method may include cutting the second strand at the second subsequence using the second Cas-gRNA RNP nickase. The method may include using a polymerase to extend the first and second strands from the respective cuts and elute the target sequence from the substrate. The method may include sequencing the eluted target sequence.

In some examples, the substrate includes a bead, for example a paramagnetic bead.

In some examples, 3′ ends of the double-stranded polynucleotide are coupled to tags and the substrate is coupled to tag partners, the coupling including coupling the tags to the tag partners. In some examples, the tags include biotin, and the tag partners include streptavidin.

In some examples, the first and second Cas-gRNA RNP nickases include Cas9.

In some examples, the polymerase includes a strand displacement polymerase. In some examples, the polymerase includes Vent or Bsu.

In some examples, the polymerase has 5′ exonuclease activity. In some examples, the polymerase includes Taq, Bst, or DNA Polymerase I.

Some examples provide a composition. The composition may include a double-stranded polynucleotide coupled to a substrate. The composition may include first and second CRISPR-associated protein guide RNA ribonucleoprotein (Cas-gRNA RNP) nickases respectively hybridized to first and second subsequences in the double-stranded polynucleotide. The first subsequence may be 3′ of a target sequence along a first strand of the double-stranded polynucleotide. The second subsequence may be 3′ of the target sequence along a second strand of the double-stranded polynucleotide.

In some examples, the substrate includes a bead, for example a paramagnetic bead.

In some examples, 3′ ends of the double-stranded polynucleotide are coupled to tags and the substrate is coupled to tag partners that are coupled to the tags. In some examples, the tags include biotin, and the tag partners include streptavidin.

In some examples, the first and second Cas-gRNA RNP nickases include Cas9.

Some examples provide a method of generating a fragment of a double-stranded polynucleotide. The method may include respectively hybridizing first and second complexes to first and second subsequences in the double-stranded polynucleotide. Each of the first and second complexes may include a CRISPR-associated protein guide RNA ribonucleoprotein (Cas-gRNA RNP) coupled to an amplification adaptor. The method may include respectively ligating the amplification adaptors of the hybridized first and second complexes to first and second ends of the double-stranded polynucleotide. The method may include removing the Cas-gRNA RNPs of the first and second complexes from the double-stranded polynucleotide. The method may include sequencing the double-stranded polynucleotide having the amplification adaptors ligated thereto.

In some examples, the first subsequence is 3′ of a target sequence along a first strand of the double-stranded polynucleotide, and the second subsequence is 3′ of the target sequence along a second strand of the double-stranded polynucleotide.

In some examples, the amplification adaptors are Y-shaped.

In some examples, each complex further includes a linker coupling the Cas-gRNA RNP to the amplification adapter. In some examples, the linker is coupled to the Cas of the Cas-gRNA RNP. In some examples, the linker is coupled to the gRNA. In some examples, the linker includes a protein, a polynucleotide, or a polymer. In some examples, the linker remains coupled to the amplification adaptor when the Cas-gRNA RNP is removed.

In some examples, the ligating includes using a ligase. In some examples, the ligase is present during the hybridizing. In some examples, the ligase is inactive during the hybridizing and is activated for the ligating using ATP. In some examples, the ligase is added after the hybridizing.

In some examples, the method includes A-tailing the double-stranded polynucleotide prior to the hybridizing, and wherein the amplification adaptor includes an unpaired T to hybridize with the A-tail. Alternatively, the amplification adaptor may be ligated to a blunt end.

In some examples, the amplification adaptor includes a unique molecular identifier. For example, the amplification adaptor may include a duplex unique molecular identifier.

In some examples, the Cas-gRNA RNP includes dCas9.

Some examples provide a composition. The composition may include a fragment of a double-stranded polynucleotide. The composition may include first and second complexes hybridized to first and second subsequences in the double-stranded polynucleotide. Each of the first and second complexes may include a CRISPR-associated protein guide RNA ribonucleoprotein (Cas-gRNA RNP) coupled to an amplification adaptor.

In some examples, the first subsequence is 3′ of a target sequence along a first strand of the double-stranded polynucleotide, and the second subsequence is 3′ of the target sequence along a second strand of the double-stranded polynucleotide.

In some examples, the amplification adaptors are Y-shaped.

In some examples, each complex further includes a linker coupling the Cas-gRNA RNP to the amplification adapter. In some examples, the linker is coupled to the Cas of the Cas-gRNA RNP. In some examples, the linker is coupled to the gRNA. In some examples, the linker includes a protein, a polynucleotide, or a polymer.

In some examples, the double-stranded polynucleotide includes an A-tail, and wherein the amplification adaptor includes an unpaired T to hybridize with the A-tail. Alternatively, the amplification adaptor may be ligated to a blunt end.

In some examples, the amplification adaptor includes a unique molecular identifier. For example, the amplification adaptor may include a duplex unique molecular identifier.

In some examples, the Cas-gRNA RNP includes dCas9.

Some examples herein provide a method of generating a fragment of a polynucleotide. The method may include hybridizing a first CRISPR-associated protein guide RNA ribonucleoprotein (Cas-gRNA RNP) to a first sequence in the polynucleotide. The method may include hybridizing a second Cas-gRNA RNP to a second sequence in the polynucleotide that is spaced apart from the first sequence by at least a target sequence. The method may include cutting the first and second sequences with the first and second Cas-gRNA RNPs to generate a fragment including first and second ends and the target sequence therebetween. The first end may have a first 5′ overhang of at least one base. The second end may have a second 5′ overhang of at least one base.

In some examples, the first and second 5′ overhangs are each about 2-5 bases in length. In some examples, the first and second 5′ overhangs are each about 5 bases in length.

In some examples, the first and second 5′ overhangs have different sequences than one another.

Some examples further include ligating a first amplification adapter to the first end of the fragment and ligating a second amplification adapter to the second end of the fragment. The first amplification adapter may have a third 5′ overhang that is complementary to the first 5′ overhang. The second amplification adapter may have a fourth 5′ overhang that is complementary to the second 5′ overhang. The third and fourth 5′ overhangs may have different sequences than one another. Some examples further include generating amplicons of the fragment having the first and second amplification adapters ligated thereto; sequencing the amplicons; and identifying the target polynucleotide based on the sequencing. In some examples, the amplification adapters include unique molecular identifiers (UMIs).

In some examples, the Cas includes Cas12a.

Some examples herein provide a composition. The composition may include a polynucleotide. The composition may include a first CRISPR-associated protein guide RNA ribonucleoprotein (Cas-gRNA RNP) hybridized to a first sequence in the polynucleotide. The composition may include a second Cas-gRNA RNP hybridized to a second sequence in the polynucleotide that is spaced apart from the first sequence by at least a target sequence. The first and second Cas-gRNA RNPs respectively may be being for cutting the first and second sequences of the polynucleotide to generate a fragment having first and second ends with the target sequence therebetween. The first end may have a first 5′ overhang of at least one base. The second end may have a second 5′ overhang of at least one base.

In some examples, the first and second 5′ overhangs are each about 2-5 bases in length. In some examples, the first and second 5′ overhangs are each about 5 bases in length.

In some examples, the first and second 5′ overhangs have different sequences than one another.

In some examples, the Cas includes Cas12a.

Some examples herein provide a composition. The composition may include a polynucleotide fragment each having first and second ends with the target sequence therebetween. The first end may have a first 5′ overhang of at least one base. The second end may have a second 5′ overhang of at least one base. The first and second 5′ overhangs may have different sequences than one another. The composition also may include a first amplification adaptor having a third 5′ overhang that is complementary to the first 5′ overhang and is not complementary to the second 5′ overhang. The composition also may include a second amplification adaptor having a fourth 5′ overhang that is complementary to the second 5′ overhang and is not complementary to the first 5′ overhang.

Some examples further include at least one ligase for ligating the first amplification adaptor to the first end and for ligating the second amplification adaptor to the second end.

In some examples, the first and second 5′ overhangs are each about 2-5 bases in length. In some examples, the first and second 5′ overhangs are each about 5 bases in length.

In some examples, the first and second amplification adapters include unique molecular identifiers (UMIs).

In some examples, the ligase includes T4 DNA ligase.

Some examples herein provide a plurality of polynucleotide fragments each having first and second ends with the target sequence therebetween. The first end may have a first 5′ overhang of at least one base. The second end may have a second 5′ overhang of at least one base. The first and second 5′ overhangs may have different sequences than one another and than the first and second 5′ overhangs of other fragments.

Some examples further include a plurality of first amplification adaptors. Each of the first amplification adaptors may have a third 5′ overhang that is complementary to the first 5′ overhang of a corresponding fragment and is not complementary to the second 5′ overhang of that fragment and is not complementary to the first or second 5′ overhangs of other fragments. Some examples herein further include a plurality of second amplification adaptors. Each of the second amplification adaptors may have a fourth 5′ overhang that is complementary to the second 5′ overhang of a corresponding fragment and is not complementary to the first 5′ overhang of that fragment and is not complementary to the first or second 5′ overhangs of other fragments.

Some examples further include ligases for ligating the first amplification adaptors to the first ends for which the first and third 5′ overhangs are complementary and for ligating the second amplification adaptors to the second ends for which the second and fourth 5′ overhangs are complementary. In some examples, the ligase includes T4 DNA ligase.

In some examples, the first and second amplification adapters include unique molecular identifiers (UMIs). In some examples, the first and second 5′ overhangs are each about 2-5 bases in length. In some examples, the first and second 5′ overhangs are each about 5 bases in length.

Some examples herein provide a composition. The composition may include a plurality of polynucleotides. The composition may include a plurality of first CRISPR-associated protein guide RNA ribonucleoprotein (Cas-gRNA RNPs) hybridized to respective first sequences in the polynucleotide. The composition may include a plurality of second Cas-gRNA RNPs hybridized to respective second sequences in the polynucleotide that are spaced apart from the respective first sequence by at least a respective target sequence. The first and second pluralities of Cas-gRNA RNPs respectively may be for cutting the first and second sequences of the respective polynucleotides to generate fragments respectively having first and second ends within the respective target sequence therebetween. The first end may have a first 5′ overhang of at least one base. The second end may have a second 5′ overhang of at least one base.

In some examples, the first and second 5′ overhangs are each about 2-5 bases in length. In some examples, the first and second 5′ overhangs are each about 5 bases in length.

In some examples, the first and second 5′ overhangs have different sequences than one another.

In some examples, the Cas includes Cas12a.

Some examples herein provide a guide RNA. The guide RNA may include a primer binding site, an amplification adaptor site, and a CRISPR protospacer.

In some examples, the primer binding site is approximately complementary to at least a portion of the CRISPR protospacer.

In some examples, the amplification adaptor site is located between the primer binding site and the CRISPR protospacer.

In some examples, the guide RNA includes at least one loop. In some examples, a first loop is located between the amplification adaptor site and the CRISPR protospacer. In some examples, a second loop is located between the amplification adaptor site and the CRISPR protospacer.

Some examples herein provide a CRISPR-associated protein guide RNA ribonucleoprotein (Cas-gRNA RNP). The Cas-gRNA RNP may include any one of the foregoing gRNAs, and a Cas protein binding the CRISPR protospacer.

In some examples, the Cas protein is configured to perform double-stranded polynucleotide cleavage. In some examples, the Cas protein includes Cas9, Cas 12a, or Cas12f.

In some examples, the primer binding site and the amplification adaptor site extend outside of the Cas protein.

Some examples herein provide a complex. The complex may include a polynucleotide including first and second strands. The complex may include a first CRISPR-associated protein guide RNA ribonucleoprotein (Cas-gRNA RNP). The first Cas-gRNA RNP may include a first guide RNA including a first primer binding site, a first amplification adaptor site, and a first CRISPR protospacer; and a first Cas protein binding the first CRISPR protospacer. The first CRISPR protospacer may be hybridized to the first strand and the first primer binding site may be hybridized to the second strand.

In some examples, the first and second strands are cut by the first Cas-gRNA RNP at respective locations based upon the sequence of the first CRISPR protospacer. In some examples, the first Cas protein includes Cas9, Cas 12a, or Cas12f.

In some examples, the complex further includes a first reverse transcriptase for creating an amplicon of the amplification adaptor site at the cut in the second strand caused by the first Cas protein. In some examples, the first reverse transcriptase is coupled to the first Cas protein. In some examples, the first reverse transcriptase and the first Cas protein are components of a first fusion protein.

In some examples, the first primer binding site is approximately complementary to at least a portion of the first CRISPR protospacer.

In some examples, the first amplification adaptor site is located between the first primer binding site and the first CRISPR protospacer.

In some examples, the first gRNA further includes at least one loop. In some examples, a first loop is located between the first amplification adaptor site and the first CRISPR protospacer. In some examples, a second loop is located between the first amplification adaptor site and the first CRISPR protospacer.

Some examples further include a second Cas-gRNA RNP. The second Cas-gRNA RNP may include a second guide RNA including a second primer binding site, a second amplification adaptor site, and a second CRISPR protospacer. The second Cas-gRNA RNP may include a second Cas protein binding the second CRISPR protospacer. The second CRISPR protospacer may be hybridized to the first strand and the second primer binding site may be hybridized to the second strand.

In some examples, the first and second strands are cut by the second Cas-gRNA RNP at respective locations based upon the sequence of the second CRISPR protospacer. In some examples, the cuts in the first and second strands by the second Cas-gRNA RNP are spaced apart from the cuts in the first and second strands by the first Cas-gRNA RNP by at least a target sequence. In some examples, the second Cas protein includes Cas9, Cas12a, or Cas12f.

In some examples, the complex further includes a second reverse transcriptase for creating an amplicon of the amplification adaptor site at the cut in the second strand caused by the second Cas protein. In some examples, the second reverse transcriptase is coupled to the second Cas protein. In some examples, the second reverse transcriptase and the second Cas protein are components of a second fusion protein.

In some examples, the second primer binding site is approximately complementary to at least a portion of the second CRISPR protospacer.

In some examples, the second amplification adaptor site is located between the second primer binding site and the second CRISPR protospacer.

Some examples herein provide a partially double-stranded polynucleotide fragment.

The fragment may include a first end including a first 3′ overhang; a second end; and a target sequence located between the first and second ends.

In some examples, the first 3′ overhang includes a first amplification adaptor.

In some examples, the second end includes a second 3′ overhang.

In some examples, the second 3′ overhang includes a second amplification adaptor.

Some examples herein provide a method. The method may include contacting a first CRISPR-associated protein guide RNA ribonucleoprotein (Cas-gRNA RNP) with a polynucleotide including first and second strands. The first Cas-gRNA may include a first guide RNA including a first primer binding site, a first amplification adaptor site, and a first CRISPR protospacer; and a first Cas protein binding the first CRISPR protospacer. The method may include hybridizing the first CRISPR protospacer to the first strand. The method may include hybridizing the first primer binding site to the second strand.

In some examples, the method further includes cutting the first and second strands, by the first Cas-gRNA RNP, at respective locations based upon the sequence of the first CRISPR protospacer. In some examples, the first Cas protein includes Cas9, Cas12a, or Cas12f.

In some examples, the method further includes using a first reverse transcriptase to generate an amplicon of the amplification adaptor site at the cut in the second strand caused by the first Cas protein. In some examples, the first reverse transcriptase is coupled to the first Cas protein. In some examples, the first reverse transcriptase and the first Cas protein are components of a first fusion protein.

In some examples, the first primer binding site is approximately complementary to at least a portion of the first CRISPR protospacer.

In some examples, the first amplification adaptor site is located between the first primer binding site and the first CRISPR protospacer.

In some examples, the first gRNA further includes at least one loop. In some examples, a first loop is located between the first amplification adaptor site and the first CRISPR protospacer. In some examples, a second loop is located between the first amplification adaptor site and the first CRISPR protospacer.

In some examples, the method further includes contacting the polynucleotide with a second Cas-gRNA RNP. The second Cas-gRNA RNP may include a second guide RNA including a second primer binding site, a second amplification adaptor site, and a second CRISPR protospacer; and a second Cas protein binding the second CRISPR protospacer. The method may include hybridizing the second CRISPR protospacer to the first strand. The method may include hybridizing the second primer binding site to the second strand.

In some examples, the method may include cutting the first and second strands, by the second Cas-gRNA RNP, at respective locations based upon the sequence of the second CRISPR protospacer. In some examples, the cuts in the first and second strands by the second Cas-gRNA RNP are spaced apart from the cuts in the first and second strands by the first Cas-gRNA RNP by at least a target sequence. In some examples, the second Cas protein includes Cas9, Cas12a, or Cas12f.

In some examples, the method further may include using a second reverse transcriptase to generate an amplicon of the amplification adaptor site at the cut in the second strand caused by the second Cas protein. In some examples, the second reverse transcriptase is coupled to the second Cas protein. In some examples, the second reverse transcriptase and the second Cas protein are components of a second fusion protein.

In some examples, the second primer binding site is approximately complementary to at least a portion of the second CRISPR protospacer.

In some examples, the second amplification adaptor site is located between the second primer binding site and the second CRISPR protospacer.

In some examples, the first and second Cas-gRNA RNPs and the first and second reverse transcriptases generate a partially double-stranded polynucleotide fragment having a first end and a second end. The first end may include a first 3′ overhang. The second end may include a second 3′ overhang. A target sequence may be located between the first and second ends. In some examples, the first 3′ overhang includes the amplicon of the first amplification adaptor site. In some examples, the second 3′ overhang includes the amplicon of the second amplification adaptor site. In some examples, the method further includes ligating a third amplification adaptor to a 5′ group at the first end; ligating a fourth amplification adaptor to a 5′ group at the second end; amplifying the fragment using the first, second, third, and fourth amplification adaptors; and sequencing the amplified fragment.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIGS. 1A-1K schematically illustrate example compositions and operations in a process flow for Cas-gRNA RNP mediated dehosting.

FIGS. 2A-2K schematically illustrate example compositions and operations in a process flow for WG fragmentation into different, defined fragment sizes.

FIGS. 3A-3E schematically illustrate example compositions and operations in a process flow for labeling polynucleotides using cuts.

FIGS. 4A-4J schematically illustrate example compositions and operations in a process flow for coupling amplification adapters to polynucleotides.

FIGS. 5A-5K schematically illustrate example compositions and operations in a process flow for targeted epigenetic assays.

FIGS. 6A-6B schematically illustrate example compositions and operations in a process flow for ShCAST (Scytonema hofmanni CRISPR associated transposase) targeted library preparation and enrichment.

FIGS. 7A-7H schematically illustrate example compositions and operations in another process flow for coupling amplification adapters to polynucleotides.

FIGS. 8A-8H schematically illustrate example compositions and operations in a process flow for enriching selected polynucleotide fragments using Cas-gRNA RNP nickases.

FIG. 9A schematically illustrates example compositions and operations in a previously known process flow for ligating amplification adaptors to fragments of a dsDNA library.

FIGS. 9B-9F schematically illustrate example compositions and operations in a process flow for ligating amplification adaptors to selected polynucleotide fragments using Cas-gRNA RNPs.

FIGS. 10A-10C schematically illustrate example compositions and operations in a process flow for generating fragments using Cas-gRNA RNPs and coupling adaptors thereto.

FIGS. 11A-11G schematically depict additional compositions and operations in a process flow for generating fragments using Cas-gRNA RNPs and coupling adaptors thereto.

FIG. 12 schematically depicts a target DNA fragment after tagmentation, stop and a TWB wash.

FIG. 13 schematically depicts a target DNA fragment after gap-fill and ligation with ELM.

FIG. 14 schematically depicts a Cas9 nickase (D10A) that cuts opposite the PAM sites.

FIG. 15 schematically depicts target DNA containing 3′ nicks.

FIG. 16 schematically depicts polymerase extension of the '3-ends that will result in elution of target fragments.

FIG. 17 depicts an example of an IGV trace showing enrichment of the four lambda targets.

### DETAILED DESCRIPTION

Genomic library preparation, and targeted epigenetic assays, using Cas-gRNA ribonucleoproteins (RNPs) are provided herein.

Regarding genomic library preparation, some examples herein relate to Cas-gRNA RNP mediated dehosting; some examples herein relate to fragmentation of a whole genome (WG) into different, defined fragment sizes; some examples herein relate to cutting polynucleotides; and some examples herein relate to coupling amplification adapters to polynucleotides. It will be appreciated that one or more aspects of any such examples relating to genomic library preparation may be used in combination with one or more aspects of any other such examples relating to genomic library preparation.

Regarding targeted epigenetic assays, some examples herein relate to using Cas-gRNA RNPs to enrich DNA regions (small or large) retaining epigenetic features (e.g., chromatin), which are subsequently processed in an epigenetic-NGS assay. This approach enables ultra-deep epigenetic assays, improving resolution of fine epigenetic changes (e.g., as compared to ATAC-seq or ChIP-seq) and complex networks (e.g., locus-associated proteomics) which may facilitate a better understanding of epigenetic mechanisms such as may be important research or clinical development. It will be appreciated that one or more aspects of any such examples relating to targeted epigenetic assays may be used in combination with one or more aspects of any examples relating to genomic library preparation, and vice versa.

First, some terms used herein will be briefly explained. Then, some example compositions and example methods for genomic library preparation, and targeted epigenetic assays, using Cas-RNPs will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to +10%, such as less than or equal to ±5%, such as less than or equal to ±2%, such as less than or equal to +1%, such as less than or equal to ±0.5%, such as less than or equal to ±0.2%, such as less than or equal to +0.1%, such as less than or equal to 0.05%.

As used herein, terms such as “hybridize” and “hybridization” are intended to mean noncovalently associating a polynucleotides to one another along the lengths of those polynucleotides to form a double-stranded “duplex,” a three-stranded “triplex,” or higher-order structure For example, two DNA polynucleotide strands may associate through complementary base pairing to form a duplex. The primary interaction between polynucleotide strands typically is nucleotide base specific, e.g., A:T, A:U, and G:C, by Watson-Crick and Hoogsteen-type hydrogen bonding. Base-stacking and hydrophobic interactions also may contribute to duplex stability. Hybridization conditions may include salt concentrations of less than about 1 M, more usually less than about 500 mM, or less than about 200 mM. A hybridization buffer may include a buffered salt solution such as 5% SSPE or other suitable buffer known in the art. Hybridization temperatures may be as low as 5° C., but are typically greater than 22° C., and more typically greater than about 30° C., and typically in excess of 37° C. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, and ThermoPhi DNA polymerase. In specific, nonlimiting examples, the polymerase is selected from a group consisting of Bst, Bsu, and Phi29. As the polymerase extends the hybridized strand, it can be beneficial to include single-stranded binding protein (SSB). SSB may stabilize the displaced (non-template) strand.

Example polymerases having strand displacing activity include, without limitation, Vent polymerase, Bsu polymerase, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Example polymerases having 5′ exonuclease activity include Taq, Bst, and DNA polymerase I. Some polymerases have an activity that degrades the strand behind them (3′ exonuclease activity). Some useful polymerases have been modified, either by mutation or otherwise, to reduce or eliminate 3′ and/or 5′ exonuclease activity. Polymerases may include reverse transcriptases (RTs). Nonlimiting examples of RTs include MMLV and mutants thereof, e.g., such as described in Anzalone et al., “Search-and-replace genome editing without double-strand breaks or donor DNA,” Nature 576: 149-157 (2019), the entire contents of which are incorporated by reference herein.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer. A “capture primer” is intended to mean a primer that is coupled to the substrate and may hybridize to a first adapter of the target polynucleotide, while an “orthogonal capture primer” is intended to mean a primer that is coupled to the substrate and may hybridize to a second adapter of that target polynucleotide. A first adapter may have a sequence that is complementary to that of the capture primer, and a second adapter may have a sequence that is complementary to that of the orthogonal capture primer. A capture primer and an orthogonal capture primer may have different and independent sequences than one another. Additionally, a capture primer and an orthogonal capture primer may differ from one another in at least one other property. For example, the capture primer and the orthogonal capture primer may have different lengths than one another; either the capture primer or the orthogonal capture primer may include a non-nucleic acid moiety (such as a blocking group or excision moiety) that the other of the capture primer or the orthogonal capture primer lacks; or any suitable combination of such properties. A modified capture primer additionally may include a plurality of naturally occurring nucleic acids such as, but not limited to, DNA.

In some examples, capture primers are P5 or P7 primers that are commercially available from Illumina, Inc. P5 and P7 primers are nonlimiting examples of primers that are orthogonal to one another. The P5 and P7 primer sequences may have the following sequences, in some examples:

### Paired Read Set:

P5:
(SEQ ID NO: 1)
5′-AATGATACGGCGACCACCGAGAUCTACAC-3′
P7:
(SEQ ID NO: 2)
5′-CAAGCAGAAGACGGCATACGAG*AT-3′

### Single Read Set:

P5:
(SEQ ID NO: 3)
5′-AATGATACGGCGACCACCGA-3′
P7:
(SEQ ID NO: 4)
5′-CAAGCAGAAGACGGCATACGA3′

where G* is G or 8-oxoguanine.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.” As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to capture, amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

The terms “sequence” and “subsequence” may in some cases be used interchangeably herein. For example, a sequence may include one or more subsequences therein. Each of such subsequences also may be referred to as a sequence.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, the term “protective element,” when used in reference to the 5′ or 3′ end of a polynucleotide, is intended to mean an element that inhibits modification of that end of the polynucleotide. Illustratively, the protective element may inhibit action of one or more enzymes upon that end of the polynucleotide, such as action of a 5′ or 3′ exonuclease. Non-limiting examples of protective elements include a hairpin sequence that is ligated to the 5′ and 3′ strands of the end of a double-stranded polynucleotide, a modified base (e.g., including a phosphorothioate bond or 3′ phosphate), or a dephosphorylated base.

As used herein, terms such as “CRISPR-Cas system,” “Cas-gRNA ribonucleoprotein,” and Cas-gRNA RNP refer to an enzyme system including a guide RNA (gRNA) sequence that includes an oligonucleotide sequence that is complementary or substantially complementary to a sequence within a target polynucleotide, and a Cas protein. CRISPR-Cas systems may generally be categorized into three major types which are further subdivided into ten subtypes, based on core element content and sequences; see, e.g., Makarova et al., “Evolution and classification of the CRISPR-Cas systems,” Nat Rev Microbiol. 9(6): 467-477 (2011). Cas proteins may have various activities, e.g., nuclease activity. Thus, CRISPR-Cas systems provide mechanisms for targeting a specific sequence (e.g., via the gRNA) as well as certain enzyme activities upon the sequence (e.g., via the Cas protein).

A Type I CRISPR-Cas system may include Cas3 protein with separate helicase and DNase activities. For example, in the Type 1-E system, crRNAs are incorporated into a multisubunit effector complex called Cascade (CRISPR-associated complex for antiviral defense), which binds to the target DNA and triggers degradation by the Cas3 protein; see, e.g., Brouns et al., “Small CRISPR RNAs guide antiviral defense in prokaryotes,” Science 321(5891): 960-964 (2008); Sinkunas et al., “Cas3 is a single-stranded DNA nuclease and ATP-dependent helicase in the CRISPR-Cas immune system,” EMBO J 30:1335-1342 (2011); and Beloglazova et al., “Structure and activity of the Cas3 HD nuclease MJ0384, an effector enzyme of the CRISPR interference, EMBO J 30:4616-4627 (2011). Type II CRISPR-Cas systems include the signature Cas9 protein, a single protein (about 160 KDa) capable of generating crRNA and cleaving the target DNA. The Cas9 protein typically includes two nuclease domains, a RuvC-like nuclease domain near the amino terminus and the HNH (or McrA-like) nuclease domain near the middle of the protein. Each nuclease domain of the Cas9 protein is specialized for cutting one strand of the double helix; see, e.g., Jinek et al., “A programmable dual-RNA-guided DNA endonuclease in adaptive bacterial immunity, Science 337(6096): 816-821 (2012). Type III CRISPR-Cas systems include polymerase and RAMP modules. Type III systems can be further divided into sub-types III-A and III-B. Type III-A CRISPR-Cas systems have been shown to target plasmids, and the polymerase-like proteins of Type III-A systems are involved in the cleavage of target DNA; see, e.g., Marraffini et al., “CRISPR interference limits horizontal gene transfer in Staphylococci by targeting DNA,” Science 322(5909):1843-1845 (2008). Type III-B CRISPR-Cas systems have also been shown to target RNA; see, e.g., Hale et al., “RNA-guided RNA cleavage by a CRISPR-RNA-Cas protein complex,” Cell 139(5): 945-956 (2009). CRISPR-Cas systems include engineered and/or programmed nuclease systems derived from naturally accruing CRISPR-Cas systems. CRISPR-Cas systems may include engineered and/or mutated Cas proteins. CRISPR-Cas systems may include engineered and/or programmed guide RNA.

In some specific examples, the Cas protein in one of the present Cas-gRNA RNPs may include Cas9 or other suitable Cas that may cut the target polynucleotide at the sequence to which the gRNA is complementary, in a manner such as described in the following references, the entire contents of each of which are incorporated by reference herein: Nachmanson et al., “Targeted genome fragmentation with CRISPR/Cas9 enables fast and efficient enrichment of small genomic regions and ultra-accurate sequencing with low DNA input (CRISPR-DS),” Genome Res. 28(10): 1589-1599 (2018); Vakulskas et al., “A high-fidelity Cas9 mutant delivered as a ribonucleoprotein complex enables efficient gene editing in human hematopoietic stem and progenitor cells,” Nature Medicine 24: 1216-1224 (2018); Chatterjee et al., “Minimal PAM specificity of a highly similar SpCas9 ortholog,” Science Advances 4(10): eaau0766, 1-10 (2018); Lee et al., “CRISPR-Cap: multiplexed double-stranded DNA enrichment based on the CRISPR system,” Nucleic Acids Research 47(1): 1-13 (2019). Isolated Cas9-crRNA complex from the S. thermophilus CRISPR-Cas system as well as complex assembled in vitro from separate components demonstrate that it binds to both synthetic oligodeoxynucleotide and plasmid DNA bearing a nucleotide sequence complementary to the crRNA. It has been shown that Cas9 has two nuclease domains-RuvC- and HNH-active sites/nuclease domains, and these two nuclease domains are responsible for the cleavage of opposite DNA strands. In some examples, the Cas9 protein is derived from Cas9 protein of S. thermophilus CRISPR-Cas system. In some examples, the Cas9 protein is a multi-domain protein having about 1,409 amino acids residues.

In other examples, the Cas may be engineered so as not to cut the target polynucleotide at the sequence to which the gRNA is complementary, e.g., in a manner such as described in the following references, the entire contents of each of which are incorporated by reference herein: Guilinger et al., “Fusion of catalytically inactive Cas9 to Fokl nuclease improves the specificity of genome modification,” Nature Biotechnology 32: 577-582 (2014); Bhatt et al., “Targeted DNA transposition using a dCas9-transposase fusion protein,” https://doi.org/10.1101/571653, pages 1-89 (2019); Xu et al., “CRISPR-assisted targeted enrichment-sequencing (CATE-seq),” available at URL www.biorxiv.org/content/10.1101/672816v1, 1-30 (2019); and Tijan et al., “dCas9-targeted locus-specific protein isolation method identifies histone gene regulators,” PNAS 115(12): E2734-E2741 (2018). Cas that lacks nuclease activity may be referred to as deactivated Cas (dCas). In some examples, the dCas may include a nuclease-null variant of the Cas9 protein, in which both RuvC- and HNH-active sites/nuclease domains are mutated. A nuclease-null variant of the Cas9 protein (dCas9) binds to double-stranded DNA, but does not cleave the DNA. Another variant of the Cas9 protein has two inactivated nuclease domains with a first mutation in the domain that cleaves the strand complementary to the crRNA and a second mutation in the domain that cleaves the strand non-complementary to the crRNA. In some examples, the Cas9 protein has a first mutation D10A and a second mutation H840A.

In still other examples, the Cas protein includes a Cascade protein. Cascade complex in E. coli recognizes double-stranded DNA (dsDNA) targets in a sequence-specific manner. E. coli Cascade complex is a 405-kDa complex including five functionally essential CRISPR-associated (Cas) proteins (CasA1B2C6D1E1, also called Cascade protein) and a 61-nucleotide crRNA. The crRNA guides Cascade complex to dsDNA target sequences by forming base pairs with the complementary DNA strand while displacing the noncomplementary strand to form an R-loop. Cascade recognizes target DNA without consuming ATP, which suggests that continuous invader DNA surveillance takes place without energy investment; see, e.g., Matthijs et al., “Structural basis for CRISPR RNA-guided DNA recognition by Cascade,” Nature Structural & Molecular Biology 18(5): 529-536 (2011). In still other examples, the Cas protein includes a Cas3 protein. Illustratively, E. coli Cas3 may catalyze ATP-independent annealing of RNA with DNA forming R-loops, and hybrid of RNA base-paired into duplex DNA. Cas3 protein may use gRNA that is longer than that for Cas9; see, e.g., Howard et al., “Helicase disassociation and annealing of RNA-DNA hybrids by Escherichia coli Cas3 protein,” Biochem J. 439(1): 85-95 (2011). Such longer gRNA may permit easier access of other elements to the target DNA, e.g., access of a primer to be extended by polymerase. Another feature provided by Cas3 protein is that Cas3 protein does not require a PAM sequence as may Cas9, and thus provides more flexibility for targeting desired sequence. R-loop formation by Cas3 may utilize magnesium as a co-factor; see, e.g., Howard et al., “Helicase disassociation and annealing of RNA-DNA hybrids by Escherichia coli Cas3 protein,” Biochem J. 439(1): 85-95 (2011). Cas9 variants also have been developed that reduce or avoid the need for PAM sequences; see, e.g., Walton et al., “Unconstrained genome targeting with near-PAMless engineered CRISPR-Cas9 variants,” Science 368(6488): 290-296 (2020), the entire contents of which are incorporated by reference herein. It will be appreciated that any suitable cofactors, such as cations, may be used together with the Cas proteins used in the present compositions and methods.

It also should be appreciated that any CRISPR-Cas systems capable of disrupting the double stranded polynucleotide and creating a loop structure may be used. For example, the Cas proteins may include, but are not limited to, Cas proteins such as described in the following references, the entire contents of each of which are incorporated by reference herein: Haft et al., “A guild of 45 CRISPR-associated (Cas) protein families and multiple CRISPR/Cas subtypes exist in prokaryotic genomes,” PLoS Comput Biol. 1(6): e60, 1-10 (2005); Zhang et al., “Expanding the catalog of cas genes with metagenomes,” Nucl. Acids Res. 42(4): 2448-2459 (2013); and Strecker et al., “RNA-guided DNA insertion with CRISPR-associated transposases,” Science 365(6448): 48-53 (2019) in which the Cas protein may include Cas12k. Some of these CRISPR-Cas systems may utilize a specific sequence to recognize and bind to the target sequence. For example, Cas9 may utilize the presence of a 5′-NGG protospacer-adjacent motif (PAM).

In some examples, the Cas protein may be selected so as to leave a single-stranded DNA overhang region following dsDNA cleavage, e.g., of one or more bases, illustratively 2-5 bases. For example, CRISPR-Cas12a (Cpf1) is commercially available from Integrated DNA Technologies, Inc. (Coralville, Iowa). According to the manufacturer, CRISPR-Cas12a (Cpf1) produces a staggered cut with a 5′ overhang, and may target different sites than CRISPR-Cas9. In some examples, the 5′ overhang may be 5 bases long. Some of these CRISPR-Cas systems may utilize a PAM. For example, Cas12a (Cpf1 or C2c1) or FnCas12a may use a PAM of TTTN upstream of the cleavage site, while emerging Cas12a orthologs may have a reduced PAM requirement (e.g., YTN), in a manner such as described in Teng et al., “Enhanced mammalian genome editing by new Cas12a orthologs with optimized crRNA scaffolds,” Genome Biology 20: 15 (2019), the entire contents of which are incorporated by reference herein. Cas12 may be derived from organisms such as Francisella novicida, Acidaminococcus sp., Lachnospiraceae sp., and Prevotella sp. For further details regarding Cas12a, see Covsky et al., “CRISPR-Cas12a exploits R-loop asymmetry to form double-strand breaks,” eLife, 9: e55143 (2020), the entire contents of which are incorporated by reference herein.

CRISPR-Cas systems may also include engineered and/or programmed guide RNA (gRNA). As used herein, the terms “guide RNA” and “gRNA” (and sometimes referred to in the art as single guide RNA, or sgRNA) is intended to mean RNA including a sequence that is complementary or substantially complementary to a region of a target DNA sequence and that guides a Cas protein to that region. A guide RNA may include nucleotide sequences in addition to that which is complementary or substantially complementary to the region of a target DNA sequence. Methods for designing gRNA are well known in the art, and nonlimiting examples are provided in the following references, the entire contents of each of which are incorporated by reference herein: Stevens et al., “A novel CRISPR/Cas9 associated technology for sequence-specific nucleic acid enrichment,” PLoS ONE 14(4): e0215441, pages 1-7 (2019); Fu et al., “Improving CRISPR-Cas nuclease specificity using truncated guide RNAs, Nature Biotechnology 32(3): 279-284 (2014); Kocak et al., “Increasing the specificity of CRISPR systems with engineered RNA secondary structures,” Nature Biotechnology 37: 657-666 (2019); Lee et al., “CRISPR-Cap: multiplexed double-stranded DNA enrichment based on the CRISPR system,” Nucleic Acids Research 47(1): e1, 1-13 (2019); Quan et al., “FLASH: a next-generation CRISPR diagnostic for multiplexed detection of antimicrobial resistance sequences,” Nucleic Acids Research 47(14): e83, 1-9 (2019); and Xu et al., “CRISPR-assisted targeted enrichment-sequencing (CATE-seq),” https://doi.org/10.1101/672816, 1-30 (2019).

In some examples, gRNA includes a chimera, e.g., CRISPR RNA (crRNA) fused to trans-activating CRISPR RNA (tracrRNA). Such a chimeric single-guided RNA (sgRNA) is described in Jinek et al., “A programmable dual-RNA-guided endonuclease in adaptive bacterial immunity,” Science 337 (6096): 816-821 (2012). The Cas protein may be directed by a chimeric sgRNA to any genomic locus followed by a 5′-NGG protospacer-adjacent motif (PAM). In one nonlimiting example, crRNA and tracrRNA may be synthesized by in vitro transcription, using a synthetic double stranded DNA template including the T7 promoter. The tracrRNA may have a fixed sequence, whereas the target sequence may dictate part of the crRNA's sequence. Equal molarities of crRNA and tracrRNA may be mixed and heated at 55° C. for 30 seconds. Cas9 may be added at the same molarity at 37° C. and incubated for 10 minutes with the RNA mix. A 10-20 fold molar excess of the resulting Cas9-gRNA RNP then may be added to the target DNA. The binding reaction may occur within 15 minutes. Other suitable reaction conditions readily may be used.

As used herein, the terms “fusion protein” and “chimeric protein” are intended to mean an element that includes two or more polypeptide domains with different functional properties (such as different enzymatic activities) than one another. The domains may be coupled to one another covalently or non-covalently. Fusion proteins may optionally include a third, fourth or fifth or other polypeptide domains operatively linked to one or more other of the polypeptide domains. Fusion proteins may include multiple copies of the same polypeptide domain. Fusion proteins may also or alternatively include one or more mutations in one or more of the polypeptides. A fusion protein may include one or more non-protein elements, such as a polynucleotide (illustratively, gRNA) and/or a linker that couples the domains to one another. For nonlimiting examples of a fusion protein, see the following references, the entire contents of which are incorporated by reference herein: Guilinger et al., “Fusion of catalytically inactive Cas9 to Fokl nuclease improves the specificity of genome modification,” Nature Biotechnology 32: 577-582 (2014); Bhatt et al., “Targeted DNA transposition using a dCas9-transposase fusion protein,” https://doi.org/10.1101/571653, pages 1-89 (2019); and Strecker et al., “RNA-guided DNA insertion with CRISPR-associated transposases,” Science 365(6448): 48-53 (2019). Another example fusion protein is ShCAST (Scytonema hofmanni CRISPR associated transposase), which includes Cas12k and a Tn7-like transposase. For further details regarding ShCAST, including the Cas12k and Tn7 therein, see Strecker et al., “RNA-Guided DNA insertion with CRISPR-associated transposases,” Science 365(6448): 48-53 (2019), the entire contents of which are incorporated by reference herein.

As used herein, the term “transposase” is intended to mean an enzyme capable of coupling an oligonucleotide to a polynucleotide. In some examples, the oligonucleotide may include an amplification adapter, and optionally may include a unique molecular identifier (UMI). A transposase may cut the polynucleotide while adding the oligonucleotide thereto. One nonlimiting example of a transposase is Tn5. In still further examples, transposases may include integrases from retrotransposons or retroviruses. Transposases, transposons and transposon complexes are generally known to those of skill in the art, as exemplified by the disclosure of US 2010/0120098, the entire contents of which are incorporated by reference herein.

For additional nonlimiting examples of transposases that may be used in a manner such as provided herein, see the following references, the entire contents of each of which are incorporated by reference herein: Strecker et al., “RNA-guided DNA insertion with CRISPR-associated transposases,” Science 365(6448): 48-53 (2019); Klompe et al., “Transposon-encoded CRISPR-Cas systems direct RNA-guided DNA integration,” Nature 571: 219-225 (2019); and Bhatt et al., “Targeted DNA transposition using a dCas9-transposase fusion protein,” https://doi.org/10.1101/571653, pages 1-89 (2019). Other examples of known transposition systems that could be used in the provided methods include, but are not limited to, Staphylococcus aureus Tn552, Ty1, Transposon Tn7, Tn/O and IS10, Mariner transposase, Tel, P Element, Tn3, bacterial insertion sequences, retroviruses, and retrotransposon of yeast (see, e.g., Colegio et al., 2001, J Bacteriol. 183: 2384-8; Kirby et al., 2002, Mol. Microbiol. 43: 173-86; Devine and Boeke, 1994, Nucleic Acids Res., 22: 3765-72; International Patent Application No. WO 95/23875; Craig, 1996, Science 271: 1512; Craig, 1996, Review in: Curr Top Microbiol Immunol. 204: 27-48; Kleckner et al., 1996, Curr Top Microbiol Immunol. 204: 49-82; Lampe et al., 1996, EMBO J 15: 5470-9; Plasterk, 1996, Curr Top Microbiol Immunol 204: 125-43; Gloor, 2004, Methods Mol. Biol. 260: 97-114; Ichikawa and Ohtsubo, 1990, J Biol. Chem. 265: 18829-32; Ohtsubo and Sekine, 1996, Curr. Top. Microbiol. Immunol. 204: 1-26; Brown et al., 1989, Proc Natl Acad Sci USA 86: 2525-9; and Boeke and Corces, 1989, Annu Rev Microbiol. 43: 403-34). As another example, ShCAST (Scytonema hofmanni CRISPR associated transposase) includes a Tn7-like transposase; for further details, see Strecker et al., “RNA-Guided DNA insertion with CRISPR-associated transposases,” Science 365(6448): 48-53 (2019), the entire contents of which are incorporated by reference herein.

In some examples, a transposase may perform a process that may be referred to as “tagmentation” or “transposition” that results in fragmentation of the target polynucleotide and ligation of adapters to the 5′ end of both strands of double-stranded DNA fragments, or to the 5′ and 3′ ends, e.g., in a manner such as described in U.S. 2010/0120098 or in WO 2010/04860, the entire contents of each of which are incorporated by reference herein.

A transposase may form a “transposition complex” that includes the transposase, a transposon end-including composition, and a double-stranded polynucleotide, and may catalyze insertion or transposition of the transposon end-including composition into the double-stranded target polynucleotide. Example transposition complexes include, but are not limited to, those formed by a hyperactive Tn5 transposase and a Tn5-type transposon end or by a MuA transposase and a Mu transposon end including R1 and R2 end sequences; see, e.g., the following references, the entire contents of each of which are incorporated by reference herein: Goryshin et al., “Tn5 in vitro transposition,” J. Biol. Chem. 273: 7367-7394 (1998); Mizuuchi, “In vitro transposition of bacteriophage Mu: a biochemical approach to a novel replication reaction,” Cell 35 (3 pt 2): 785-794 (1983); and Savilahti et al., “The phage Mu transposomes core: DNA requirements for assembly and function,” EMBO J. 14(19): 4893-4903 (1995). The combination of a transposase and transposon end may be referred to as a “transposome.”

Still further examples of transposases and other suitable transposition systems include Staphylococcus aureus Tn552 (see, e.g., Colegio et al., “In vitro transposition system for efficient generation of random mutants of Campylobacter jejuni,” J Bacteriol. 183: 2384-2388 (2001) and Kirby et al., “Cryptic plasmids of Mycobacterium avium: Tn552 to the rescue,” Mol Microbiol., 43(1): 173-186 (2002)); TyI (Devine et al., “Efficient integration of artificial transposons into plasmid targets in vitro: a useful tool for DNA mapping, sequencing and genetic analysis,” Nucleic Acids Res. 22(18): 3765-3772 (1994) and International Patent Application No. WO 95/23875); Transposon Tn7 (Craig, “V(D)J recombination and transposition: Closer than expected,” Science 271(5255): 1512 (1996) and Craig, Review in: Curr Top Microbiol Immunol, 204: 27-48 (1996)); TnIO and ISlO (Kleckner et al., Curr Top Microbiol Immunol, 204: 49-82 (1996)); Mariner transposase (Lampe et al., “A purified mariner transposase is sufficient to mediate transposition in vitro,” EMBO J. 15(19): 5470-5479 (1996)); Tci (Plasterk, Curr Top Microbiol Immunol, 204: 125-143 (1996)), P Element (Gloor, “Gene targeting in Drosophila,” Methods Mol Biol 260: 97-114 (2004)); TnJ (Ichikawa et al., “In vitro transposition of transposon Tn3,” J Biol Chem. 265(31): 18829-18832 (1990)); bacterial insertion sequences (Ohtsubo et al., “Bacterial insertion sequences,” Curr. Top. Microbiol. Immunol. 204:1-26 (1996)); retroviruses (Brown et al., “Retroviral integration: Structure of the initial covalent product and its precursor, and a role for the viral IN protein,” Proc Natl Acad Sci USA, 86: 2525-2529 (1989)); and retrotransposon of yeast (Boeke et al., “Transcription and reverse transcription of retrotransposons,” Annu Rev Microbiol. 43: 403-434 (1989).

As used herein, the term “nuclease” is intended to mean an enzyme capable of cleaving the phosphodiester bonds between the nucleotide subunits of polynucleotides. The term “endonuclease” refers to an enzyme capable of cleaving the phosphodiester bond within a polynucleotide chain.

As used herein, the term “nickase” refers to an endonuclease which cleaves only a single strand of a DNA duplex. Some CRISPR-Cas systems may cleave only one strand of a double-stranded polynucleotide, and accordingly may be referred to as CRISPR nickases or as Cas-gRNA RNP nickases. For example, the term “Cas9 nickase” refers to a nickase derived from a Cas9 protein, typically by inactivating one nuclease domain of Cas9 protein. Nonlimiting examples of CRISPR nickases include S. Pyogenes Cas9 with a first mutation D10A and a second mutation H840A.

In the context of a polypeptide, the terms “variant” and “derivative” as used herein refer to a polypeptide that includes an amino acid sequence of a polypeptide or a fragment of a polypeptide, which has been altered by the introduction of amino acid residue substitutions, deletions or additions. A variant or a derivative of a polypeptide can be a fusion protein which contains part of the amino acid sequence of a polypeptide. The term “variant” or “derivative” as used herein also refers to a polypeptide or a fragment of a polypeptide, which has been chemically modified, e.g., by the covalent attachment of any type of molecule to the polypeptide. For example, but not by way of limitation, a polypeptide or a fragment of a polypeptide can be chemically modified, e.g., by glycosylation, acetylation, pegylation, phosphorylation, amidation, derivatization by known protecting/blocking groups, proteolytic cleavage, linkage to a cellular ligand or other protein, etc. The variants or derivatives are modified in a manner that is different from naturally occurring or starting peptide or polypeptides, either in the type or location of the molecules attached. Variants or derivatives further include deletion of one or more chemical groups which are naturally present on the peptide or polypeptide. A variant or a derivative of a polypeptide or a fragment of a polypeptide can be chemically modified by chemical modifications using techniques known to those of skill in the art, including, but not limited to specific chemical cleavage, acetylation, formulation, metabolic synthesis of tunicamycin, etc. Further, a variant or a derivative of a polypeptide or a fragment of a polypeptide can contain one or more non-classical amino acids. A polypeptide variant or derivative may possess a similar or identical function as a polypeptide or a fragment of a polypeptide described herein. A polypeptide variant or derivative may possess an additional or different function compared with a polypeptide or a fragment of a polypeptide described herein.

As used herein, the term “sequencing” is intended to mean determining the sequence of a polynucleotide. Sequencing may include one or more of sequencing-by-synthesis, bridge PCR, chain termination sequencing, sequencing by hybridization, nanopore sequencing, and sequencing by ligation.

As used herein, the term “dehosting” is intended to mean the selective deactivation or degradation of polynucleotides of one species relative to the polynucleotides of another species. For example, a first species such as a mammal (e.g., a human) may act as a host to numerous other species, such as bacteria, fungi, and viruses. It may be desirable to selectively deactivate or degrade the polynucleotides of the first species so that the polynucleotides of one or more other species may be amplified and sequenced.

As used herein, to be “selective” for an element is intended to mean to couple to that target and not to couple to a different element. For example, a Cas-gRNA RNP that is selective for a species specific repetitive element may couple to that species specific repetitive element and not to a different species specific repetitive element.

As used herein, the term “species specific repetitive element” is intended to mean a repeating sequence that occurs within the polynucleotides of a given species and that may not occur within the polynucleotides of another species. A species having multiple chromosomes (such as mammal, e.g., human) may include different species specific elements on each chromosome, or may include the same species specific element on each chromosome, or a mixture of same and different species specific elements on each chromosome. One example of a species specific repetitive element is a photospacer adjacent motif, or PAM sequence, such as NGG. The gRNA of a Cas-gRNA RNP may have a sequence that hybridizes to a species specific repetitive element.

As used herein, the terms “unique molecular identifier” and “UMI” are intended to mean an oligonucleotide that may be coupled to a polynucleotide and via which the polynucleotide may be identified. For example, a set of different UMIs may be coupled to a plurality of different polynucleotides, and each of those polynucleotides may be identified using the particular UMI coupled to that polynucleotide.

As used herein, the term “whole genome” or “WG” of a species is intended to mean a set of one or more polynucleotides that, together, provide the majority of polynucleotides used by the cellular processes of that species. The whole genome of a species may include any suitable combination of the species' chromosomal DNA and/or mitochondrial DNA, and in the case of a plant species may include the DNA contained in the chloroplast. The set of one or more polynucleotides together may provide at least about 50%, or at least about 60%, or at least about 70%, or at least about 80%, or at least about 90%, or at least about 95%, or at least about 98%, or at least about 99%, of the polynucleotides used by the cellular processes of that species.

As used herein, the term “fragment” is intended to mean a portion of a polynucleotide. For example, a polynucleotide may be a total number of bases long, and a fragment of that polynucleotide may be less than the total number of bases long.

As used herein, the term “sample” is intended to mean a volume of fluid that includes one or more polynucleotides. The polynucleotide(s) in sample may include a whole genome, or may include only a portion of a whole genome. A sample may include polynucleotides from a single species, or from multiple species.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” include a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies. That is, the individual antibodies including the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art. Monoclonal antibodies, as the term is used herein, may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, terms such as “target specific” and “selective,” when used in reference to a guide RNA or other polynucleotide, are intended to mean a polynucleotide that includes a sequence that is specific to (substantially complementary to and may hybridize to) a sequence within another polynucleotide.

As used herein, the terms “complementary” and “substantially complementary,” when used in reference to a polynucleotide, are intended to mean that the polynucleotide includes a sequence capable of selectively hybridizing to a sequence in another polynucleotide under certain conditions.

As used therein, terms such as “amplification” and “amplify” refer to the use of any suitable amplification method to generate amplicons of a polynucleotide. Polymerase chain reaction (PCR) is one nonlimiting amplification method. Other suitable amplification methods known in the art include, but are not limited to, rolling circle amplification; riboprimer amplification (e.g., as described in U.S. Pat. No. 7,413,857); ICAN; UCAN; ribospia; terminal tagging (e.g., as described in U.S. 2005/0153333); and Eberwine-type aRNA amplification or strand-displacement amplification. Additional, nonlimiting examples of amplification methods are described in WO 02/16639; WO 00/56877; AU 00/29742; U.S. Pat. Nos. 5,523,204; 5,536,649; 5,624,825; 5,631,147; 5,648,211; 5,733,752; 5,744,311; 5,756,702; 5,916,779; 6,238,868; 6,309,833; 6,326,173; 5,849,547; 5,874,260; 6,218,151; 5,786,183; 6,087,133; 6,214,587; 6,063,604; 6,251,639; 6,410,278; WO 00/28082; U.S. Pat. Nos. 5,591,609; 5,614,389; 5,773,733; 5,834,202; 6,448,017; 6,124,120; and 6,280,949.

The terms “polymerase chain reaction” and “PCR,” as used herein, refer to a procedure wherein small amounts of a polynucleotide, e.g., RNA and/or DNA, are amplified. Generally, amplification primers are coupled to the polynucleotide for use during the PCR. See, e.g., the following references, the entire contents of which are incorporated by reference herein: U.S. Pat. No. 4,683,195 to Mullis; Mullis et al., Cold Spring Harbor Symp. Quant. Biol., 51: 263 (1987); and Erlich, ed., PCR Technology, (Stockton Press, N Y, 1989). A wide variety of enzymes and kits are available for performing PCR as known by those skilled in the art. For example, in some examples, the PCR amplification is performed using either the FAILSAFE™ PCR System or the MASTERAMP™ Extra-Long PCR System from EPICENTRE Biotechnologies, Madison, Wis., as described by the manufacturer.

As used herein, terms such as “ligation” and “ligating” are intended to mean to form a covalent bond or linkage between the termini of two or more polynucleotides. The nature of the bond or linkage may vary widely and the ligation may be carried out enzymatically or chemically. Ligations may be carried out enzymatically to form a phosphodiester linkage between a 5′ carbon terminal nucleotide of one oligonucleotide with a 3′ carbon of another nucleotide. Template driven ligation reactions are described in the following references, the entire contents of each of which are incorporated by reference herein: U.S. Pat. Nos. 4,883,750; 5,476,930; 5,593,826; and 5,871,921. Ligation also may be performed using non-enzymatic formation of phosphodiester bonds, or the formation of non-phosphodiester covalent bonds between the ends of polynucleotides, such as phosphorothioate bonds, disulfide bonds, and the like.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

### Compositions and Methods for Cas-gRNA RNP Mediated Dehosting

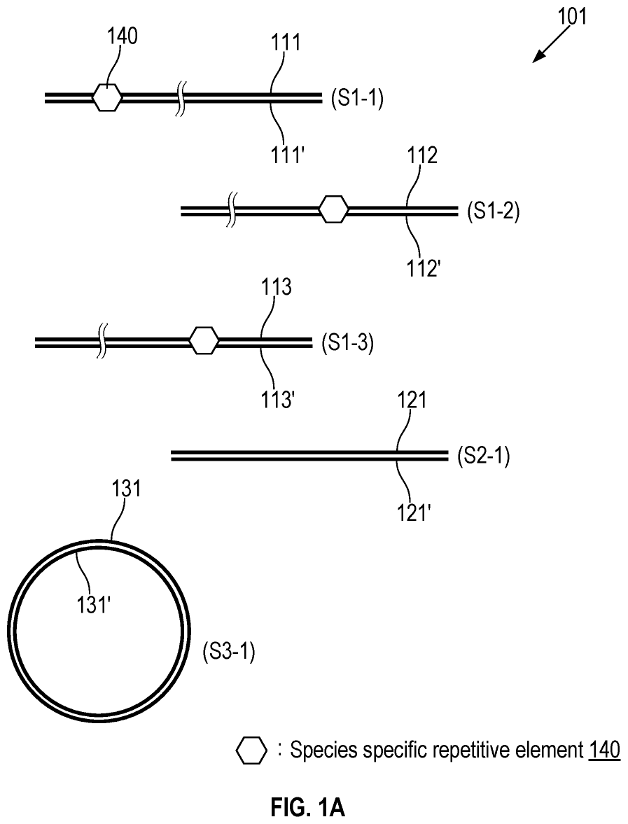

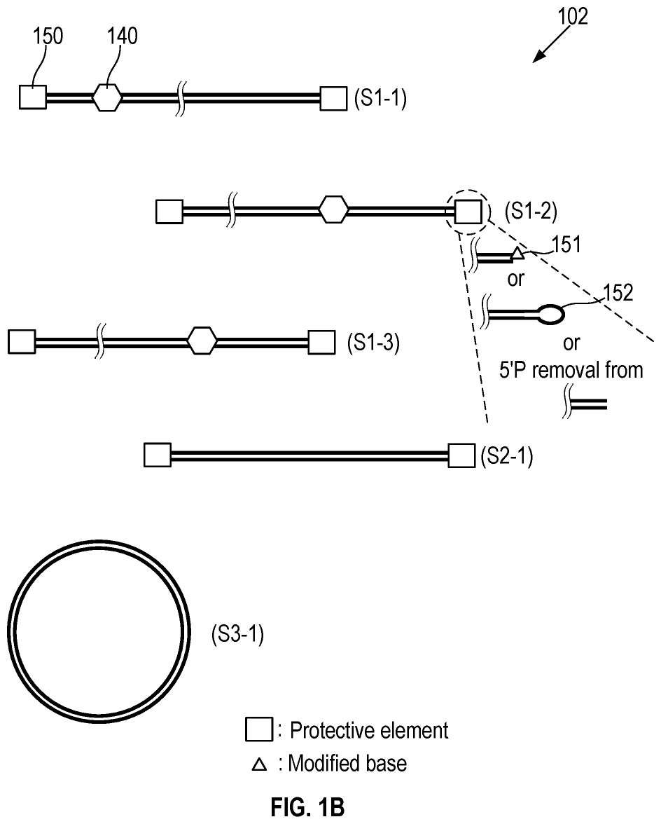

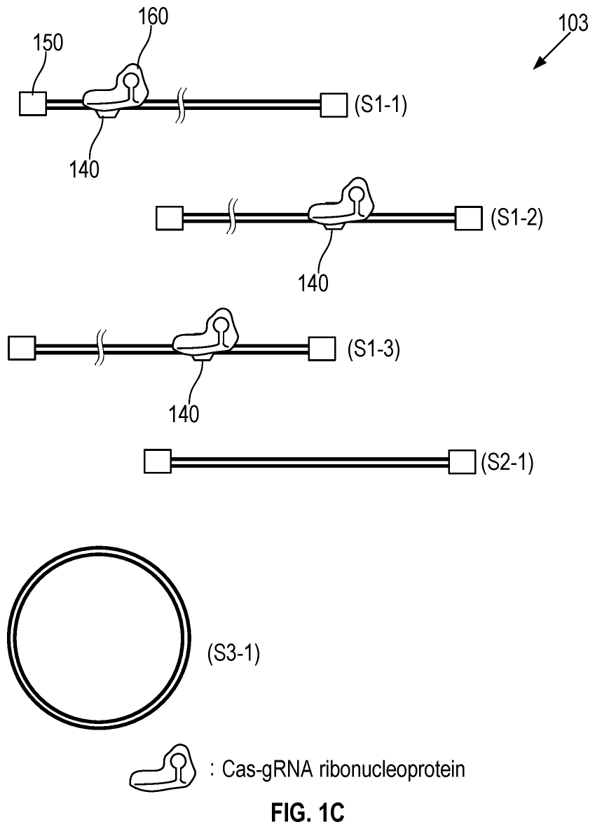

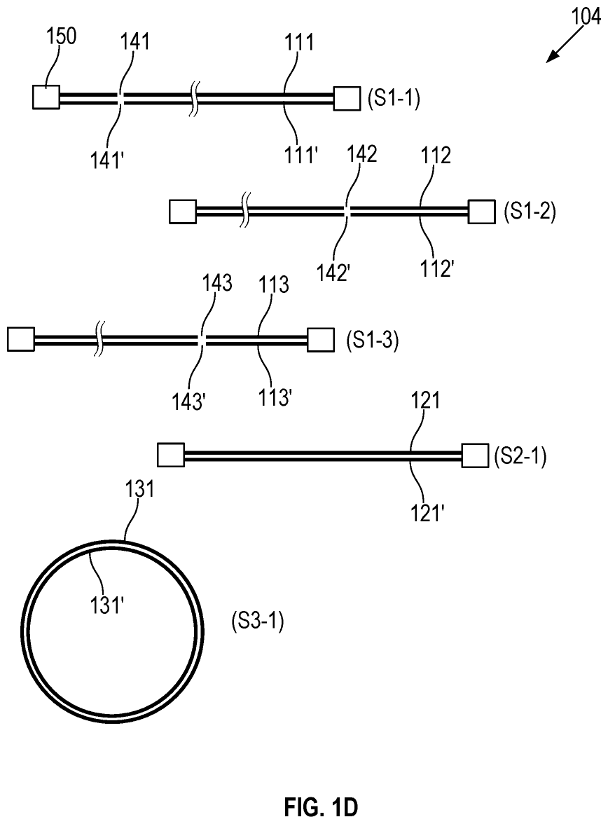

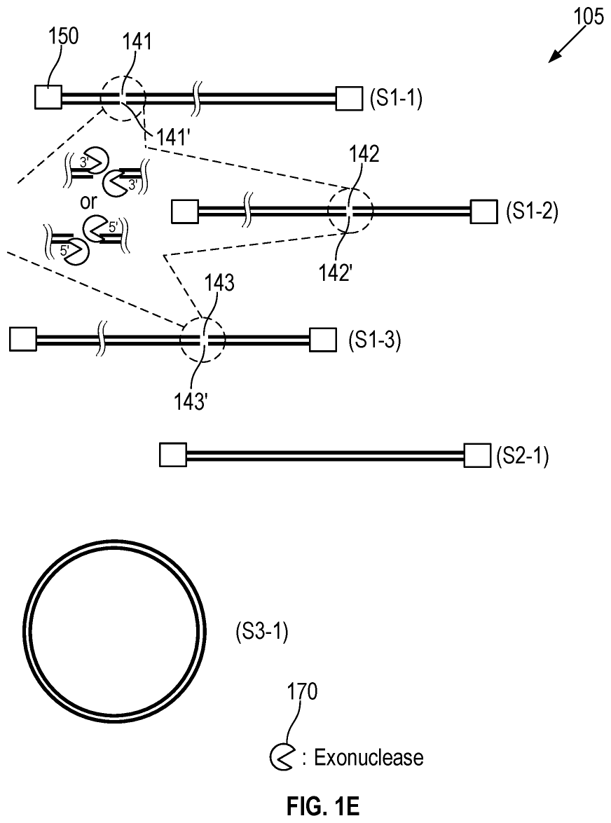

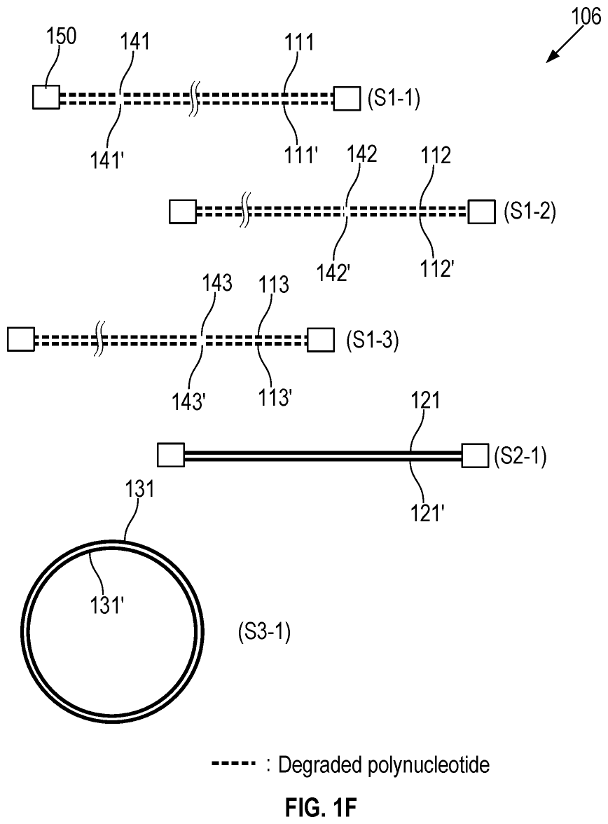

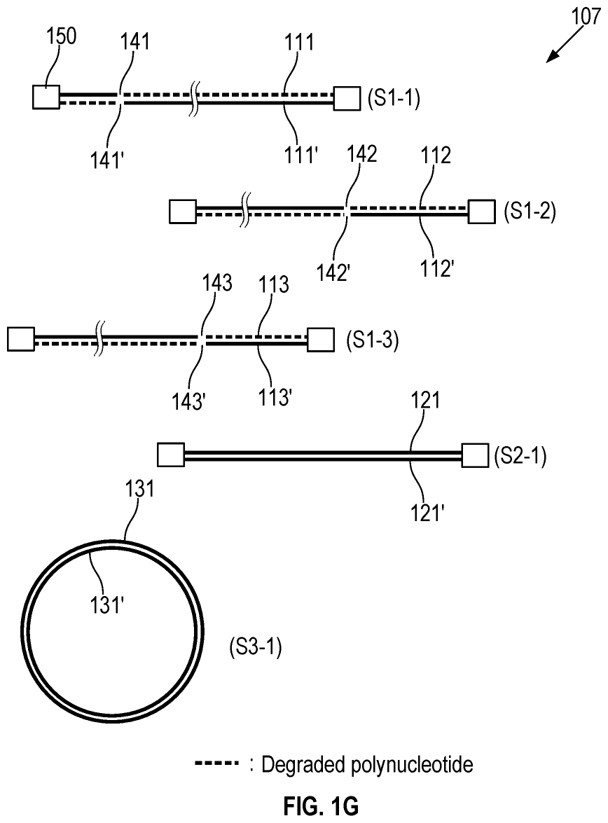

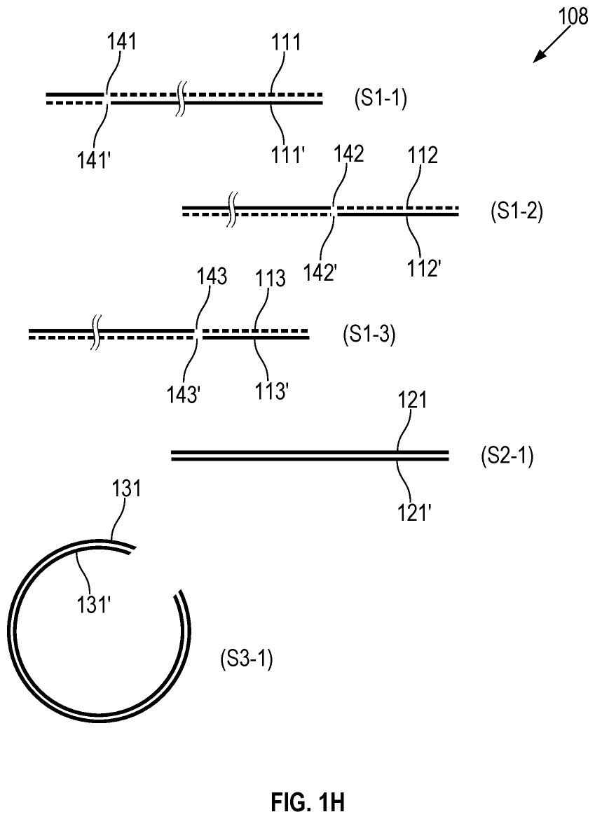

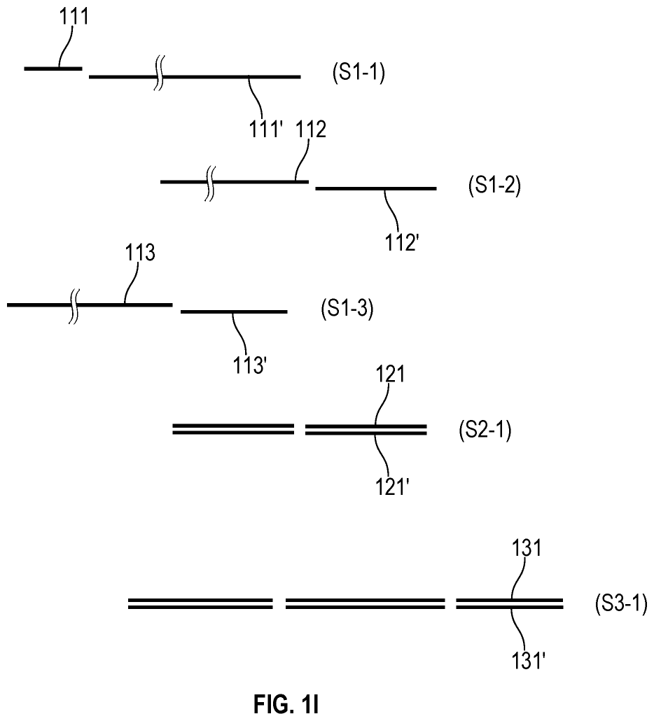

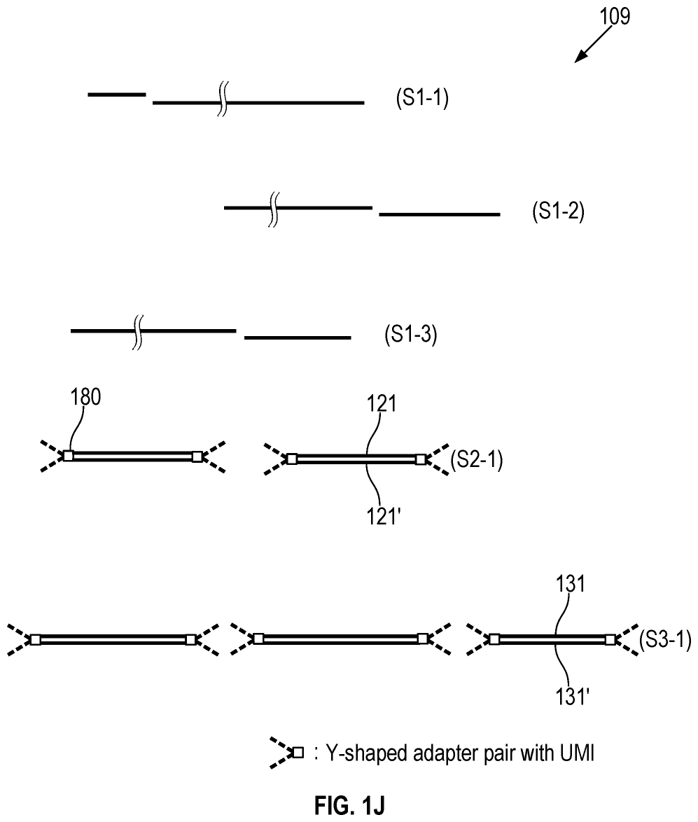

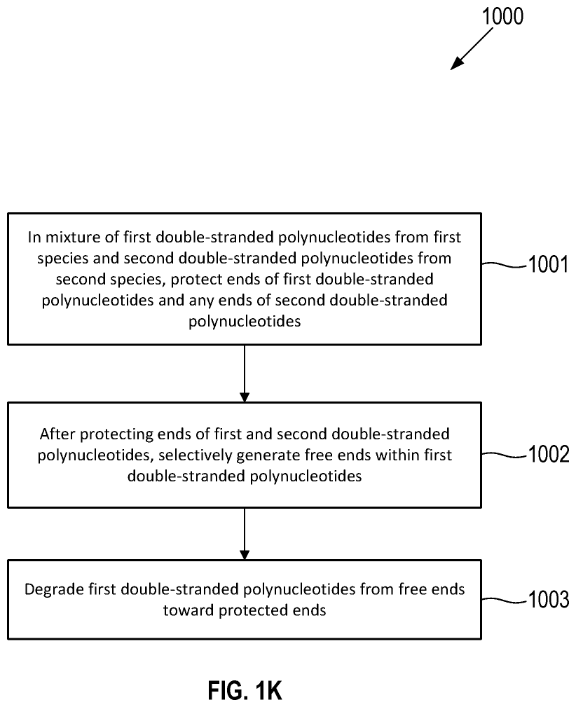

Some examples herein relate to Cas-gRNA RNP mediated dehosting. For example, FIGS. 1A-1K schematically illustrate example compositions and operations in a process flow for Cas-gRNA RNP mediated dehosting.

Species that are more complex, illustratively mammals, may host a plurality of other, simpler species such as bacteria, fungi, and viruses. It can be desirable to sequence the polynucleotides (such as DNA) of species that are being hosted, but it can be difficult to sufficiently separate such polynucleotides from that of the host species. For example, a sample of purified polynucleotides from fluid or tissue from the host primarily may include polynucleotides from the host (e.g., about 99% or more), and a relatively low amount of polynucleotides from other species (e.g., about 1% or less). As such, sequencing that sample primarily may yield the sequence of the host, with relatively little information about the sequence of the other species. As provided herein, the polynucleotides of a given species (such as a host) may be removed from a sample in such a manner as to enhance the ability to sequence the polynucleotides of one or more other species within that sample.

For example, as shown in FIG. TA, a sample obtained from a first species may include a mixture of first double-stranded polynucleotides from a first species and second double-stranded polynucleotides from one or more second species. Illustratively, the first species (S1) may be a mammal (e.g., a human), which may act as a host to numerous other species, such as bacteria, fungi, and viruses (S2, S3, and so on). In the nonlimiting example shown in FIG. TA, composition 101 includes a mixture of polynucleotides S1-1, S1-2, S1-3 from the first species; a polynucleotide S2-1 from a second species; and a polynucleotide S3-1 from a third species. Each of the polynucleotides from the first species S1-1, S1-2, S1-3 from the first species may include a species specific repetitive element 140 such as illustrated in FIG. 1A. For example, where the first species is mammalian, the polynucleotides from that species may include a mammalian specific repetitive element. For example, where the first species is human, each of the polynucleotides from that human may include one or more human specific repetitive elements 140.

It will be appreciated that the concentration, number, and type of polynucleotides from each given species may vary for each particular sample. For example, if the first species is a host to the second and third species, the sample may contain a significantly higher concentration of polynucleotides from the first species than the second and third species. Additionally, the first species may have greater genetic complexity, e.g., may include a genome with multiple polynucleotides, such as twenty-three relatively long chromosomes S1-1, S1-2, S1-3 . . . S1-23 for a human, while the second and/or third species may be genetically simpler and may, for example, include a genome with only a single, relatively short polynucleotide. Additionally, the polynucleotide(s) of one or more species in the mixture may be fragmented ex vivo into shorter pieces than those species would typically use during normal physiological processes in vivo. Additionally, the polynucleotide(s) of one or more species in the mixture may be circular (such as S3-1) and thus may not have any ends.

As illustrated in FIG. 1A, each of the polynucleotides in the mixture may be double-stranded. For example, polynucleotide S1-1 may include first strand 111 and complementary second strand 111′; polynucleotide S1-2 may include first strand 112 and complementary second strand 112′; polynucleotide S1-3 may include first strand 113 and complementary second strand 113′; polynucleotide S2-1 may include first strand 121 and complementary second strand 121′; and polynucleotide S3-1 may include first strand 131 and complementary second strand 131′. In some examples, the double-stranded polynucleotides from the first, second, and/or third species may include double-stranded DNA.

Ends of the first double-stranded polynucleotides and the ends, if any, of the second double-stranded polynucleotides, may be protected. For example, as illustrated in FIG. 1B, composition 102 includes protective elements 150 that protect any ends of double-stranded polynucleotides in the mixture. Illustratively, protective elements 150 are coupled to, and protect, the ends of polynucleotides S1-1, S1-2 and S1-3 of the first species and the ends of polynucleotide S2-1 of the second species. Because polynucleotide S3-1 of the third species is circular, such polynucleotide may not have any end(s) to which protective elements 150 may be coupled. Protective elements 150 may include any suitable chemical moiety that inhibits action of one or more enzymes (such as an exonuclease) upon the ends of the double-stranded polynucleotides to which such protective elements are coupled. For example, as illustrated in the inset of FIG. 1B, protective elements 150 may include modified bases 151, hairpin adapters 152 that are ligated to the ends, or 5′-dephosphorylated ends. Modified bases 151 may, for example, include phosphorothioate bonds or 3′ phosphates, and may be added using a terminal transferase. Hairpin adapters 152 may include oligonucleotides including stem sequences that hybridize to one another and a loop sequence that extends between the stem sequences, and may be added in a manner such as known in the art, example performing end repair to fill in any overhangs, then adding an A overhang (“A-tail”) (e.g., using an exonuclease such as Klenow Fragment exo-), and then ligating hairpin adapters 152 to the end. The 5′ ends of the double stranded polynucleotides may be dephosphorylated using a suitable phosphatase enzyme.

After protecting the ends of the first and second double-stranded polynucleotides, free ends within the first double-stranded polynucleotides may be selectively generated. For example, FIG. 1C illustrates composition 103 in which Cas-gRNA RNPs 160 are hybridized to sequences that are present within the first double-stranded polynucleotides and that are not present within the second double-stranded polynucleotides, e.g., to species specific repetitive elements 140. The sequences then may be cut with the Cas-gRNA RNPs to generate the free ends in a manner such as illustrated in FIG. 1D, including composition 104 in which free ends 141, 141′ are generated in the strands of polynucleotide S1-1, free ends 142, 142′ are generated in the strands of polynucleotide S1-2, and free ends 143, 143′ are generated in the strands of polynucleotide S1-3, but free ends are not generated in polynucleotides S2-1 and S3-1 because those polynucleotides did not include the species specific repetitive elements 150 to which Cas-gRNA RNPs 160 selectively hybridize. The Cas may include, for example, Cas9.

The first double-stranded polynucleotides then may be degraded from the free ends, which were generated by Cas-gRNA RNPs 160, toward the protected ends. For example, composition 105 illustrated in FIG. 1E includes exonucleases 170 for degrading the first double-stranded polynucleotides S1-1, S1-2, S1-3. Any suitable exonucleases 170 may be used. Illustratively, the free ends may include 3′ ends in a manner such as shown in the upper portion of the inset of FIG. 1E, and the first double-stranded polynucleotides S1-1, S1-2, S1-3 may be degraded using exonuclease III. As another purely illustrative example, the free ends may include 5′ ends in a manner such as shown in the lower portion of the inset of FIG. 1E, and one strand of each of the first double-stranded polynucleotides S1-1, S1-2, S1-3 may be degraded using Lambda exonuclease. Depending on the particular type of protective elements 150 used, the use of the exonuclease may result in composition 106 illustrated in FIG. 1F in which both strands of each of polynucleotides S1-1, S1-2, S1-3 are degraded, or in composition 107 illustrated in FIG. 1G in which polynucleotides S1-1, S1-2, S1-3 are rendered single stranded. Illustratively, if protective elements 150 include hairpin oligonucleotides, then after degrading one strand the exonuclease may follow the hairpin to degrade the other strand, resulting in degradation of both strands. As another example, if protective elements 150 include modified bases or 5′-dephosphorylated bases, then after the exonuclease degrades one strand the protective element may inhibit the exonuclease from degrading the other strand. Regardless of the particular exonuclease used and whether the first species' polynucleotides are entirely degraded or are rendered single-stranded, polynucleotides S2-1 and S3-1 may not be degraded by that exonuclease because the ends of polynucleotide S2-1 are protected by protective element 150, and polynucleotide3 S3-1 lacks ends.

Following degradation of the first species' polynucleotides, amplification adapters may be ligated to the ends of any remaining double-stranded polynucleotides in the mixture. For example, FIG. 1H illustrates a composition 108 in which polynucleotides S1-1, S1-2, and S1-3 are degraded (e.g., both strands are degraded as illustrated in FIG. 1F, or the polynucleotides are rendered single-stranded as illustrated in FIGS. 1G and 1H), and in which protective groups 150 are removed from any remaining double-stranded polynucleotides in the mixture, e.g., from polynucleotide S2-1. Any remaining protective groups 150 coupled to any remaining portions of first species' polynucleotides may be removed as well. As illustrated in FIG. 1I, any circular polynucleotides (e.g., S3-1 of the third species) may be opened up, for example using tagmentation, shearing, or other suitable fragmentation technique, which also may fragment any remaining double-stranded polynucleotides in the mixture, e.g., S2-1. Amplification adapters then may be ligated to the remaining double-stranded polynucleotides, e.g., those of the second and third species, or the remaining double-stranded polynucleotides may be tagmented, to obtain composition 109 illustrated in FIG. 1J. Composition 109 includes, from the first species, substantially only single-stranded polynucleotides S1-1, S1-2, S1-3; from the second and/or third species, substantially only double-stranded polynucleotides S2-1, S3-1; and amplification adapters 180 ligated to ends of fragments of the second double-stranded polynucleotides S2-1, S3-1 and substantially not ligated to any ends of the first double-stranded polynucleotides S1-1, S1-2, S1-3. It will be appreciated that if the first species' polynucleotides are completely degraded in a manner such as described with reference to FIG. 1F, then composition 109 instead may not include any polynucleotides from the first species. In a manner such as illustrated in FIG. 1J, amplification adapters 180 may be Y-shaped and may include unique molecular identifiers (UMIs) such as described in the following references, the entire contents of each of which are incorporated by reference herein: Kennedy et al., “Detecting ultralow-frequency mutations by Duplex Sequencing,” Nat Protoc. 9: 2586-2606 (2014); and Kivioja et al., “Counting absolute numbers of molecules using unique molecular identifiers,” Nature Methods 9:72-42 (2012). Double stranded polynucleotides S2-1 and S3-1 subsequently may be amplified (e.g., using PCR) and sequenced, substantially without sequencing any of the polynucleotides from the first species. As such, the sequences of polynucleotides S2-1 and S3-1 may be obtained with relatively low, or even substantially no, background signal from the first species which may have hosted the second and third species.

Note that the first species' polynucleotides S1-1, S1-2, and S1-3 need not necessarily be completely degraded in order to render these polynucleotides unavailable for amplification and sequencing. For example, amplification adapters 180 may be configured to as to selectively become ligated to any double-stranded polynucleotides, and so as substantially not become ligated to any single-stranded polynucleotides. As such, any double-stranded polynucleotides in the mixture to which amplification adapters were ligated may be amplified and then sequenced, whereas any single-stranded polynucleotides may not be amplified because they lack suitable amplification adapters. Illustratively, tagmentation may add adaptors only to dsDNA and may not add adaptors to ssDNA. As another example, T4 DNA ligase may work only on dsDNA. In this regard, note that amplification adaptors 180 may be blunt or A tailed in either such approach.

FIG. 1K illustrates an example flow of operations in a method for treating a mixture of first double-stranded polynucleotides from a first species and second double-stranded polynucleotides from a second species. Method 1000 illustrated in FIG. 1K may include, in the mixture, protecting ends of the first double-stranded polynucleotides and any ends of the second double-stranded polynucleotides (operation 1001). For example, in a manner such as described with reference to FIG. 1B, protective elements 150 may be added to ends of first double-stranded polynucleotides S1-1, S1-2, and S1-3 and of second double-stranded polynucleotide S2-1, while double-stranded polynucleotide S3-1 lacks ends and as such may not become coupled to protective elements 150.

Method 1000 illustrated in FIG. 1K also may include, after protecting the ends of the first and second double-stranded polynucleotides, selectively generating free ends within the first double-stranded polynucleotides (operation 1002). For example, in a manner such as described with reference to FIG. 1C, Cas-gRNA RNPs 160 may be selectively hybridized with sequences that are within the first species' polynucleotides S1-1, S1-2, and S1-3 and that are not within the second species' polynucleotide S2-1 (or third species' polynucleotide S3-1), such as species specific repetitive elements. The Cas-gRNA RNPs 160 may cut the first species' polynucleotides S1-1, S1-2, and S1-3 to generate free ends such as described with reference to FIG. 1D. Method 1000 illustrated in FIG. 1K also may include degrading the first double-stranded polynucleotides from the free ends toward the protected ends (operation 1003). For example, in a manner such as described with reference to FIGS. 1E-1G, exonucleases may be used to degrade the first species' polynucleotides S1-1, S1-2, and S1-3 from the respective free ends 141, 141′, 142, 142′, and 143, 143′. Amplification adapters subsequently may be coupled to the second species' polynucleotide S2-1 in a manner such as described with reference to FIGS. 1I-1J (optionally including fragmentation before adding the amplification adapters), and the polynucleotide then amplified and sequenced.

Accordingly, as provided herein, Cas-gRNA RNPs may be used to selectively generate free ends in the polynucleotides of a desired species, and those polynucleotides subsequently degraded in such a manner as to substantially render them unavailable for amplification or sequencing, in favor of the polynucleotides of one or more other species which may be amplified and sequenced.

Fragmentation of Whole Genome (WG) into Different, Defined Fragment Sizes

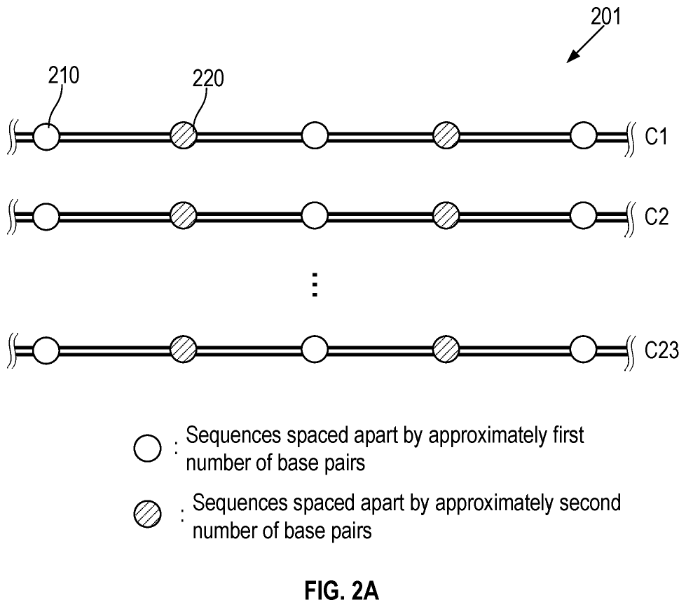

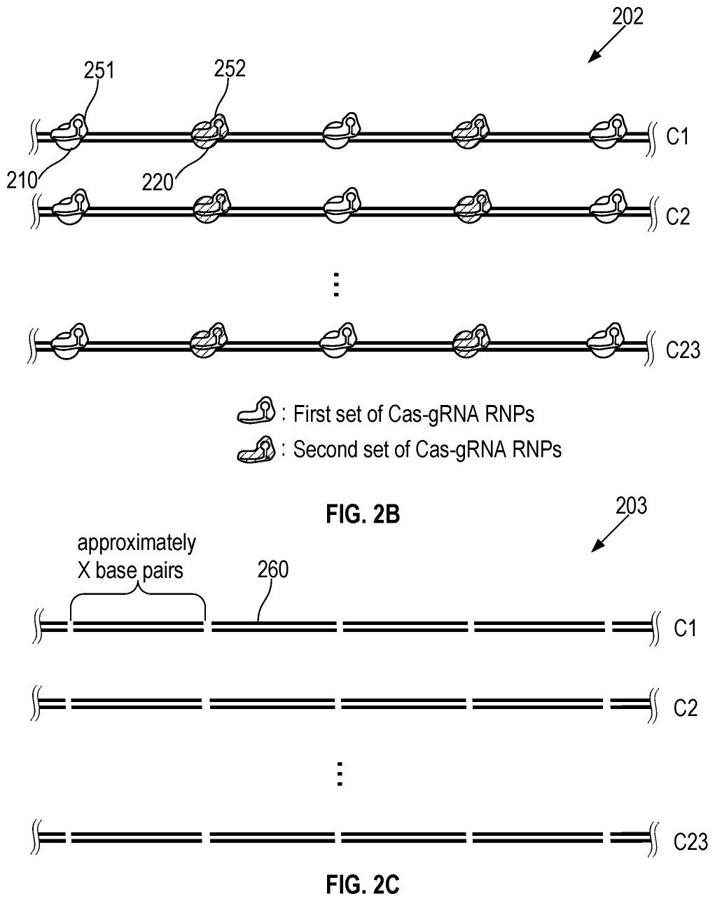

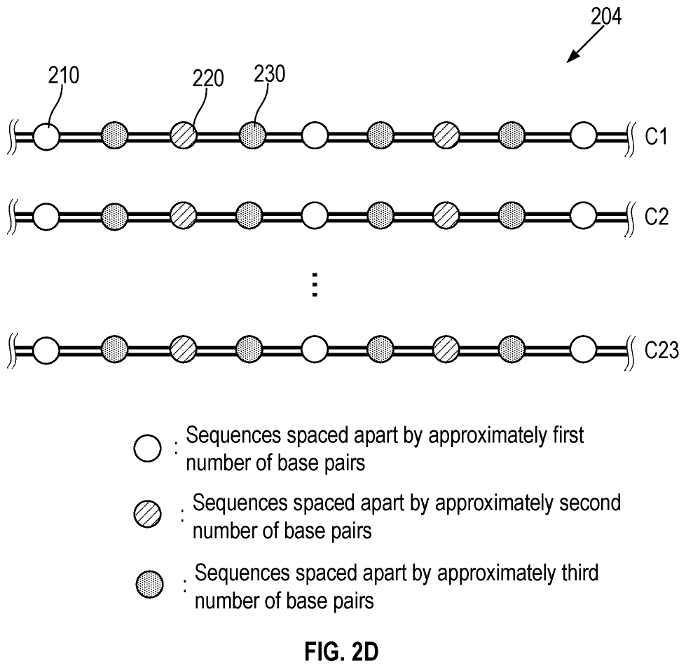

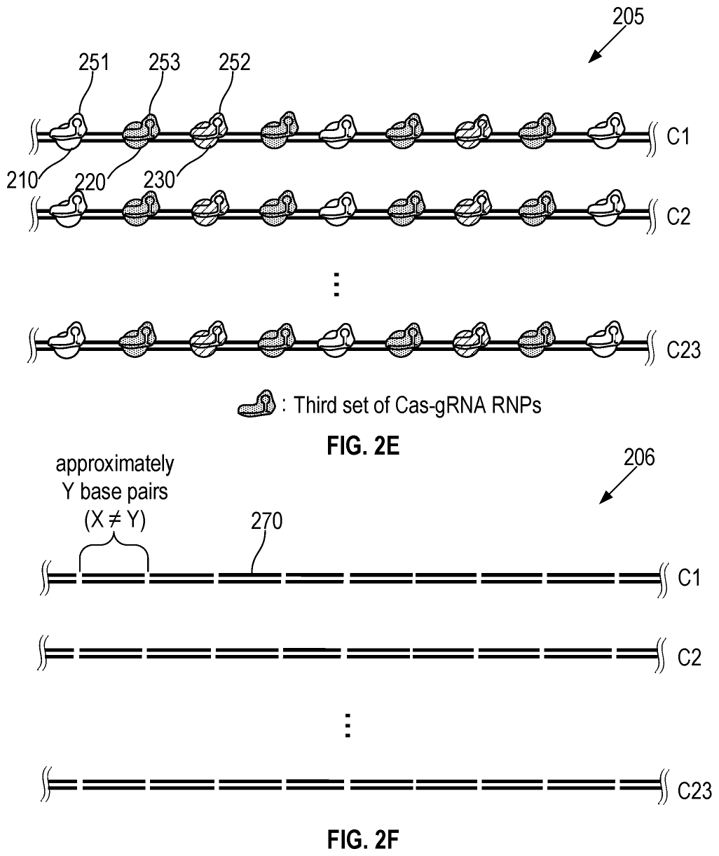

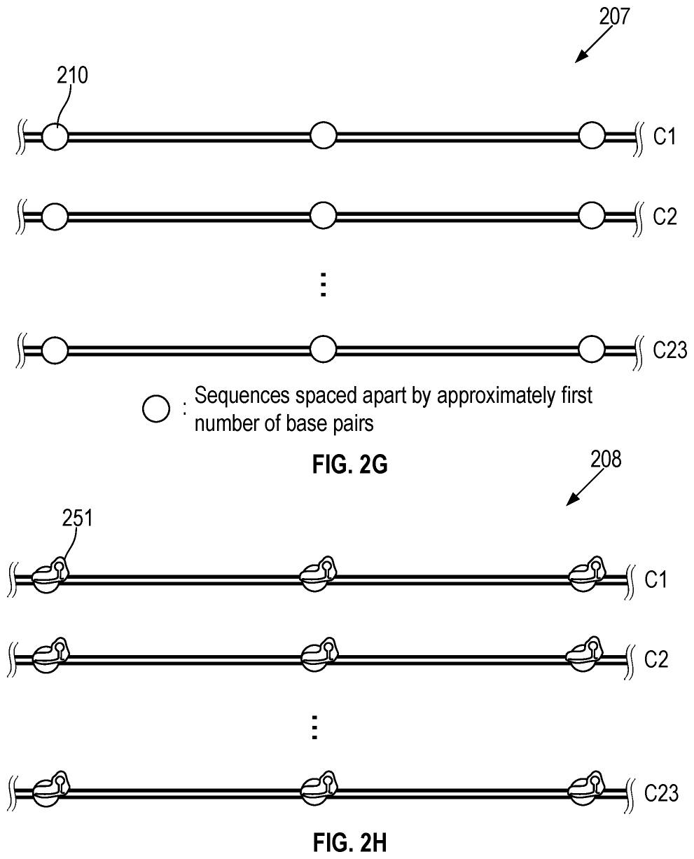

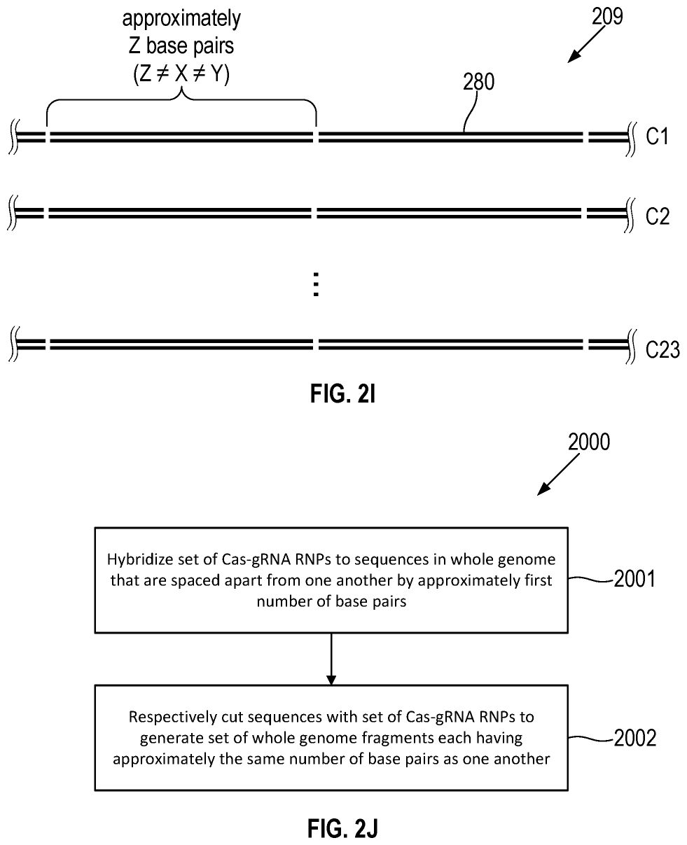

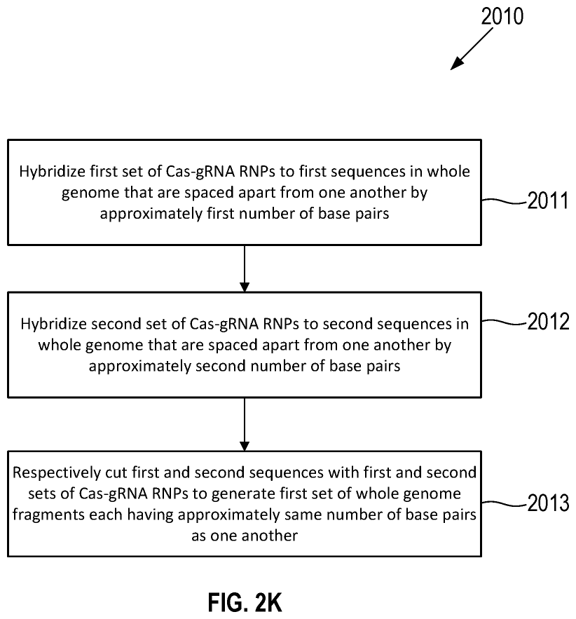

Some examples herein relate to fragmentation of a whole genome (WG) into different, defined fragment sizes. For example, FIGS. 2A-2K schematically illustrate example compositions and operations in a process flow for fragmenting a WG into different, defined fragment sizes.

Depending on the species, the WG of that species includes a well-defined number of chromosomes. The general sequence of each of the human chromosomes has been well characterized, although the sequence of each individual's chromosome includes genetic variations that are specific to that individual. Additionally, the sequence for one or more chromosomes sometimes may vary even within an individual, for example if the individual has a tumor with a different genetic variation than does that individual's normal tissue; a tumor even may have different genetic variations at different locations. These and other types of genetic variations make it desirable to perform WG sequencing. Typically, WG sequencing begins by obtaining an aliquot of blood or other fluid or tissue from an individual, purifying the DNA within that aliquot, and then fragmenting that DNA into smaller fragments that are of a suitable size to be sequenced. Depending on the particular instrument being used to sequence the DNA, it may be that only fragments of a certain size range (e.g., about 100 to about 1000 base pairs) suitably may be sequenced. However, previously known methods of fragmenting DNA using mechanical processes, such as sonication or enzymatic fragmentation, generate a relatively wide distribution of different fragment sizes. Only a small portion of the fragments within that distribution (e.g., about 20%) may have a size in the range that is suitable for sequencing, and the remaining portion of the WG (e.g., about 80%) may be discarded. As provided herein, a WG—or any other suitable polynucleotide or collection of polynucleotides—may be fragmented into any desired number of different fragment sizes, each of which fragment sizes may be relatively well controlled.

For example, as illustrated in FIG. 2A, a first purified sample 201 of the WG may be obtained that includes some, or even all, of the chromosomes of a given species. In the nonlimiting example illustrated in FIG. 2A, sample 201 includes the WG of a human, and as such includes twenty-three DNA chromosomes C1, C2, . . . C23. It will be appreciated that a given sample that may be processed such as provided herein may include any suitable number of any suitable type of polynucleotides. The chromosomes C1, C2, . . . C23 within sample 201 include different sequences 210, 220 along their length, and different portions of those sequences may be used as predefined targets for Cas-gRNA RNPs to be used to cut the chromosomes at approximately evenly spaced locations so as to form approximately evenly sized fragments. Illustratively, first sequences 210 may be spaced apart from one another by approximately a first number of base pairs, and second sequences 220 may be spaced apart from one another by approximately a second number of base pairs. Note that sequences 210 need not include the same particular sequence at each individual location, and similarly sequences 220 need not include the same particular sequence at each individual location. Instead, sequences 210 represent a first set of selected locations within the different chromosomes that are used as predefined targets for a first set of Cas-gRNA RNPs, each of which RNPs may be targeted to a specific one of sequences 210, and sequences 220 represent a second set of selected locations within the different chromosomes that are used as predefined targets for a second set of Cas-gRNA RNPs, each of which RNPs may be targeted to a specific one of sequences 220.

Composition 202 illustrated in FIG. 2B includes first set 251 of Cas-gRNA RNPs hybridized to first sequences 210, and second set 252 of Cas-gRNA RNPs 252 hybridized to second sequences 220. The first set 251 and second set 252 of Cas-gRNA RNPs respectively may be for cutting the first and second sequences within the sample to generate WG fragments each having approximately the same number of base pairs as one another. The Cas may include Cas9. The first set 251 and second set 252 of Cas-gRNA RNPs each may include any suitable number of Cas-gRNA RNPs. Each given one of the RNPs of the first set 251 may be the same as one or more other RNPs in the first set or in the second set, in which case such RNPs may target the same specific sequence 210 or 220 as each other, or may be different than a plurality of other RNPs in the first set or in the second set, in which case that RNP targets a different specific sequence than such other RNPs. Similarly, each given one of the RNPs of the second set 252 may be the same as one or more other RNPs in the first set or in the second set, in which case such RNPs may target the same specific sequence 210 or 220 as each other, or may be different than a plurality of other RNPs in the first set or the second set, in which case that RNP targets a different specific sequence than such other RNPs.

The number of RNPs in each of the first and second sets 251, 252 of Cas-gRNA RNPs suitably may be selected so as to fragment a desired polynucleotide (e.g., one or more double-stranded DNA chromosomes, or an entire set of double-stranded DNA chromosomes). Illustratively, the first set 251 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs. Illustratively, the second set 252 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs.

Composition 203 illustrated in FIG. 2C results from such cutting by first set 251 and second set 252 of Cas-gRNA RNPs, and includes, or consists essentially of, a set of fragments 260 each including approximately X base pairs. As such, substantially the entire WG (or any suitable polynucleotide(s)) in first sample 201 may be fragmented into fragments 260 of defined size. It will be appreciated that the particular locations of sequences 210, 220 along chromosomes C1, C2, . . . C23 that respectively are targeted by the first and second sets 251, 252 of Cas-gRNA RNPs may be selected so as to provide any suitable length of fragments 260. In this particular example, the first number of base pairs by which sequences 210 are spaced apart is approximately the same as the second number of base pairs by which sequences 220, such that sequences 210 and 220 substantially alternate along the length of each chromosome. Illustratively, the first number of base pairs may be between about 100 and about 2000 (e.g., between about 500 and about 700), and the second number of base pairs may be between about 100 and about 2000 (e.g., between about 500 and about 700), or the first number of base pairs may be between about 1000 base pairs and about 3000 base pairs (illustratively, about 2000 base pairs), and the first number of base pairs may be between about 1000 base pairs and about 3000 base pairs (illustratively, about 2000 base pairs).

Because sequences 210 and 220 collectively are at suitably predefined and relatively evenly spaced locations, the number of base pairs in each of fragments 260 may have a relatively tight distribution. For example, the number of base pairs in WG fragments 260 may vary by less than about 20%, or less than about 10%, or less than about 5%, or less than about 2%, or even less than about 1%. The number of base pairs (X) in each of WG fragments 260 may be, illustratively, between about 100 base pairs and about 1000 base pairs, for example between about 200 base pairs and about 400 base pairs (e.g., about 300 base pairs), or may be between about 1000 base pairs and about 3000 base pairs (illustratively, about 2000 base pairs).

Note that the first and/or second sets of Cas-gRNA RNPs may be used to generate WG fragments having other lengths. Indeed, for a given WG, it may be desirable to generate fragments having different, defined lengths than one another and then to compare the sequences that are obtained using each of such different, defined lengths. As provided herein, different fragment lengths respectively may be generated within different samples of the WG (or different samples of other polynucleotides). For example, as illustrated in FIG. 2D, a second purified sample 204 of the WG may be obtained that, like sample 201 illustrated in FIG. 2A, includes twenty-three DNA chromosomes C1, C2, . . . C23 having first sequences 210 spaced apart from one another by approximately a first number of base pairs, and second sequences 220 spaced apart from one another by approximately a second number of base pairs. Although not specifically illustrated in FIG. 2A, chromosomes C1, C2, . . . C23 may include other sequences that may represent other sets of selected locations within the different chromosomes that may be used as predefined targets for a first set of Cas-gRNA RNPs. For example, sequences 230 illustrated in FIG. 2D represent a third set of selected locations within the different chromosomes that are used as predefined targets for a third set of Cas-gRNA RNPs, each of which RNPs may be targeted to a specific one of sequences 230.

Composition 205 illustrated in FIG. 2E includes first set 251 of Cas-gRNA RNPs hybridized to first sequences 210 and second set 252 of Cas-gRNA RNPs 252 hybridized to second sequences 220, as well as third set 253 of Cas-gRNA RNPs hybridized to third sequences 230. In a manner similar to that described with reference to FIG. 2B, the first set 251, second set 252, and third set 253 of Cas-gRNA RNPs respectively may be for cutting the first, second, and third sequences within the sample to generate WG fragments each having approximately the same number of base pairs as one another. The Cas may include Cas9. In a manner similar to that described with reference to FIG. 2B, the first set 251, second set 252, and third set 253 of Cas-gRNA RNPs each may include any suitable number of Cas-gRNA RNPs. Each given one of the RNPs of the first set 251 may be the same as one or more other RNPs in the first set, second set, or third sets, in which case such RNPs may target the same specific sequence 210, 220, or 230 as each other, or may be different than a plurality of other RNPs in the first set, second set, or third set, in which case that RNP targets a different specific sequence than such other RNPs. Similarly, each given one of the RNPs of the second set 252 may be the same as one or more other RNPs in the first set, second set, or third set, in which case such RNPs may target the same specific sequence 210, 220, or 230 as each other, or may be different than a plurality of other RNPs in the first set, second set, or third set, in which case that RNP targets a different specific sequence than such other RNPs. Similarly, each given one of the RNPs of the third set 253 may be the same as one or more other RNPs in the first set, second set, or third set, in which case such RNPs may target the same specific sequence 210, 220, or 230 as each other, or may be different than a plurality of other RNPs in the first set, second set, or third set, in which case that RNP targets a different specific sequence than such other RNPs.

The number of RNPs in each of the first, second, and third sets 251, 252, 253 of Cas-gRNA RNPs suitably may be selected so as to fragment a desired polynucleotide (e.g., one or more double-stranded DNA chromosomes, or an entire set of double-stranded DNA chromosomes). Illustratively, the first set 251 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs. Illustratively, the second set 252 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs. Illustratively, the third set 253 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs.

Composition 206 illustrated in FIG. 2F results from such cutting by first set 251, second set 252, and third set 253 of Cas-gRNA RNPs, and includes, or consists essentially of, a set of fragments 270 each including approximately Y base pairs (X≠Y). As such, substantially the entire WG (or any suitable polynucleotide(s)) in second sample 204 may be fragmented into fragments 270 of defined size. It will be appreciated that the particular locations of sequences 210, 220, 230 along chromosomes C1, C2, . . . C23 that respectively are targeted by the first, second, and third sets 251, 252, 253 of Cas-gRNA RNPs may be selected so as to provide any suitable length of fragments 270. In this particular example, the first number of base pairs by which sequences 210 are spaced apart is approximately the same as the second number of base pairs by which sequences 220, such that sequences 210 and 220 substantially alternate along the length of each chromosome in a manner similar to that described with reference to FIGS. 2A-2C. However, the third number of base pairs by which sequences 230 are spaced apart may differ from the first and/or second numbers of base pairs. As such, although sequences 210 and 220 may substantially alternate along the length of each chromosome, sequences 230 may be regularly interposed between different ones of sequences 210 and 220 in a manner such as illustrated in FIG. 2E. Illustratively, the first number of base pairs may be between about 100 and about 2000 (e.g., between about 500 and about 700), the second number of base pairs is between about 100 and about 2000 (e.g., between about 500 and about 700), and the third number of base pairs is between about 100 and about 2000 (e.g., between about 200 and about 400), or the first number of base pairs may be between about 1000 and about 3000 (e.g., about 2000), the second number of base pairs may be between about 1000 and about 3000 (e.g., about 2000), and the third number of base pairs number of base pairs may be between about 500 and about 2000 (e.g., about 1000).

Because sequences 210, 220, 230 collectively are at suitably predefined and relatively evenly spaced locations, the number of base pairs in each of fragments 270 may have a relatively tight distribution. For example, the number of base pairs in WG fragments 270 may vary by less than about 20%, or less than about 10%, or less than about 5%, or less than about 2%, or even less than about 1%. The number of base pairs (Y) in each of WG fragments 270 may be, illustratively, between about 100 base pairs and about 1000 base pairs, for example between about 100 base pairs and about 200 base pairs (e.g., about 150 base pairs).

Comparing the processing performed using sample 201 to the processing performed using sample 204, it may be appreciated that the same sets of Cas-gRNA RNPs may be used to generate WG fragments having different lengths than one another. For example, the first and second sets 251, 252 of Cas-gRNA RNPs may be used to generate fragments 260 having length X, and also may be used (in combination with third set 253 of Cas-gRNA RNPs) to generate fragments 270 having length Y (X≠Y). The first, second, and/or third sets of Cas-gRNA RNPs similarly may be used to generate fragments of still other defined lengths for other samples of the WG, without the need to provide still further different sets of Cas-gRNA RNPs.

For example, as illustrated in FIG. 2G, a third purified sample 207 of the WG may be obtained that, like sample 201 illustrated in FIG. 2A and sample 204 illustrated in FIG. 2D, includes twenty-three DNA chromosomes C1, C2, . . . C23 having first sequences 210 spaced apart from one another by approximately a first number of base pairs. Although not specifically illustrated in FIG. 2G, chromosomes C1, C2, . . . C23 may include other sequences that may represent other sets of selected locations within the different chromosomes that may be used as predefined targets for other sets of Cas-gRNA RNPs. For example, sequences 220 illustrated in FIG. 2A and sequences 230 illustrated in FIG. 2D represent other sets of selected locations within the different chromosomes that are used as predefined targets for other sets of Cas-gRNA RNPs. Composition 208 illustrated in FIG. 2H includes first set 251 of Cas-gRNA RNPs hybridized to first sequences 210. In a manner similar to that described with reference to FIG. 2B, the first set 251 of Cas-gRNA RNPs may be for cutting the first sequences 210 within the sample to generate WG fragments each having approximately the same number of base pairs as one another. The Cas may include Cas9. In a manner similar to that described with reference to FIG. 2B, the first set 251 of Cas-gRNA RNPs each may include any suitable number of Cas-gRNA RNPs. Each given one of the RNPs of the first set 251 may be the same as one or more other RNPs in the first set, in which case such RNPs may target the same specific sequence 210 as each other, or may be different than a plurality of other RNPs in the first set, in which case that RNP targets a different specific sequence than such other RNPs. The number of RNPs in the first set 251 of Cas-gRNA RNPs suitably may be selected so as to fragment a desired polynucleotide (e.g., one or more double-stranded DNA chromosomes, or an entire set of double-stranded DNA chromosomes). Illustratively, the first set 251 of Cas-gRNA RNPs may include at least about 50,000 different Cas-gRNA RNPs, or at least about 100,000 different Cas-gRNA RNPs, or at least about 1,000,000 different Cas-gRNA RNPs, or at least about 10,000,000 different Cas-gRNA RNPs, or at least about 20,000,000 different Cas-gRNA RNPs.

Composition 209 illustrated in FIG. 2I results from such cutting by first set 251 of Cas-gRNA RNPs (illustrated in FIG. 2H), and includes, or consists essentially of, a set of fragments 280 each including approximately Z base pairs (X≠Y≠Z). As such, substantially the entire WG (or any suitable polynucleotide(s)) in third sample 207 may be fragmented into fragments 280 of defined size. It will be appreciated that the particular locations of sequences 210 along chromosomes C1, C2, . . . C23 that respectively are targeted by the first set 251 of Cas-gRNA RNPs may be selected so as to provide any suitable length of fragments 280. Illustratively, the first number of base pairs may be between about 100 and about 2000 (e.g., between about 500 and about 700, e.g., about 600, or between about 200 and about 400, e.g., about 300), or may be between about 1000 base pairs and about 3000 base pairs, e.g., about 2000. Because sequences 210 collectively are at suitably predefined and relatively evenly spaced locations, the number of base pairs in each of fragments 280 may have a relatively tight distribution. For example, the number of base pairs in WG fragments 280 may vary by less than about 20%, or less than about 10%, or less than about 5%, or less than about 2%, or even less than about 1%. The number of base pairs (Z) in each of WG fragments 280 may be, illustratively, between about 100 base pairs and about 1000 base pairs, for example between about 500 and about 700 base pairs (e.g., about 600), or between about 200 and about 400 base pairs (e.g., about 300), or may be between about 1000 base pairs and about 3000 base pairs, e.g., about 2000.

It will be appreciated that instead of using first set 251 of Cas-gRNA RNPs with third sample 207, either the second set 252 or third set 253 may be used instead of first set 251, so as instead to target sequences 220 or 230 which may provide fragments having other lengths. It will also be appreciated that any suitable number of samples (including one sample) of any suitable number of polynucleotides (including one polynucleotide) may be prepared using any suitable number of sets of Cas-gRNA RNPs (including one set). For example, FIG. 2J illustrates a flow of operations in a method of generating fragments of a WG. Method 2000 illustrated in FIG. 2J includes hybridizing a set of Cas-gRNA RNPs to sequences in a sample of the WG that are spaced apart from one another by approximately a number of base pairs (operation 2001). The resulting composition may include the set of Cas-gRNA RNPs hybridized to sequences in the sample of the WG that are spaced apart from one another by approximately the number of base pairs. The set of Cas-gRNA RNPs respectively may be for cutting the sequences within the sample to generate WG fragments each having approximately the same number of base pairs as one another. For example, method 2000 illustrated in FIG. 2J may include respectively cutting the sequences with the set of Cas-gRNA RNPs to generate a set of WG fragments each having approximately the same number of base pairs as one another (operation 2002). The number of base pairs between the sequences may be between about 100 and about 2000, e.g., between about 500 and about 700 (e.g., about 600), or between about 200 and about 400 (e.g., about 300), or between about 100 and about 200 (e.g., about 150), or may be between about 1000 base pairs and about 3000 base pairs, e.g., about 2000. In some examples, the number of base pairs in the WG fragments may be between about 100 and about 2000, e.g., between about 100 and about 200 (e.g., about 150), or between about 200 and about 400 (e.g., about 300), or between about 500 and about 700 (e.g., about 600) or may be between about 1000 base pairs and about 3000 base pairs, e.g., about 2000. The number of base pairs in the WG fragments of the set of WG fragments may vary by less than about 20%.

Additionally, or alternatively, in other samples one or more other sets of Cas-gRNA RNPs may be used in combination with each other to generate fragments of a WG. For example, FIG. 2K illustrates a flow of operations in another method of generating fragments of a WG in a sample of the WG. Method 2010 illustrated in FIG. 2K may include hybridizing a first set of Cas-gRNA RNPs to first sequences in the WG that are spaced apart from one another by approximately a first number of base pairs (operation 2011). Method 2010 illustrated in FIG. 2K also may include hybridizing a second set of Cas-gRNA RNPs to second sequences in the WG that are spaced apart from one another by approximately a second number of base pairs (operation 2012). Operations 2011 and 2012 may be performed concurrently with one another, e.g., by contacting the sample of the WG with the first and second sets of Cas-gRNA RNPs. Alternatively, the sample may be contacted with the first set of Cas-gRNA RNPs and subsequently contacted with the second set of Cas-gRNA RNPs, or vice versa. Method 2010 illustrated in FIG. 2K also may include respectively cutting the first and second sequences with the first and second sets of Cas-gRNA RNPs in the first sample to generate a first set of WG fragments each having approximately the same number of base pairs as one another. The first and second sequences may be cut concurrently with one another; alternatively, the first sequences may be cut with the first set of Cas-gRNA RNPs and subsequently cut with the second set of Cas-gRNA RNPs, or vice versa. It will be appreciated that FIG. 2K suitably may be modified so as to use one or more additional sets of Cas-gRNA RNPs to cut additional sequences, e.g., in a manner such as described with reference to FIGS. 2D-2F.

Regardless of the particular number of sets of Cas-gRNA RNPs used to cut the polynucleotide(s) in a given sample, it will be appreciated that the resulting fragments may be amplified and sequenced. For example, amplification adapters may be ligated to the ends of the fragments in a similar manner as described with reference to FIG. 1J, amplicons may be generated of the fragments having the amplification adapters ligated thereto, and the amplicons sequenced. For example, amplification adapters may be ligated to the ends of fragments 260, fragments 270, and/or fragments 280 and such fragments then amplified and sequenced. In some examples, the amplification adapters include unique molecular identifiers (UMIs). The different sets of fragments may be amplified and sequenced separately from one another, or may be mixed together for the amplification and/or sequencing. Illustratively, amplicons of any suitable ones of fragments 260, 270, and/or 280 may be mixed together for amplification and/or sequencing.

Accordingly, a composition is provided herein that includes, or consists essentially of, a set of at least about 1,000,000 WG fragments each having approximately the same number of base pairs as one another. Illustratively, the number of base pairs may be between about 100 and about 200 (e.g., about 150), or between about 200 and about 400 (e.g., about 300), or between about 500 and about 700 (e.g., about 600), or between about 1000 and about 3000, e.g., about 2000. The composition may be derived from the whole genome of a species, and may be amplified and sequenced so as to provide the sequence of the whole genome. The size of WG fragments may be tailored for use with the sequencing technique being used, and substantially the entire WG in a given sample may be sequenced, in comparison to mechanical fragmentation techniques in which a relatively low portion of the WG may be of a length that usable for sequencing.

### Labeling Polynucleotides Using Cuts

As noted elsewhere herein, unique molecular identifiers (UMIs) may be coupled to respective polynucleotides as a way to label those polynucleotides for sequencing. Illustratively, any amplicons of a given polynucleotide molecule coupled to a given UMI may also include that UMI, via which those amplicons may be uniquely identified as being derived from that polynucleotide molecule as compared to from other polynucleotide molecules coupled to other UMIs. However, such UMIs may become mutated during the amplification process, and such mutations may inhibit the ability to identify the polynucleotide molecule from which the amplicons are derived. As provided herein, Cas-gRNA RNPs may be used to cut polynucleotide molecules in such a way as to label those polynucleotide molecules and their amplicons for sequencing, without the need for UMIs although such UMIs optionally may be coupled to polynucleotides that are cut in a manner such as provided herein.

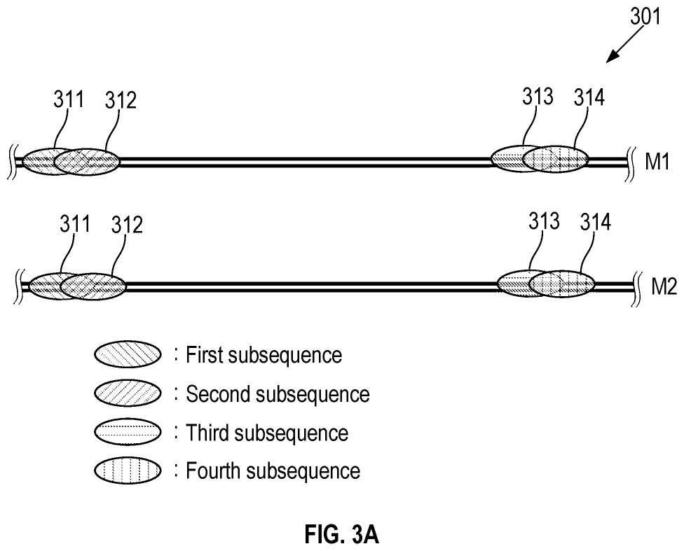

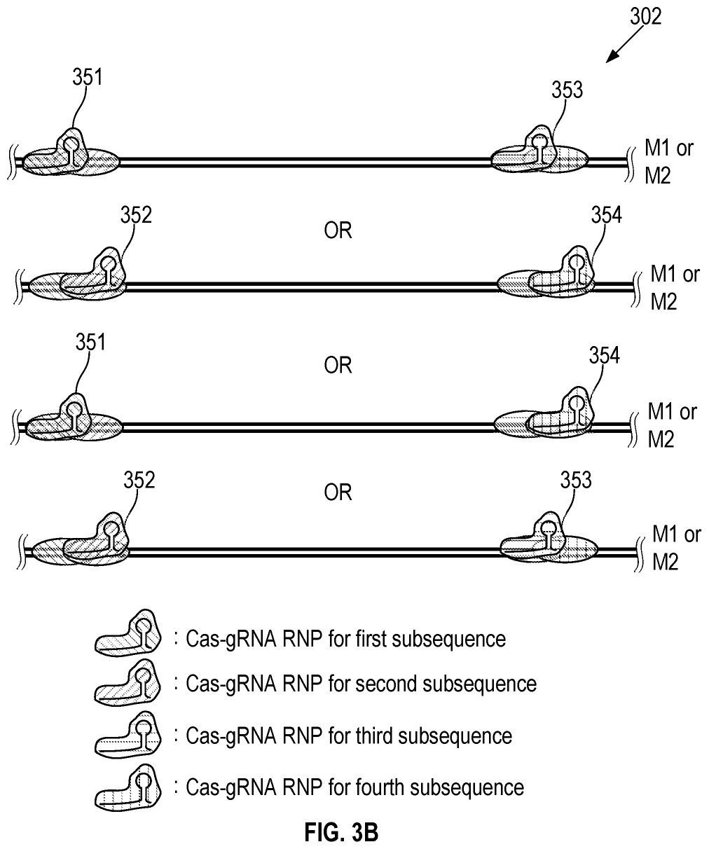

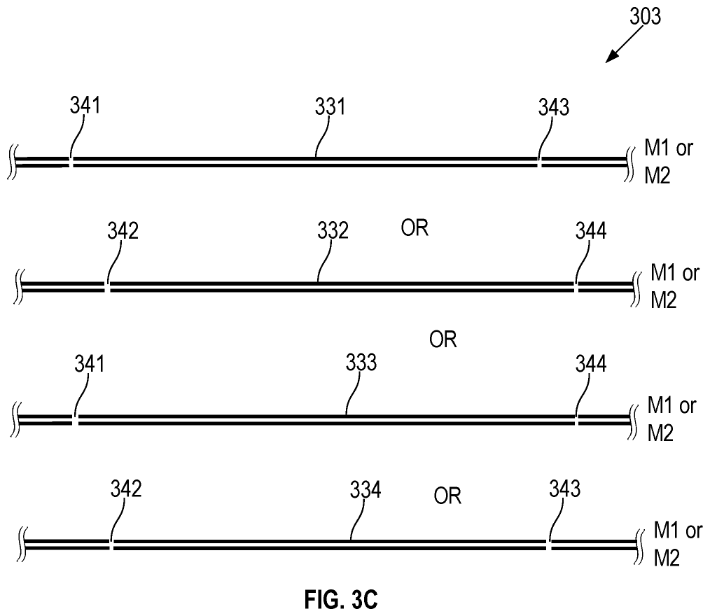

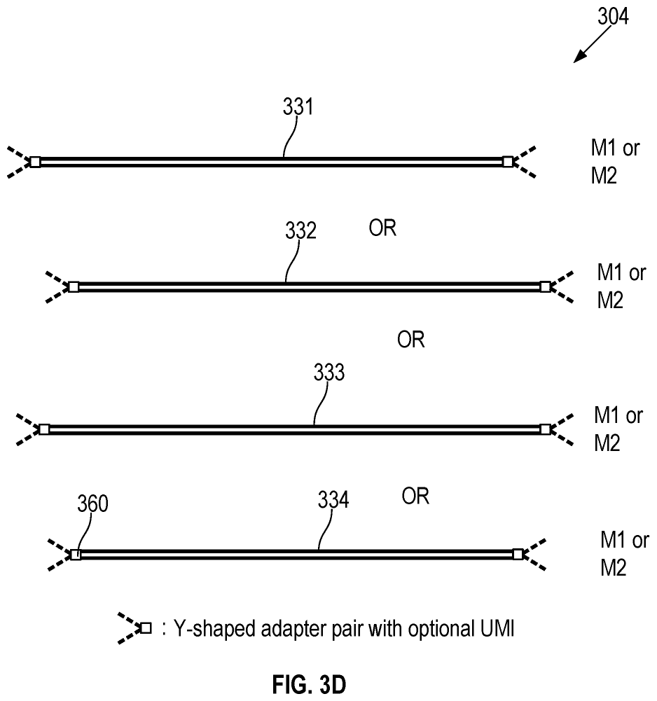

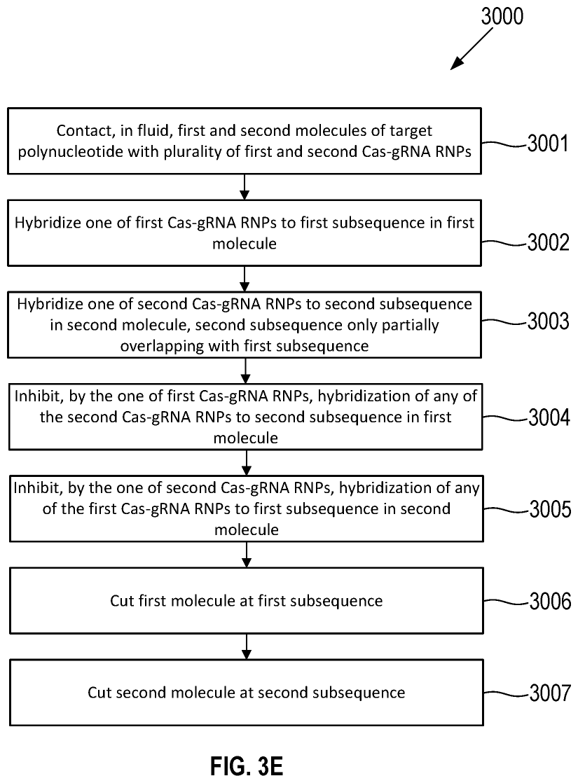

For example, FIGS. 3A-3E schematically illustrate example compositions and operations in a process flow for labeling polynucleotides using cuts. FIG. 3A illustrates composition 301 including first and second molecules M1, M2 of a target polynucleotide, such as double-stranded DNA. Each of the molecules M1, M2 may have substantially the same sequence, and as such which molecule is considered to be “first” and which is “second” is arbitrary. The sequence of the target polynucleotide may include different subsequences that may be used to cut the polynucleotide molecules M1, M2 at one or more different locations than one another, and the respective locations of such cuts may be considered to label the respective polynucleotide molecules. For example, each of the polynucleotide molecules may include first subsequence 311 to which a first Cas-gRNA RNP may be targeted (that is having a sequence that is complementary to a relevant portion the gRNA), second subsequence 312 to which a second Cas-gRNA RNP may be targeted, third subsequence 313 to which a third Cas-gRNA RNP may be targeted, and fourth subsequence 314 to which a fourth Cas-gRNA RNP may be targeted. First and second subsequences 311, 312 may only partially overlap with one another, and third and fourth subsequences 313, 314 may only partially overlap with one another.

In composition 302 illustrated in FIG. 3B, first and second molecules M1, M2 of the target polynucleotide are contacted in a fluid with a plurality of each of the first and second Cas-gRNA RNPs, 351, 352, and also may be contacted with a plurality of each of the third and fourth Cas-gRNA RNPs 353, 354. Depending on which of the RNPs initially hybridize with the corresponding subsequence within each of the molecules M1, M2, others of the RNPs may be inhibited from hybridizing with other subsequences within those molecules, in a manner such as illustrated in FIG. 3B. In one nonlimiting example, one of the first Cas-gRNA RNPs 351 may hybridize to first subsequence 311 in first molecule M1, and one of the second Cas-gRNA RNPs may hybridize to second subsequence 312 in second molecule M2. Because the first and second subsequences 311, 312 only partially overlap with one another, the one of the first Cas-gRNA RNPs 351 that is hybridized to first molecule M1 may inhibit hybridization of any of the second Cas-gRNA RNPs 351 to the second subsequence 312 in first molecule M1, and the one of the second Cas-gRNA RNPs 352 that is hybridized to second molecule M2 may inhibit hybridization of any of the first Cas-gRNA RNPs 351 to the first subsequence 311 in the second molecule M2. That is, once one of the first Cas-gRNA RNPs 351 hybridizes to one of the molecules, the second Cas-gRNA RNPs 352 may not also hybridize to that molecule, and once one of the second Cas-gRNA RNPs 352 hybridizes to one of the molecules, the first Cas-gRNA RNPs 351 may not also hybridize to that molecule. In a manner such as described in greater detail with reference to FIG. 3C, the molecules then may be cut at the first or second subsequence 311, 312 to which the first or second Cas-gRNA RNP 351, 352 is hybridized. As such, the cuts may be at different locations than one another. Illustratively, the cut in first molecule M1 may be at a different location in the sequence of the target polynucleotide than the cut in the second molecule M2. It will be appreciated that in some circumstances the same type of RNP may hybridize to both the first and second molecules M1, M2, in which case the molecules may be cut at the same location.

In a manner such as illustrated in FIG. 3B, third and fourth Cas-gRNA RNPs 353, 354 similarly may hybridize to the third or fourth subsequences 313, 314 and may inhibit hybridization of other RNPs to those subsequences. For example, one of the third Cas-gRNA RNPs 353 may hybridize to third subsequence 313 in first molecule M1, and may inhibit hybridization of any of the fourth Cas-gRNA RNPs to fourth subsequence 314 in first molecule M1. In a manner such as described in greater detail with reference to FIG. 3C, the first molecule M1 then may be cut at the third subsequence using the one of the third Cas-gRNA RNPs 353 to generate a fragment. Alternatively, one of the fourth Cas-gRNA RNPs 354 may hybridize to fourth subsequence 354 in first molecule M1 and may inhibit hybridization of any of the third Cas-gRNA RNPs to the third subsequence in the first molecule. In a manner such as described in greater detail with reference to FIG. 3C, the first molecule M1 then may be cut at the fourth subsequence using the one of the fourth Cas-gRNA RNPs 354 to generate a fragment. The RNPs may hybridize to different subsequences the second molecule M2 in similar fashion. For example, one of the third Cas-gRNA RNPs 353 may hybridize to third subsequence 313 in second molecule M2 and may inhibit hybridization of any of the fourth Cas-gRNA RNPs 354 to the fourth subsequence 314 in the second molecule M2. In a manner such as described in greater detail with reference to FIG. 3C, the second molecule M1 then may be cut at the third subsequence 313 using the one of the third Cas-gRNA RNPs 354 to generate a fragment. Alternatively, one of the fourth Cas-gRNA RNPs 354 may hybridize to fourth subsequence 314 in second molecule M2 and may inhibit hybridization of any of the third Cas-gRNA RNPs 353 to third subsequence 313 in second molecule M2. In a manner such as described in greater detail with reference to FIG. 3C, the second molecule M2 then may be cut at the fourth subsequence using the one of the fourth Cas-gRNA RNPs 354 to generate a fragment. It will be appreciated that in some circumstances the same type of RNP may hybridize to both the first and second molecules M1, M2, in which case the molecules may be cut at the same location. Statistically, however, it is more likely that at least one of the cuts in the first and second molecules may be at a different location than one another in the sequence of the target polynucleotide.

Turning now to FIG. 3C, the first and second molecules M1, M2 may be cut using the Cas-gRNA RNPs to generate composition 303. Illustratively, first molecule M1 may be cut at location 341 using the one of the first Cas-gRNA RNPs 351 hybridized thereto, and second molecule M2 may be cut at location 342 using the one of the second Cas-gRNA RNPs. Similarly, first molecule M1 may be cut at location 343 or 344 using the one of the third or the fourth Cas-gRNA RNPs 353, 354 hybridized thereto, and second molecule M2 may be cut at location 343 or 344 using the one of the third or the fourth Cas-gRNA RNPs 353, 354 hybridized thereto. However, it should be appreciated that any molecule of the target polynucleotide may be cut at any suitable location, e.g., a location to which a Cas-gRNA RNP may hybridize. A cut at location 341 in one molecule may, for example, be offset from cut at location 342 in another molecule by between about two base pairs and about forty base pairs (e.g., about 2-20 base pairs, or about 5-10 base pairs) in the sequence of the target polynucleotide. Similarly, a cut at location 343 in one molecule may, for example, be offset from cut at location 344 in another molecule by between about two base pairs and about forty base pairs (e.g., about 2-20 base pairs, or about 5-10 base pairs) in the sequence of the target polynucleotide. As such, as illustrated in FIG. 3C, depending on the particular combination of cuts 341 or 342 and 343 or 344 made in each of the first and second molecules M1, M2, fragments of different lengths, and having different numbers of base pairs, may be formed. For example, fragment 331 may have a length between the location of cut 341 and cut 343; fragment 332 may have a length between the location of cut 342 and cut 344; fragment 333 may have a length between the location of cut 341 and cut 344; and fragment 334 may have a length between the location of cut 342 and cut 343. Note that fragments 331 and 332 may have approximately the same length as each other, but may be shorter than fragment 333 and longer than fragment 334, because of the particular locations of cuts in the various fragments. Each of fragments 331, 332, 333, 334 may have a length between about 100 base pairs and about 1000 base pairs, e.g., between about 500 base pairs and about 700 base pairs (illustratively, about 600 base pairs), or between about 200 base pairs and about 400 base pairs (illustratively, about 300 base pairs), or between about 100 base pairs and about 200 base pairs (illustratively, about 150 base pairs), or between about 1000 and about 3000 base pairs, e.g., about 2000 base pairs.

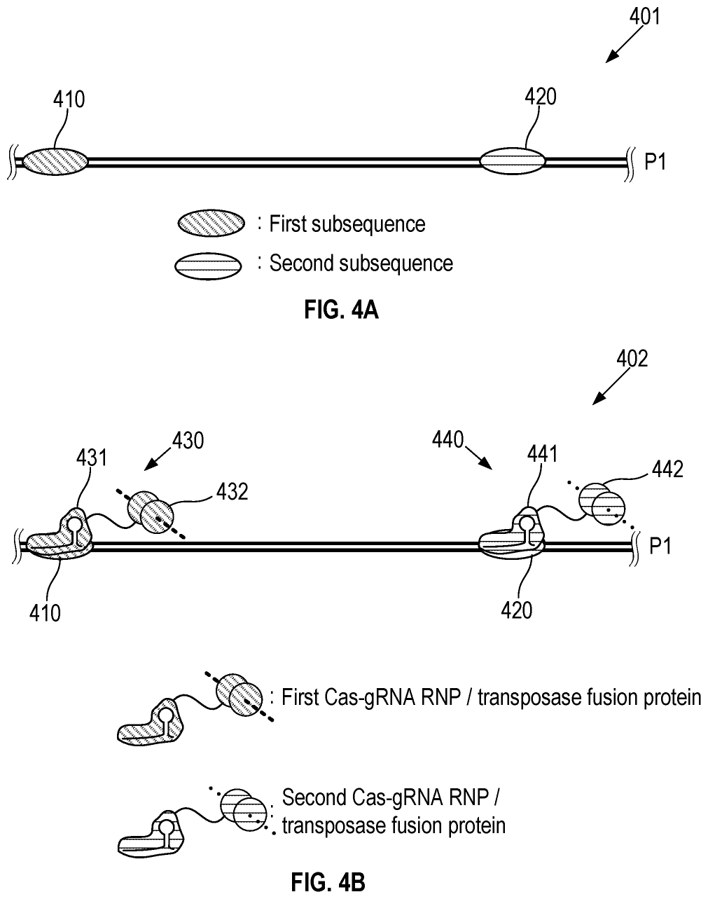

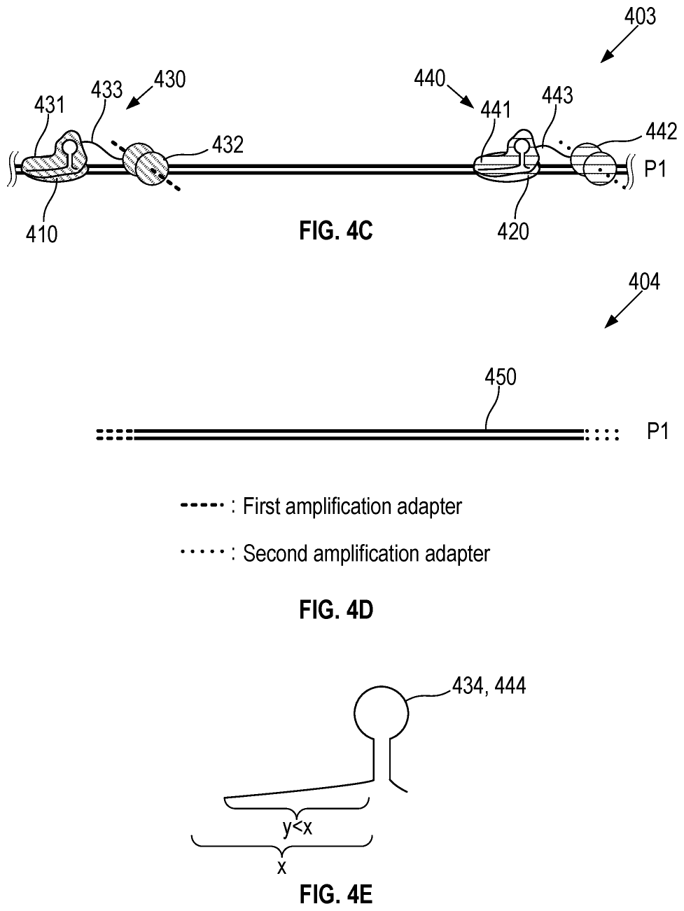

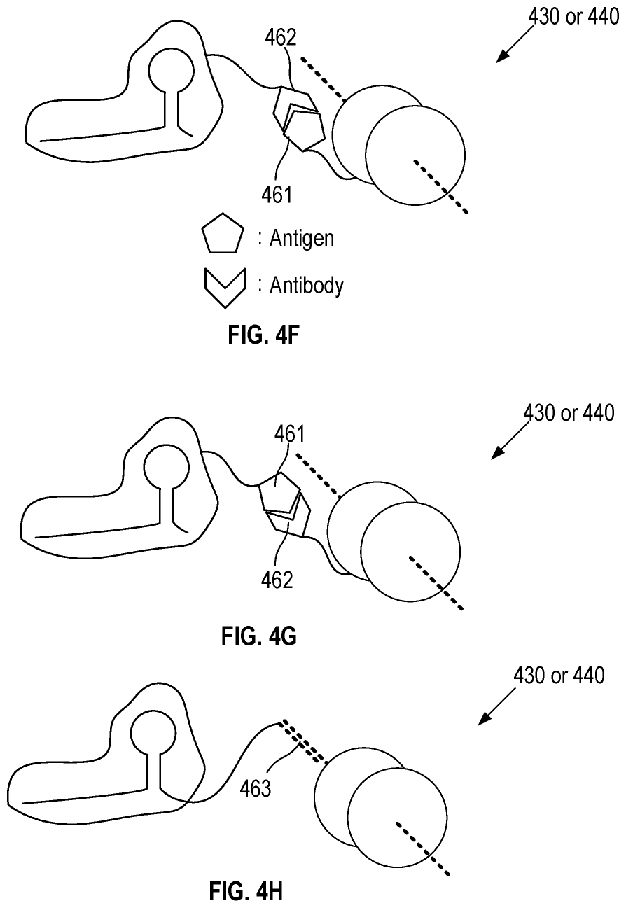

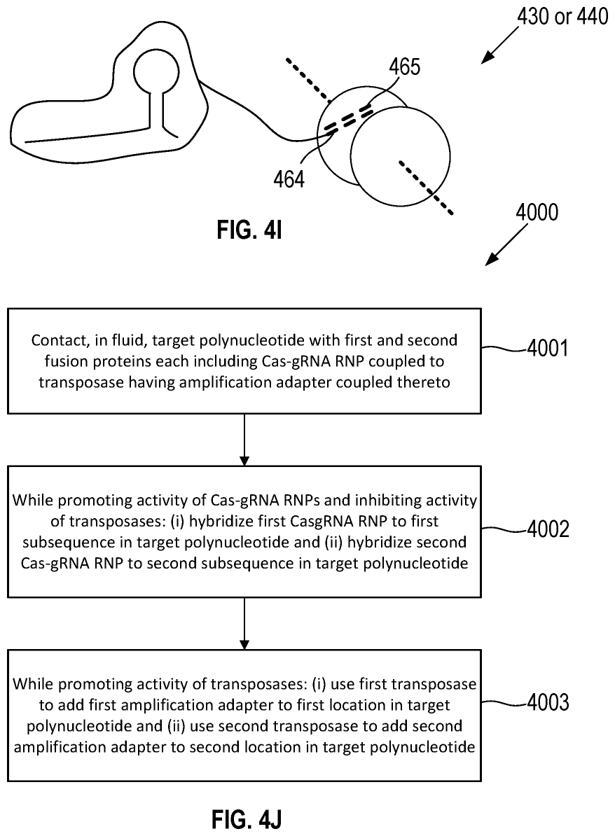

Accordingly, composition 303 illustrated in FIG. 3C may include first and second molecules M1, M2 of a target polynucleotide having a sequence. The first molecule (e.g., fragment 331 or fragment 333) may have a first end at a first subsequence 311, and the second molecule (e.g., fragment 332 or 334) may have a first end at a second subsequence 312. In a manner such as described with reference to FIG. 4, the first subsequence 311, 312 may only partially overlap with the second subsequence. The first end of the first molecule may be at a different location in the sequence of the target polynucleotide than the first end of the second molecule. The first end of the first molecule, may, for example, be offset from the first end of the second molecule by between about two base pairs and about ten base pairs in the sequence of the target polynucleotide. The first molecule (e.g., fragment 331) further may have a second end at a third subsequence 313, and the second molecule (e.g., fragment 332 or 334) further may have a second end at the third subsequence 313 or at a fourth subsequence 314. The third subsequence may only partially overlap with the fourth subsequence. The second end of the first molecule may be at a different location in the sequence of the target polynucleotide than the second end of the second molecule. The second end of the first molecule may be offset from the second end of the second molecule by between about two base pairs and about ten base pairs in the sequence of the target polynucleotide. The first and second molecules may include different numbers of base pairs than one another, or may have the same number of base pairs as one another.

In some examples, the Cas includes Cas9 which cuts the molecule to which the respective Cas-gRNA RNP 351, 352, 353, and/or 354 is hybridized. In other examples, the Cas includes deactivated Cas9 (dCas9). In one nonlimiting example, while one of the first Cas-gRNA RNPs 351 and one of the third or the fourth Cas-gRNA RNPs 353, 354 are hybridized to first molecule M1, any portions of the first molecule that are not between that first Cas-gRNA RNP and that third or fourth Cas-gRNA RNP may be degraded, e.g., using exonuclease III or exonuclease VII. In another nonlimiting example, while one of the second Cas-gRNA RNPs 352 and one of the third or the fourth Cas-gRNA RNPs 353, 354 are hybridized to second molecule M2, any portions of the second molecule that are not between that second Cas-gRNA RNP and that third or the fourth Cas-gRNA RNP may be degraded, e.g., using exonuclease III or exonuclease VII. That is, a suitable exonuclease may be used to degrade portions of a molecule that are not located between Cas-gRNA RNPs hybridize thereto. As such, the Cas-gRNA RNPs may be considered to protect the portion of the molecule therebetween.

Fragments generated using the present methods may be amplified and sequenced. For example, as illustrated in FIG. 3D, amplification adapters 360 may be ligated to the ends of the fragments in a similar manner as described with reference to FIG. 1J, amplicons may be generated of the fragments having the amplification adapters ligated thereto, and the amplicons sequenced. For example, amplification adapters 360 may be ligated to the ends of fragments 331, 332, 333, 334 and such fragments then amplified and sequenced. In some examples, the amplification adapters include unique molecular identifiers (UMIs), however such UMIs are purely optional. Any UMIs may be coupled to, and ligated to the ends of the first and second fragments in the same operation as, the amplification adapters.

First subsequence 311, second subsequence 312, third subsequence 313, and fourth subsequence 314 may be used to identify the amplicons of different fragments as deriving from different ones of the first and second molecules M1, M2. Illustratively, fragment 331 and its amplicons may have a first end at location 341 that falls within subsequence 311 and a second end at location 342 that falls within subsequence 313; fragment 332 and its amplicons may have a first end at location 342 that falls within subsequence 312 and a second end at location 344 that falls within subsequence 314; fragment 333 and its amplicons may have a first end at location 341 that falls within subsequence 311 and a second end at location 344 that falls within subsequence 314; and fragment 334 and its amplicons may have a first end at location 342 that falls within subsequence 312 and a second end at location 332 that falls within subsequence 313. Accordingly, based on the locations of the respective ends of a given amplicon within subsequences 311, 312, 313, 314, it may be determined that such amplicon derived from a particular one of molecules M1 or M2. Any UMIs similarly may be used to identify amplicons as deriving from a particular one of the molecules M1 or M2. This ability to identify all of the reads that derived from a specific molecule allows those reads to be collapsed so as to determine the true sequence of the original molecule. In practice, this may provide error correction and increased accuracy, allowing for identification of true variants as opposed to errors that may have been introduced during preparation and sequencing. This also provides a highly efficient way to add UMIs. In comparison, UMIs that are ligated prior to amplification may suffer from poor conversion efficiencies. The present methods may build in UMI identification into the cutting of the library may be less subject to errors introduced during PCR, and thus more accurate.

FIG. 3E illustrates an example flow of operations in a method for cutting polynucleotides. Method 3000 illustrated in FIG. 3E includes contacting, in a fluid, first and second molecules of the target polynucleotide with a plurality of first and second Cas-gRNA RNPs (operation 3001). Method 3000 illustrated in FIG. 3E includes hybridizing one of the first Cas-gRNA RNPs to a first subsequence in the first molecule (operation 3002). For example, in a manner such as described with reference to FIG. 3B, one of first Cas-gRNA RNPs 351 may hybridize to first subsequence 311 in molecule M1. Method 3000 illustrated in FIG. 3E includes hybridizing one of the second Cas-gRNA RNPs to a second subsequence in the second molecule, the second subsequence only partially overlapping with the first subsequence (operation 3003). For example, in a manner such as described with reference to FIG. 3B, one of second Cas-gRNA RNPs 352 may hybridize to second subsequence 312 in molecule M2. Method 3000 illustrated in FIG. 3E includes inhibiting, by the one of the first Cas-gRNA RNPs, hybridization of any of the second Cas-gRNA RNPs to the second subsequence in the first molecule (operation 3004). For example, a first Cas-gRNA RNP 351 hybridized to molecule M1 may inhibit a second Cas-gRNA RNP 352 from also hybridizing to that molecule. Method 3000 illustrated in FIG. 3E includes inhibiting, by the one of the second Cas-gRNA RNPs, hybridization of any of the first Cas-gRNA RNPs to the first subsequence in the second molecule (operation 3005). For example, a second Cas-gRNA RNP 352 hybridized to molecule M2 may inhibit a first Cas-gRNA RNP 351 from also hybridizing to that molecule. Method 3000 illustrated in FIG. 3E includes cutting the first molecule at the first subsequence (operation 3006), and cutting the second molecule at the second subsequence (operation 3007). Example operations for cutting such molecules using Cas-gRNA RNPs are provided with reference to FIG. 3C.

Accordingly, it may be understood that different molecules of a target polynucleotide may be cut at defined locations so as to generate ends at various locations, and following amplification and sequencing the locations of such ends in the sequence of the target polynucleotide may be used to identify the molecules from which amplicons are derived.

### Coupling Amplification Adapters to Polynucleotides

Coupling amplification adapters to polynucleotides facilitates their amplification and sequencing. As provided herein, amplification adapters may be coupled to polynucleotides using fusion proteins that include both a Cas-gRNA RNP and a transposase. For example, FIGS. 4A-4J schematically illustrate example compositions and operations in a process flow for incorporating amplification adapters into polynucleotides. As illustrated in FIG. 4A, composition 401 may include a target polynucleotide P1 (such as double-stranded DNA) including a first subsequence 410 that may be targeted using a first Cas-gRNA RNP (that is, include a sequence to which the gRNA of the Cas-gRNA RNP may hybridize). Optionally, composition 401 further may include a second subsequence 420 that may be targeted using a second Cas-gRNA RNP. As illustrated in FIG. 4B, target polynucleotide P1 may be contacted with first fusion protein 430, and optional second fusion protein 440 in a fluid. The first fusion protein 430 (and, if present, second fusion protein 440) may be in an approximately stoichiometric ratio to the target polynucleotide P1 in the fluid.

First fusion protein 430 may include first Cas-gRNA RNP 431 coupled to first transposase 432 having a first amplification adapter (indicated by dashed line) coupled thereto. Optional second fusion protein 440 may include second Cas-gRNA RNP 441 coupled to second transposase 442 having a second amplification adapter (indicated by dotted line) coupled thereto. Non-limiting examples for coupling Cas-gRNA RNPs to transposases are provided further below with reference to FIGS. 4F-4I. It will be appreciated that any suitable amplification adapters may be coupled to target polynucleotide using transposases 432, 442. Illustratively, the first amplification adapter may include a P5 adapter, and the second amplification adapter may include a P7 adapter. Optionally, the first amplification adapter also may include a first unique molecular identifier (UMI), and the second amplification adapter may include a second UMI. The UMIs may be used during sequencing in a manner such as described elsewhere herein.

While promoting activity of first Cas-gRNA RNP 431 (and, if present, second Cas-gRNA RNP 441) and inhibiting activity of first transposase 432 (and, if present, second transposase 442), composition 402 illustrated in FIG. 4B may be provided in which first Cas-gRNA RNP 431 is hybridized to first subsequence 410 in target polynucleotide P1, and, if present, second Cas-gRNA RNP 441 is hybridized to second subsequence 420 in the target polynucleotide. In some examples, activity of first and second Cas-gRNA RNPs 431, 441 may be promoted and the activity of transposases 432, 442 may be inhibited using a condition of the fluid. For example, it is well known that different enzymes may use certain ions to function. Illustratively, Cas-gRNA RNPs 431, 441 may use calcium ions (Ca2+), manganese ions (Mn2+) or both calcium ions and manganese ions to function, e.g., respectively to hybridize to sequences 420, 430. In comparison, transposases 432, 442 may use magnesium ions (Mg2+) to function, e.g., to couple amplification adapters to target polynucleotide P. Accordingly, by contacting target polynucleotide P1 with first and second fusion proteins 430, 440 in a fluid having a condition including presence of a sufficient amount of calcium ions, manganese ions, or both calcium and manganese ions for activity of Cas-gRNA RNPs 431, 441 and absence of a sufficient amount of magnesium ions for activity of transposases 432, 442, the Cas-gRNA RNPs may function properly while the transposases may not. Additionally, or alternatively, the binding of the transposase to the target polynucleotide may be inhibited in any suitable manner, e.g., reversibly blocking the binding site on the transposase, using a different temperature to hybridize the Cas-gRNA RNPs than is used to for the transposase, and/or delaying binding of the transposase adaptors to the transposase until after the Cas-gRNA has hybridized to the target polynucleotide so as to delay the transposase's ability to bind to the target polynucleotide, and the like. Optionally, while the Cas-gRNA RNP 431 of first fusion protein 430 is hybridized to first subsequence 410 and the Cas-gRNA RNP 441 of second fusion protein 440 is hybridized to second subsequence 420, any portions of target polynucleotide P1 that are not between the Cas-gRNA RNPs 431, 441 may be degraded, e.g., using exonuclease III or exonuclease VII.

Subsequently, while promoting activity of first and second transposases 432, 442, the first transposase may be used to add the first amplification adapter to a first location in the target polynucleotide P1, and the second transposase may be used to add the second amplification adapter to a second location in the target polynucleotide. For example, activity of transposases 432, 442 may be promoted using a second condition of the fluid, such as presence of a sufficient amount of magnesium ions for activity of the transposases. Illustratively, the magnesium ions may be mixed into the fluid. As such, composition 403 illustrated in FIG. 4C may be provided in which transposases 432, 442 act upon target polynucleotide P1 to couple the first and second amplification adapters thereto. Target polynucleotide P1 may be released from first and second fusion proteins 430, 440 to provide composition 404 illustrated in FIG. 4D which includes fragment 450 of the target polynucleotide P1 having the first amplification adapter at one end, and the second amplification adapter at the other end. Such releasing may be performed using proteinase K, sodium dodecyl sulfate (SDS), or both proteinase K and SDS. Fragment 450 having amplification adapters coupled thereto may be amplified and sequenced.

The length of fragment 450 may be closely related to, e.g., approximately the distance between, first sequence 410 and second sequence 420. For example, as illustrated in FIG. 4C, first Cas-gRNA RNP 431 of fusion protein 430 may be coupled to first transposase 432 via linker 433, and second Cas-gRNA RNP 441 of fusion protein 440 may be coupled to second transposase 442 via linker 443. Nonlimiting examples of linkers 433, 443 are provided in greater detail below with reference to FIGS. 4F-4I. The linkers 433, 443 may have a well defined length and thus may provide a defined distance which the transposases may move from the respective Cas-gRNA RNPs. As such, when the Cas-gRNA RNPs 431, 441 are hybridized to their respective sequences 410, 420 in the target polynucleotide P1 and the transposases 432, 442 are activated (e.g., using a condition of the fluid), the transposases respectively may become coupled to regions of the target polynucleotide that are relatively close to the Cas-gRNA RNPs, in any location that may permitted by the length of linkers 433, 443. However, because the transposases may not couple to specific sequences in the target polynucleotide P1 (as do the Cas-gRNA RNPs), there may be a range of locations to which the transposases respectively may couple. Illustratively, the first location to which transposase 432 adds the first adapter may be within about 10 bases of first subsequence 410, and the second location to which transposase 442 adds the second adapter may be within about 10 bases of second subsequence 420.

It will be appreciated that fragment 450 illustrated in FIG. 4D may have any suitable length, e.g., as approximately defined by the distance between sequences 410, 420 (shown in FIGS. 4A-4C). For example, fragment 450 may have a length of between about 100 base pairs and about 1000 base pairs, e.g., between about 500 base pairs and about 700 base pairs (illustratively, about 600 base pairs), or between about 200 base pairs and about 400 base pairs (illustratively, about 300 base pairs), or between about 100 base pairs and about 200 base pairs (illustratively, about 150 base pairs), or a length of between about 1000 base pairs and about 3000 base pairs (illustratively, about 2000 base pairs).

As illustrated in FIG. 4E, the gRNA 434, 444 respectively within first and second fusion proteins 430, 440 may have any suitable length and sequence to promote its hybridization to respective sequences 410, 420. For example, the 5′ end of gRNA 434, 444 that hybridizes to the first or second subsequence 410, 420 may be truncated relative to that of the gRNA that is more typically used in Cas-gRNA RNPs. Illustratively, as shown in FIG. 4E, typical gRNA may have a 5′ end of length x, where x may be about 20 nucleotides, while gRNA 434, 444 may have a 5′ end of length y, where y is less than x. In some examples, the portion y of the gRNA 434 that hybridizes to first subsequence 410 may have a length of about 15 to about 18 nucleotides, and the portion y of the gRNA 444 that hybridizes to second subsequence 420 may have a length of about 15 to about 18 nucleotides. For further details regarding truncating gRNA, see Fu et al., “Improving CRISPR-Cas nuclease specificity using truncated guide RNAs,” Nat. Biotechnol. 32(3): 279-284 (2014), the entire contents of which are incorporated by reference herein.

It will be appreciated that any suitable Cas and any suitable transposase may be used in fusion proteins 430, 440. Illustratively, the Cas may include dCas9 (e.g., so as to inhibit the Cas from cutting target polynucleotide P1 before the transposase is activated), and the transposase may include Tn5 (e.g., so that the activity of the transposase may be well controlled through fluidic conditions, such as adding as sufficient amount of magnesium ions). The Cas and transposase may be coupled to one another via any suitable linkage, e.g., via a covalent linkage or via a non-covalent linkage. Covalent linkages may be formed, illustratively, copper(I)-catalyzed click reaction, or strain-promoted azide-alkyne cycloaddition. Non-covalent linkages may be formed in any suitable manner. For example, in a manner such as illustrated in FIG. 4F, a Cas-gRNA RNP may be covalently coupled to an antibody 461 and the transposase may be covalently coupled to an antigen 462 to which the antibody is non-covalently coupled, or in a manner such as illustrated in FIG. 4G, the Cas-gRNA RNP may be covalently coupled to an antigen 461 and the transposase may be covalently coupled to an antibody 462 to which the antigen is non-covalently coupled. Alternatively, in a manner such as illustrated in FIG. 4H, the Cas-gRNA may be non-covalently coupled to the transposase via hybridization between a portion 463 of the gRNA and the first or second amplification adapter. As yet another example, in a manner such as illustrated in FIG. 4I, the Cas-gRNA may be non-covalently coupled to the transposase via hybridization between a portion 464 the gRNA and an oligonucleotide 465 within the transposase. For additional examples of a manner in which a Cas may be coupled to another protein, see the following references, the entire contents of which are incorporated by reference herein: Guilinger et al., “Fusion of catalytically inactive Cas9 to Fokl nuclease improves the specificity of genome modification,” Nature Biotechnology 32: 577-582 (2014); and Bhatt et al., “Targeted DNA transposition in vitro using a dCas9-transposase fusion protein,” Nucleic Acids Res. 47: 8126-8135 (2019).

FIG. 4J illustrates an example flow of operations in a method of generating a fragment of a target polynucleotide having a sequence. Method 4000 illustrated in FIG. 4J includes contacting, in a fluid, the target polynucleotide with first and second fusion proteins each including a Cas-gRNA RNP coupled to a transposase having an amplification adapter coupled thereto (operation 4001). For example, target polynucleotide P1 may be contacted with first and second fusion proteins 430, 440 in a manner such as described with reference to FIG. 4B. Method 4000 illustrated in FIG. 4J includes, while promoting activity of the Cas-gRNA RNPs and inhibiting activity of the transposases: (i) hybridizing the first Cas-gRNA RNP to a first subsequence in the target polynucleotide, and (ii) hybridizing the second Cas-gRNA RNP to a second subsequence in the target polynucleotide (operation 4002). For example, the fluid may have a first condition that promotes such hybridizations of first Cas-gRNA RNP 431 to first subsequence 410 and second Cas-gRNA RNP 442 to second subsequence 420 while inhibiting activity of transposases 432 and 442 in a manner such as described with reference to FIG. 4B (illustratively, presence of a sufficient amount of Ca2+ and/or Mn2+ and absence of a sufficient amount of Mg2+). Method 4000 illustrated in FIG. 4J includes, while promoting activity of the first and second transposases: (i) using the first transposase to add the first amplification adapter to a first location in the target polynucleotide, and (ii) using the second transposase to add the second amplification adapter to a second location in the target polynucleotide (operation 4003). For example, the fluid may have a second condition that promotes activity of first and second transposases 432 and 442 in a manner such as described with reference to FIG. 4C (illustratively, presence of a sufficient amount of Mg2+).

In some implementations, ShCAST (Scytonema hofmanni CRISPR associated transposase) targeted library preparation and enrichment may be used.

Targeted sequencing of specific genes using a separate enrichment step after library preparation may be time-consuming. For example, such a separate enrichment step may involve hybridizing oligonucleotide probes to library DNA and isolating the hybridized DNA on streptavidin-coated beads. Despite significant improvements in efficiency and time required, such separate enrichment protocols may take about two hours and many reagents which can made such protocols challenging to automate.

In comparison, some examples herein may be used to prepare and enrich libraries for targeted sequencing of specific genes, using a single step for both preparation and enrichment.

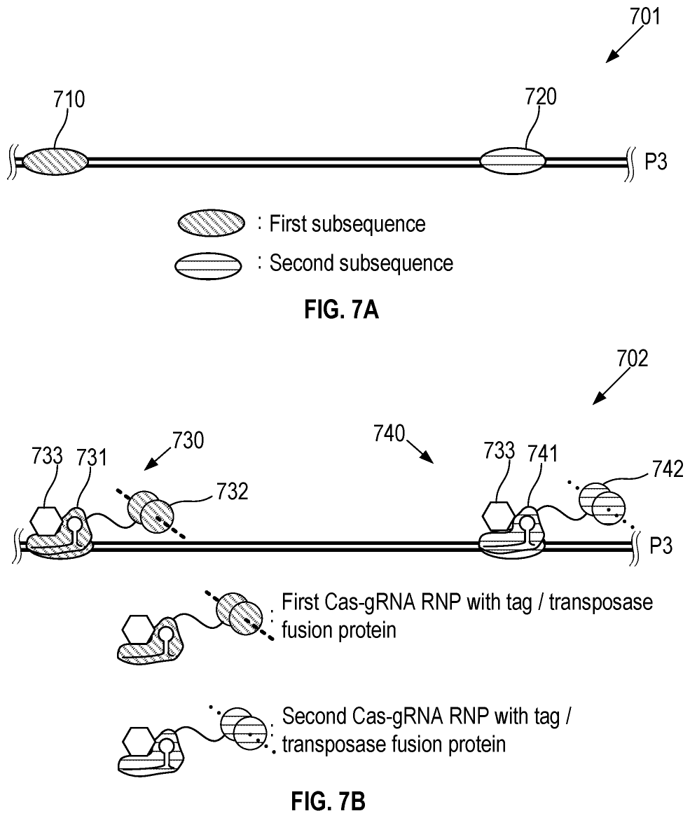

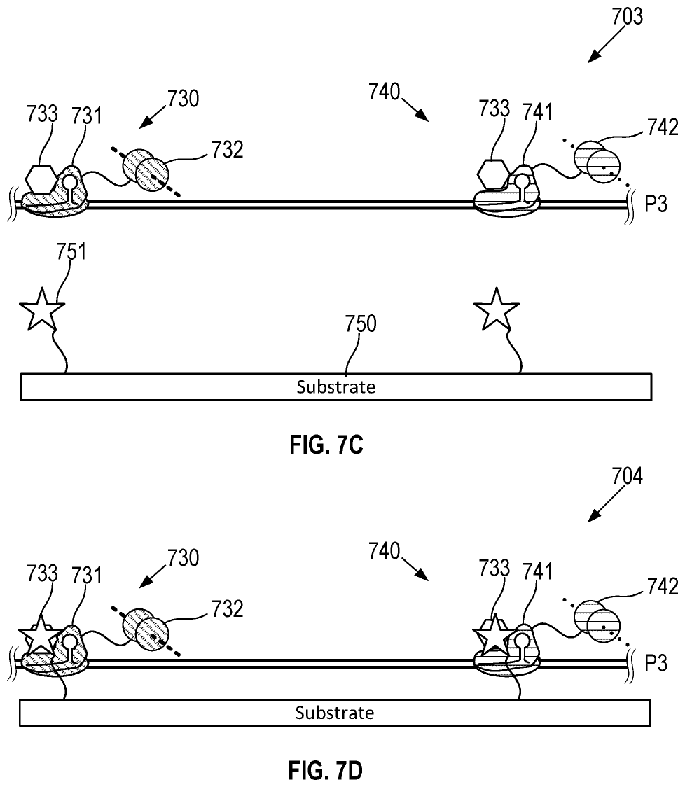

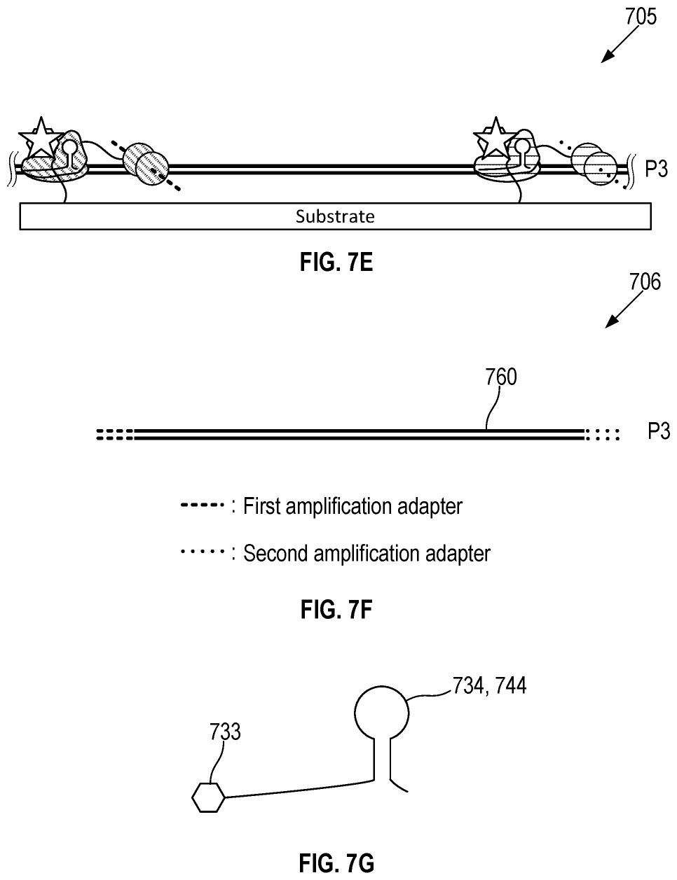

For example, FIGS. 7A-7H schematically illustrate example compositions and operations in another process flow for coupling amplification adapters to polynucleotides. Referring first to FIG. 7A, composition 701 may include a target polynucleotide P3 (such as double-stranded DNA) including a first subsequence 710 that may be targeted using a first Cas-gRNA RNP (that is, include a sequence to which the gRNA of the Cas-gRNA RNP may hybridize). Optionally, composition 701 further may include a second subsequence 720 that may be targeted using a second Cas-gRNA RNP. Target polynucleotide P3 may include partially fragmented dsDNA, such as cell free DNA, or DNA that has been fragmented in a manner such as described elsewhere herein. Alternatively, target polynucleotide P3 may include the DNA of an entire chromosome. As illustrated in FIG. 7B, target polynucleotide P3 may be contacted with first fusion protein 730, and optional second fusion protein 740 in a fluid, in a manner similar to that described with reference to FIGS. 4A-4D. The first fusion protein 730 (and, if present, second fusion protein 740) may be in an approximately stoichiometric ratio to the target polynucleotide P3 in the fluid.

First fusion protein 730 may include first Cas-gRNA RNP 731, which includes tag 733 and is coupled to first transposase 732 having a first amplification adapter (indicated by dashed line) coupled thereto. Optional second fusion protein 740 may include second Cas-gRNA RNP 741, which includes tag 733 and is coupled to second transposase 742 having a second amplification adapter (indicated by dotted line) coupled thereto. Tag 733 may be coupled to any suitable portion of the respective Cas-gRNA RNP in any suitable manner. Non-limiting examples for coupling Cas-gRNA RNPs to transposases are provided further above with reference to FIGS. 4F-4I. It will be appreciated that any suitable amplification adapters may be coupled to target polynucleotide using transposases 732, 742. Illustratively, the first amplification adapter may include a P5 adapter, and the second amplification adapter may include a P7 adapter. Optionally, the first amplification adapter also may include a first unique molecular identifier (UMI), and the second amplification adapter may include a second UMI. The UMIs may be used during sequencing in a manner such as described elsewhere herein.

While promoting activity of first Cas-gRNA RNP 731 (and, if present, second Cas-gRNA RNP 741) and inhibiting activity of first transposase 732 (and, if present, second transposase 742), composition 702 illustrated in FIG. 7B may be provided in which first Cas-gRNA RNP 731 is hybridized to first subsequence 710 in target polynucleotide P3, and, if present, second Cas-gRNA RNP 741 is hybridized to second subsequence 720 in the target polynucleotide. In some examples, activity of first and second Cas-gRNA RNPs 731, 741 may be promoted and the activity of transposases 732, 742 may be inhibited using a condition of the fluid in a manner such as described with reference to FIGS. 4A-4D.

Target polynucleotide P3 may be enriched using tags 733. For example, in composition 703 illustrated in FIG. 7C, target polynucleotide having first and second Cas-gRNA RNPs 731, 732 (respectively coupled to tags 733 and to transposases 732, 742) hybridized thereto, may be brought into contact with substrate 750 coupled to tag partners 751 via respective linkers. Tag partners 751 may be selected so as to covalently or non-covalently couple to tags 733, forming composition 704 such as illustrated in FIG. 7D in which target polynucleotide P3 is coupled to substrate 750 via tags 733 and tag partners 751. Any other polynucleotides that are not coupled to substrate 750 may be washed away.

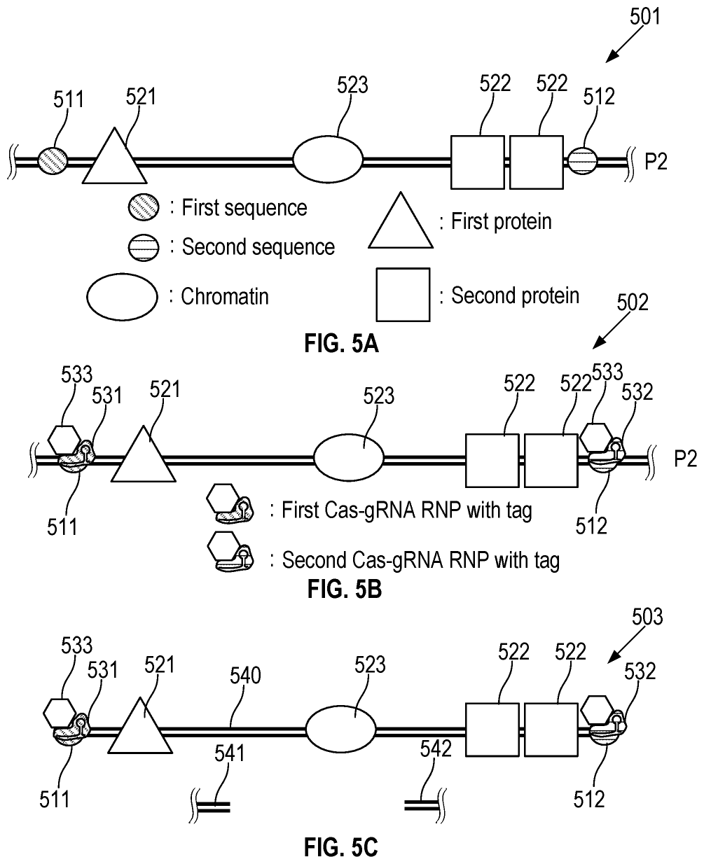

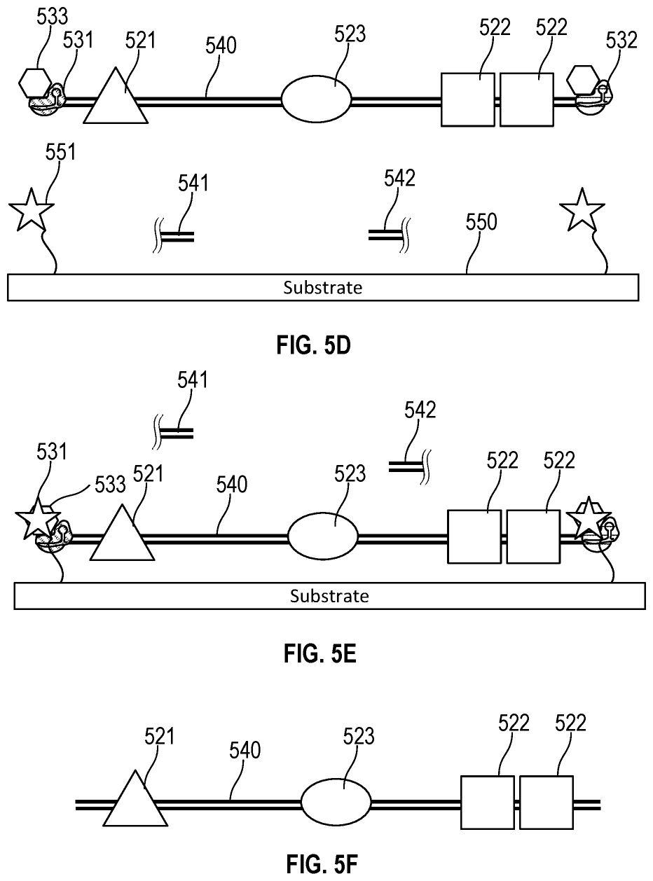

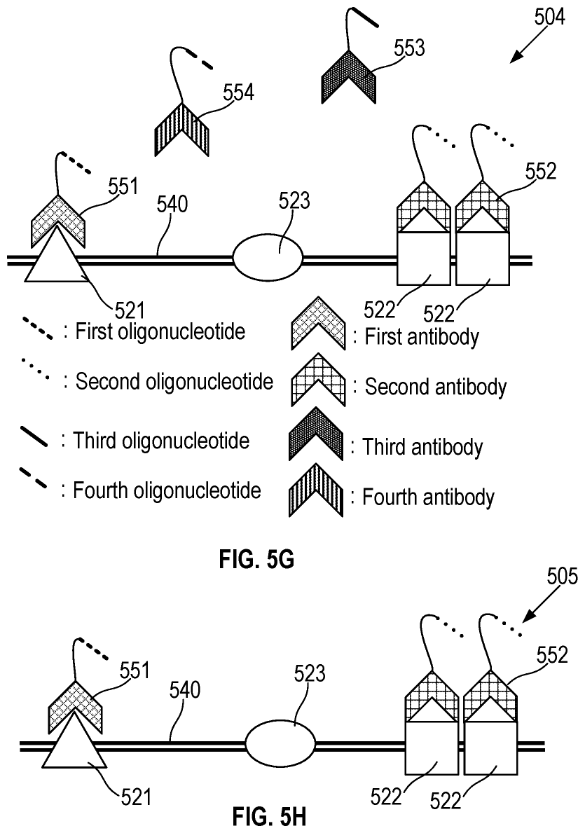

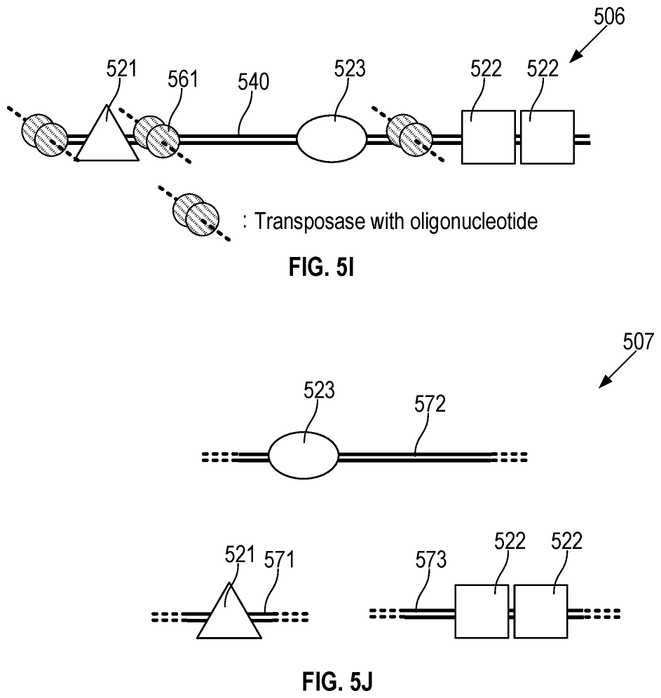

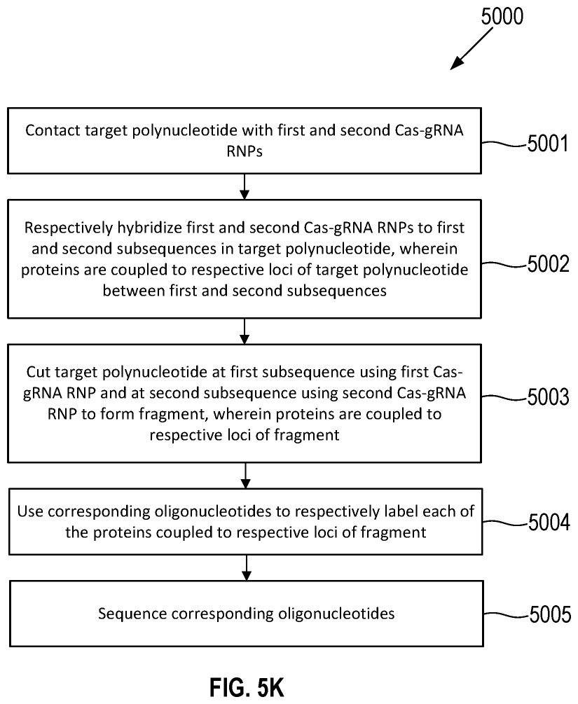

Subsequently, while promoting activity of first and second transposases 732, 742, the first transposase may be used to add the first amplification adapter to a first location in the target polynucleotide P3, and the second transposase may be used to add the second amplification adapter to a second location in the target polynucleotide. For example, activity of transposases 732, 742 may be promoted using a second condition of the fluid, in a manner such as described with reference to FIGS. 4A-4D. As such, composition 705 illustrated in FIG. 7E may be provided in which transposases 732, 742 act upon target polynucleotide P3 to couple the first and second amplification adapters thereto. Polynucleotide P3 may be released from first and second fusion proteins 730, 740 to provide composition 706 illustrated in FIG. 7F which includes fragment 760 of the target polynucleotide P3 having the first amplification adapter at one end, and the second amplification adapter at the other end. Such releasing may be performed using proteinase K, sodium dodecyl sulfate (SDS), or both proteinase K and SDS; by denaturing Cas-gRNA RNPs 731, 741, by decoupling tags 733 from tag partners 751, cleaving linkers between tag partners 751 and substrate 750, or the like. Alternatively, fragment 760 may remain coupled to substrate 750 for subsequent processing. In either example, the resulting enriched fragment 760 illustrated in FIG. 7F (optional coupling to substrate 750 not specifically illustrated) may be further analyzed in a manner such as described with reference to FIGS. 5G-5H or 5I-5J.

Fragment 760 having amplification adapters coupled thereto may be amplified and sequenced. In a manner such as described with reference to FIGS. 4A-4E, the length of fragment 760 may be closely related to, e.g., approximately the distance between, first sequence 710 and second sequence 720. It will be appreciated that fragment 760 illustrated in FIG. 7G may have any suitable length, e.g., as approximately defined by the distance between sequences 710, 720. For example, fragment 760 may have a length of between about 100 base pairs and about 1000 base pairs, e.g., between about 500 base pairs and about 700 base pairs (illustratively, about 600 base pairs), or between about 200 base pairs and about 700 base pairs (illustratively, about 300 base pairs), or between about 100 base pairs and about 200 base pairs (illustratively, about 150 base pairs), or a length of between about 1000 base pairs and about 3000 base pairs (illustratively, about 2000 base pairs).

It will be appreciated that any suitable tags 733 and tag partners 751 may be used to pull down target polynucleotide P3 to substrate 750. For example, tag partners 751 may include SNAP proteins and tags 733 may include O-benzylguanine; the tag partners may include CLIP proteins and the tags may include O-benzylcytosine; the tag partners may include SpyTag and the tags may include SpyCatcher; the tag partners may include SpyCatcher and the tags may include SpyTag; the tag partners may include biotin and the tags may include streptavidin; the tag partners may include streptavidin and the tags may include biotin; the tag partners may include NTA and the tags may include His-Tag; the tag partners may include His-Tag and the tags may include NTA; the tag partners may include antibodies (such as anti-FLAG antibodies) and the tags may include antigens for which the antibodies are selective (such as FLAG tags); the tag partners may include antigens (such as FLAG tags) and the tags may include antibodies that are selective for the antigens (such as anti-FLAG antibodies); or the tag partners may include a first oligonucleotide and the tags may include a second oligonucleotide that is complementary to, and hybridizes to, the first oligonucleotide. Tag partners 751 may be coupled to substrate 750 via any suitable linkage, e.g., via a covalent linkage or via anon-covalent linkage. Similarly, the tags 733 respectively may be coupled to Cas-gRNA RNPs 731, 732 via any suitable linkage, e.g., via a covalent linkage or via a non-covalent linkage, e.g., in a manner similar to that described with reference to FIGS. 4F-4I. In some examples, the gRNA 734, 744 respectively within first and second fusion proteins 730, 740 may be coupled to tag 733 in a manner such as illustrated in FIG. 7G. For example, RNA oligonucleotides coupled to tags may be commercially purchased, and their preparation is known in the art.

It will be appreciated that any suitable Cas and any suitable transposase may be used in fusion proteins 730, 740. Illustratively, the Cas may include dCas9 (e.g., so as to inhibit the Cas from cutting target polynucleotide P3 before the transposase is activated), and the transposase may include Tn5 (e.g., so that the activity of the transposase may be well controlled through fluidic conditions, such as adding as sufficient amount of magnesium ions). In other examples, the Cas may include Cas12k and the transposase may include Tn7 or a Tn7 like transposase (e.g., so that the activity of the transposase may be well controlled through fluidic conditions, such as adding as sufficient amount of magnesium ions). The Cas and transposase may be coupled to one another via any suitable linkage, e.g., via a covalent linkage or via a non-covalent linkage, e.g., in a manner such as described with reference to FIGS. 4F-4I, or in Strecker et al.

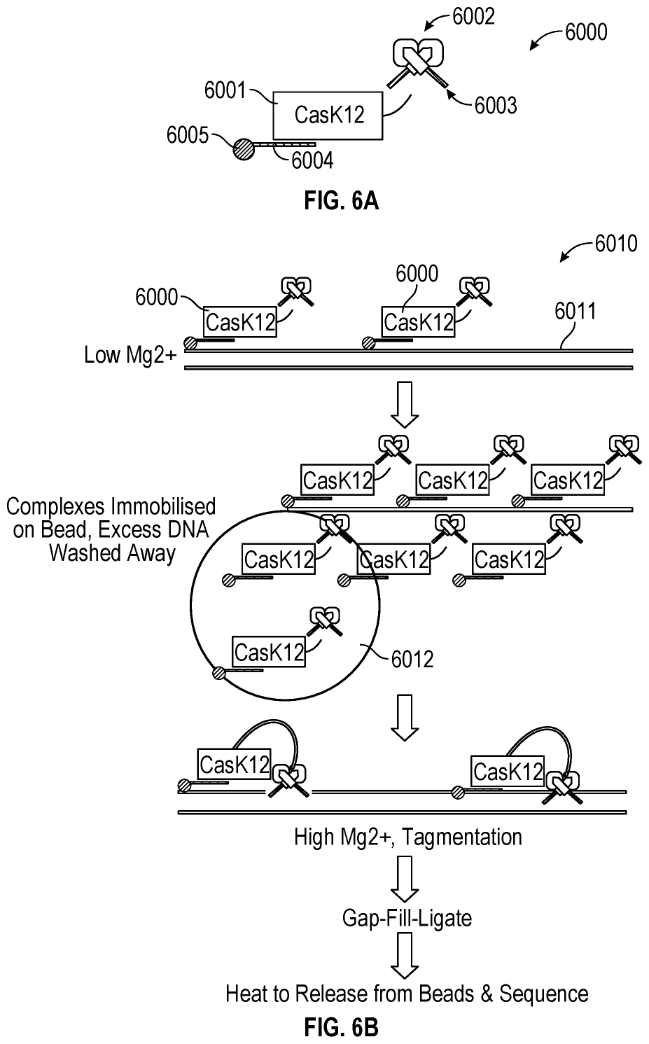

For example, FIGS. 6A-6B schematically illustrate example compositions and operations in a process for ShCAST (Scytonema hofmanni CRISPR associated transposase) targeted library preparation and enrichment. ShCAST 6000 includes Cas12k 6001 and a Tn7-like transposase 6002 that is capable of inserting DNA 6003 into specific sites in the E. coli genome using RNA guides 6004. Some examples provided herein utilize ShCAST or a modified version of ShCAST incorporating a Tn5 transposase (ShCAST-Tn5) for targeted amplification of specific genes. As such, library preparation and enrichment steps are combined, thus simplifying and improving the efficiency of the target library sequencing workflow, and facilitating automation.

Illustratively, gRNA 6004 may be designed to target specific genes (sequences), and the spacing of the gRNAs may control the insert size. In some examples, the gRNA 6004 and/or the ShCAST/ShCAST-Tn5 6002 may be coupled to a tag 6005, e.g., may be biotinylated. In a manner such as illustrated in FIG. 6A, gRNAs 6004 and transposable elements with adapters 6003 (e.g., Illumina adapters) may be loaded onto the transposase 6002 of ShCAST, resulting in complex 6000. In a manner such as illustrated in process flow 6010 of FIG. 6B, the resulting ShCAST/ShCAST-Tn5 complexes 6000 may be mixed with genomic DNA (target polynucleotide) 6011 under fluidic conditions (e.g., low or no magnesium, Mg2+) that inhibit tagmentation, while allowing the complexes to bind to respective sequences in the target DNA, in a manner similar to that described with reference to FIGS. 4A-4J and 7A-7G. The complexes then may be isolated using substrates coupled to tag partners, such as streptavidin beads 6012 to which the tagged (e.g., biotinylated) gRNA and/or ShCAST/ShCAST-Tn5 becomes coupled. Any unbound DNA may be washed away, e.g., to reduce or minimize off-target tagmentation. Then the fluidic conditions may be altered (e.g., sufficiently increasing magnesium) to promote tagmentation, in a manner similar to that described with reference to FIGS. 4A-4J. A gap-fill-ligation step followed by heat dissociation may be used to release the library from beads in preparation for sequencing.

Note that in compositions and operations such as illustrated in FIGS. 6A-6B, the transposase portion 6002 of the complex 6000 may be able to randomly insert into the DNA. Such insertion may be inhibited or minimized by mixing the ShCAST/ShCAST-Tn5 complexes with the genomic DNA under fluidic conditions (e.g., low or no magnesium) that inhibit tagmentation, thus allowing targets to be bound.

For further details regarding ShCAST, including the Cas12k and Tn7 therein, see Strecker et al., “RNA-Guided DNA insertion with CRISPR-associated transposases,” Science 365(6448): 48-53 (2019), the entire contents of which are incorporated by reference herein.

It should be appreciated that tag 733 or tag 6005 may be coupled to the tag partner (and thus to the substrate) at any suitable time, and that such coupling need not necessarily take place after the fusion protein or complex binds to the target polynucleotide, and indeed may take place before the fusion protein or complex binds to the target polynucleotide. Illustratively, gRNA 734, 744, coupled to tag 733 in a manner such as illustrated in FIG. 7G, may be coupled to substrate 750 using an interaction between tag 733 and tag partner 751. The Cas of the fusion protein or complex, which also includes the transposase, then may become coupled to the substrate-bound gRNA. The target polynucleotide then may become coupled to the Cas, thus coupling the target polynucleotide to the substrate.

It also should be appreciated that the process flow described with reference to FIG. 4J may be modified so as to include the use of tags in a manner such as described with reference to FIGS. 6A-6B and 7A-7G. For example, at any suitable time relative to operations 4001 and 4002, tags respectively coupled to the Cas-gRNA RNPs may be used to pull the target polynucleotide down onto a substrate. In a manner such as described with reference to FIGS. 7A-7F, the tags may be coupled to the Cas-gRNA RNPs prior to contacting the polynucleotide with the Cas-gRNA RNPs; alternatively, the tags may be coupled to the gRNA and to the tag partners coupled to the substrate, and the Cas-transposase fusion proteins or complexes brought into contact with the gRNA. Operation 4003 then may be performed so as to promote activity of the transposases and add the amplification adapters to the target polynucleotide.

Accordingly, it may be understood that polynucleotides may be cut at any suitable pairs of locations to form fragments, and any suitable amplification primers may be coupled to the resulting ends of the fragments, using Cas-gRNA RNP/transposase fusion proteins. The fragments then may be amplified and sequenced.

### Compositions and Methods for Targeted Epigenetic Assays

Some examples herein provide for the enrichment of polynucleotides (such as DNA) to generate fragments of epigenetic interest, and assaying proteins at loci along those fragments, using Cas-gRNA RNPs. Several nonlimiting examples of assays are given with specific workflow operations and orderings, but other examples may readily be envisioned. In the present examples, the proteins along a fragment may be labeled using oligonucleotides which subsequently are sequenced, and the oligonucleotides may be used to characterize the proteins. For example, the sequence of the oligonucleotides may provide information about the presence of the proteins at loci of a given fragment, may provide information about the location of the proteins at loci of a given fragment, may provide information about the quantity of the proteins at loci of a given fragment, or any suitable combination of such information. The fragments may be enriched, e.g., specifically selected from a given polynucleotide while other portions of that polynucleotide, and portions of other polynucleotides, may be discarded. Such locus-associated proteome analysis may be used, illustratively, to provide a genome-wide proteomic atlas that complements whole-genome sequencing to provide an enhanced characterization of the relationship between genotype phenotype, or to better characterize epigenetic features associated with specific loci and understand epigenetic mechanisms important for research or for clinical applications and therapies.

For example, FIGS. 5A-5K schematically illustrate example compositions and operations in a process flow for targeted epigenetic assays. As illustrated in FIG. 5A, composition 501 may include a target polynucleotide P2 (such as double-stranded DNA) including a first subsequence 511 that may be targeted using a first Cas-gRNA RNP (that is, include a sequence to which the gRNA of the Cas-gRNA RNP may hybridize), and a second subsequence 512 that may be targeted using a second Cas-gRNA RNP. Target polynucleotide P2 may include a fragment that is generated in a manner such as described in greater detail elsewhere herein, e.g., with reference to FIGS. 2A-2K, 3A-3E, or 4A-4J, or may include an entire chromosome or portion thereof. Proteins 521, 522, and chromatin 523 may be coupled to respective loci of target polynucleotide P2 between the first and second subsequences 511, 512. Optionally, proteins 521, 522 may be cross-linked, e.g., so as to enhance their stability during later processing operations, such as leaving them in place along target polynucleotide P2, while preserving their ability to be selectively targeted by corresponding antibodies in a manner such as described below.

In example composition 502 illustrated in FIG. 5B, target polynucleotide P2 may be contacted with first Cas-gRNA RNP 531 and second Cas-gRNA RNP 532 in a fluid. First Cas-gRNA RNP 531 and second Cas-gRNA RNP 532 each may include a respective tag 533 that may be used to selectively pull down the portion of target polynucleotide P2 between the first and second subsequences 511, 512, thus enriching that portion of the polynucleotide in a manner such as described in greater detail below with reference to FIGS. 5D-5F. In nonlimiting examples, the Cas includes Cas9 or other suitable Cas that may cut target polynucleotide P2. The first and second Cas-gRNA RNPs 531, 532 hybridize to first and second subsequences 511, 512 in target polynucleotide P2, and respectively cut the target polynucleotide at the first and second subsequence to form a fragment. Illustratively, resulting composition 503 illustrated in FIG. 5C includes fragment 540 having one first protein 521, two second proteins 522, and chromatin 523 coupled to respective loci thereof, as well as first and second Cas-gRNA RNPs 531, 532 respectively coupled to tags 533 and respectively hybridized to subsequences 511, 512, thus coupling tags 533 to fragment 540. Fragment 540 may have any suitable length, e.g., between about 100 base pairs and about 1000 base pairs, such as between about 500 base pairs and about 700 base pairs, or between about 200 base pairs and about 400 base pairs, or between about 100 base pairs and about 200 base pairs, or a length of between about 1000 base pairs and about 3000 base pairs (illustratively, about 2000 base pairs). Remaining portions 541, 542 of polynucleotide P2 may have any length, and in some examples may form the balance of the chromosome after removal of fragment 540.

Fragment 540 may be enriched using tags 533. For example, as illustrated in FIG. 5D, fragment 540 having first and second Cas-gRNA RNPs 531, 532 (respectively coupled to tags 533) hybridized thereto, as well as remaining portions 541, 542 of polynucleotide P2, may be brought into contact with substrate 550 coupled to tag partners 551 via respective linkers. Tag partners 551 may be selected so as to covalently or non-covalently couple to tags 533, forming a composition such as illustrated in FIG. 5E in which fragment 540 is coupled to substrate 550 via tags 533 and tag partners 551, while remaining portions 541, 542 are not coupled to substrate 550 and may be washed away. Fragment 540 then may be released from substrate 550, e.g., by denaturing Cas-gRNA RNPs 531, 532 (in which case proteins 521, 522 may have been previously cross-linked to inhibit their denaturation), by decoupling tags 533 from tag partners 551, cleaving linkers between tag partners 551 and the substrate, or the like. Alternatively, fragment 540 may remain coupled to substrate 550 for subsequent processing. In either example, the resulting enriched fragment 540 illustrated in FIG. 5F (optional coupling to substrate 550 not specifically illustrated) may be further analyzed in a manner such as described with reference to FIGS. 5G-5H or 5I-5J.

It will be appreciated that any suitable tags 533 and tag partners 551 may be used to pull down fragment 540. For example, tag partners 551 may include SNAP proteins and tags 533 may include O-benzylguanine; the tag partners may include CLIP proteins and the tags may include O-benzylcytosine; the tag partners may include SpyTag and the tags may include SpyCatcher; the tag partners may include SpyCatcher and the tags may include SpyTag; the tag partners may include biotin and the tags may include streptavidin; the tag partners may include streptavidin and the tags may include biotin; the tag partners may include NTA and the tags may include His-Tag; the tag partners may include His-Tag and the tags may include NTA; the tag partners may include antibodies (such as anti-FLAG antibodies) and the tags may include antigens for which the antibodies are selective (such as FLAG tags); the tag partners may include antigens (such as FLAG tags) and the tags may include antibodies that are selective for the antigens (such as anti-FLAG antibodies); or the tag partners may include a first oligonucleotide and the tags may include a second oligonucleotide that is complementary to, and hybridizes to, the first oligonucleotide. The tags 533 respectively may be coupled to Cas-gRNA RNPs 531, 532 via any suitable linkage, e.g., via a covalent linkage or via a non-covalent linkage, e.g., in a manner similar to that described with reference to FIGS. 4F-4I or FIG. 7G. Similarly, tag partners 551 may be coupled to substrate 550 via any suitable linkage, e.g., via a covalent linkage or via a non-covalent linkage.

As provided herein, corresponding oligonucleotides may be used to respectively label each of the proteins 521, 522 coupled to the respective loci of the fragment (which fragment may be prepared and enriched in a manner such as described in a manner such as described with reference to FIGS. 5A-5F), and such oligonucleotides then may be sequenced. The proteins may be identified, the loci may be identified, and/or the proteins may be quantified, using the corresponding oligonucleotides.

In some examples that now will be explained with reference to FIGS. 5G-5H, using corresponding oligonucleotides to respectively label each of the proteins may include contacting enriched fragment 540 with a mixture of antibodies that are specific to different proteins, each of the antibodies being coupled to a corresponding oligonucleotide that may be used to label the protein in such a manner as to characterize the protein. For example, composition 504 illustrated in FIG. 5G includes enriched fragment 540 in contact with a plurality of each of first, second, third, and fourth antibodies 551, 552, 553, 554 which respectively are coupled to corresponding first, second, third, and fourth oligonucleotides. Each of antibodies 551, 552, 553, 554 is specific to a different protein. It will be appreciated that enriched fragment 540 may be contacted with any suitable number and type of different antibodies that are specific to different proteins or other chromatin that potentially may be coupled to loci along that fragment and that may be of epigenetic interest. For any antibodies in the mixture that are specific to the proteins coupled to the respective loci of enriched fragment 540, those antibodies and the corresponding oligonucleotides may become non-covalently coupled to those proteins via antibody/target binding. In the nonlimiting example composition 505 illustrated in FIG. 5E, first antibody 551 is specific to, and is coupled to, first protein 521, while second antibody 552 is specific to, and is coupled to, second protein 522. Note that a plurality of second proteins 522 are coupled to a respective one of the loci, and a plurality of second antibodies 552 in the mixture are coupled to the proteins at that locus. In this example, enriched fragment 540 does not include the proteins for which third and fourth antibodies 553, 554 are specific, and so those antibodies (and their respective oligonucleotides) do not become coupled to the fragment.

Custom oligonucleotide-conjugated antibodies are commercially available, or may be prepared using known techniques, e.g., such as described in the following references, the entire contents of each of which are incorporated by reference herein: Gong et al., “Simple method to prepare oligonucleotide-conjugated antibodies and its application to multiplex protein detection in single cells,” Bioconjugate Chem. 27: 217-225 (2016); and Stoeckius et al., “Simultaneous epitope and transcriptome measurement in single cells,” Nature Methods 14: 865-868 (2017).

The first and second oligonucleotides that respectively are coupled to antibodies 551, 552 may be sequenced and respectively used to identify the presence, and optionally the quantity, of proteins 521, 522 within enriched fragment 540. In some examples, the first and second oligonucleotides may be released from fragment 540, e.g., by applying a protease that digests proteins 521, 522 and antibodies 551, 552, and then amplified and sequenced. Such sequencing may be performed in any suitable manner. For example, sequencing the corresponding oligonucleotides may include hybridizing the corresponding oligonucleotides to a bead array, e.g., using Illumina BeadArray™ technology (San Diego, CA), or performing sequencing-by-synthesis (SBS) on the corresponding oligonucleotides. The oligonucleotides optionally may include amplification adapters (e.g., P5 and P7 adapters, or Y-shaped adapters) and/or UMIs, or such amplification adapters and/or UMIs may be added to the oligonucleotides using known techniques such as PCR, prior to amplification and sequencing.

Regardless of the particular sequencing method used, the respective presences of the corresponding oligonucleotides may be used to identify, and optionally quantify, the proteins coupled to enriched fragment 540. For example, the presence of the first and second oligonucleotides may be detected using the bead array or SBS, and based upon such presence it may be deduced that the first and second proteins 521, 522 were present in fragment 540. Respective quantities of the corresponding oligonucleotides also may be used to quantify the proteins. For example, because enriched fragment 540 included two second proteins 522, two copies of second antibody 552 became coupled thereto, together with two copies of the second oligonucleotide, in comparison to the one first protein 521 which become coupled to one copy of first antibody 551 and one copy of the first oligonucleotide. The relative quantities of the first oligonucleotide (one copy) and the second oligonucleotide (two copies) indicate the relative quantities of the first protein 521 (one copy) and second protein 522 (two copies) within enriched fragment 540. The absence of the third and fourth oligonucleotides indicate that the proteins for which the third and fourth antibodies 553, 554 respectively are selective were not present in enriched fragment 540. Accordingly, the present methods provide for the assaying of epigenetic features of enriched fragment 540, more specifically of proteins that are coupled to loci along enriched fragment 540.

In other examples, which now will be explained with reference to FIGS. 5I-5J, using corresponding oligonucleotides to respectively label each of the proteins may include contacting a fragment with a plurality of transposases, each of the transposases being coupled to a corresponding oligonucleotide that may be used to label a protein in such a manner as to characterize the protein. For example, composition 506 illustrated in FIG. 5I includes enriched fragment 540 (which may be prepared in a manner such as described with reference to FIGS. 5A-5F) in contact with a plurality of transposases 561 that respectively include oligonucleotides. In nonlimiting examples, the transposases may include Tn5.

The proteins coupled to the respective loci of the enriched fragment may inhibit activity of the transposases at the loci. As such, the transposases 561 may become coupled to fragment 540 at locations other than the loci. At the locations at which the transposases 561 are coupled to fragment 540, the transposases may couple the corresponding oligonucleotides to the fragment. Such process may divide the fragment 540 into subfragments. In the nonlimiting example composition 507 illustrated in FIG. 5J, subfragment 571 includes first protein 521 and the oligonucleotide, subfragment 572 includes chromatin 523 and the oligonucleotide, and subfragment 573 includes proteins 522 and the oligonucleotide. In this regard, note that because transposases 561 (illustrated in FIG. 5I) may become coupled to fragment 540 at any location that is not inhibited by the presence of proteins 521, 522 or chromatin 523 (that is, are not specific to a given protein or portion of the fragment), such transposases may add their respective oligonucleotides to any such locations.

The oligonucleotides that respectively are coupled to second, first, and third fragments 571, 572, 573 may be sequenced and respectively used to identify the presence, and optionally the quantity, of proteins 521, 522 and chromatin 523, e.g., in a manner such as described with reference to FIGS. 5G-5H. Respective locations in fragments 571, 572, 573 of the oligonucleotides may be used to identify the respective locus of proteins and/or chromatin. For example, in the purely illustrative view shown in FIGS. 5I and 5J, protein 521 inhibits any transposase from acting at the locus of that protein, proteins 522 inhibit any transposase from acting at the locus of those proteins, and chromatin 523 inhibits any transposase from acting where that chromatin is located. As such, the respective locations of the proteins 522, 521 and/or chromatin 523 in the second, first, and third oligonucleotides in fragments 572, 571, 573 may be understood to be at locations other than where the oligonucleotides were added.

FIG. 5K illustrates an example flow of operations in a method 5000 of characterizing proteins coupled to respective loci of a target polynucleotide. Method 5000 may include contacting the target polynucleotide with first and second Cas-gRNA RNPs (operation 5001), e.g., in a manner such as described with reference to FIGS. 5A-5C. Optionally, method 5000 may include enriching the fragment before using the corresponding oligonucleotides to respectively label each of the proteins coupled to the respective loci of the fragment. For example, the first and second Cas-gRNA RNPs respectively may be coupled to tags such that the fragment is coupled to the tags via the first and second Cas-gRNA RNPs, e.g., in a manner such as described with reference to FIGS. 5B-5C. The enriching may include contacting the fragment, coupled to the tags via the first and second Cas-gRNA RNPs, with a substrate coupled to tag partners, e.g., in a manner such as described with reference to FIG. 5D. The enriching further may include coupling the tags to the tag partners to couple the fragment to the substrate, e.g., in a manner such as described with reference to FIG. 5E. The enriching further may include removing any portions of the target polynucleotide that are not coupled to the substrate, e.g., in a manner such as described with reference to FIG. 5F.

Method 5000 may include respectively hybridizing the first and second Cas-gRNA RNPs to first and second subsequences in the target polynucleotide, wherein proteins are coupled to respective loci of the target polynucleotide between the first and second subsequences (operation 5002), e.g., in a manner such as described with reference to FIGS. 5A-5C. Method 5000 may include cutting the target polynucleotide at the first subsequence using the first Cas-gRNA RNP and at the second subsequence using the second Cas-gRNA RNP to form a fragment, wherein the proteins are coupled to respective loci of the fragment (operation 5003), e.g., in a manner such as described with reference to FIGS. 5A-5C. Method 5000 may include using corresponding oligonucleotides to respectively label each of the proteins coupled to the respective loci of the fragment (operation 5004) and sequencing the corresponding oligonucleotides (operation 5005), e.g., in a manner such as described with reference to FIGS. 5G-5H, and/or in a manner such as described with reference to FIGS. 5I-5J.

It will be appreciated that the process flows such as respectively described with reference to FIGS. 5G-5H and 5I-5J may be performed using any suitable length of polynucleotide, and need not necessarily be performed using a fragment which has been generated using process flows such as described with reference to FIGS. 5A-5C. As such, operations 5001-5003 of method 5000 described with reference to FIG. 5K should be understood to be optional.

Accordingly, from FIGS. 5A-5K it may be understood that in some examples herein, Cas-gRNA RNPs may be used to generate and enrich polynucleotide fragments that are coupled to proteins, and that the locations, quantities, and/or identities of those proteins may be characterized using epigenetic assays such as described herein.

### Enriching Selected Fragments of Polynucleotides Using Cas-gRNA RNP Nickases

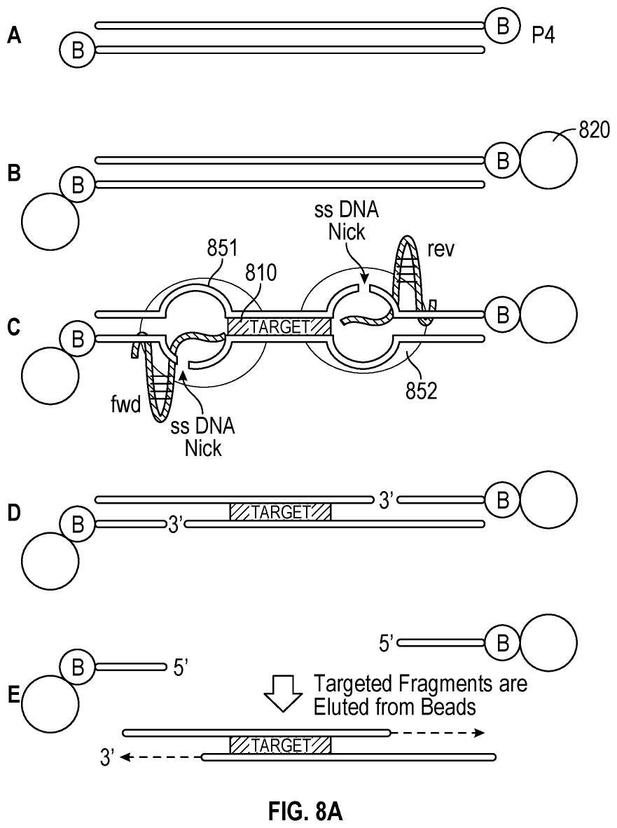

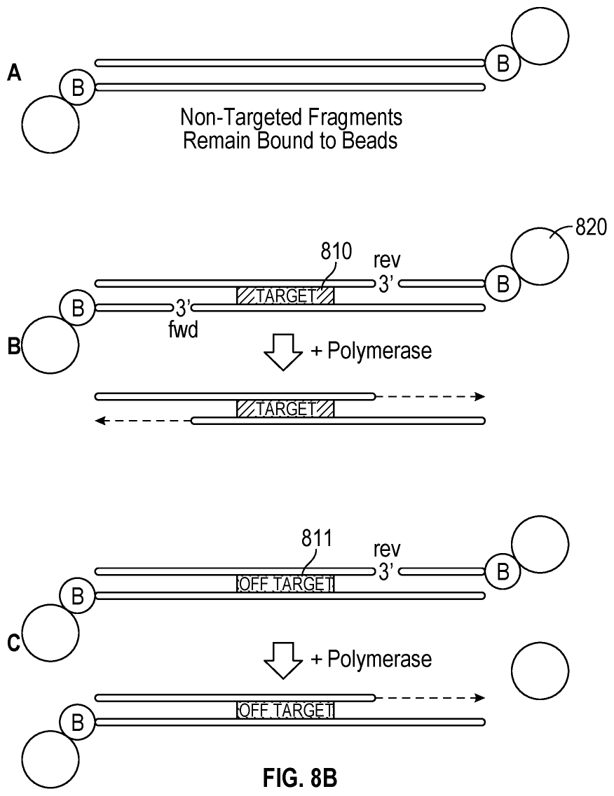

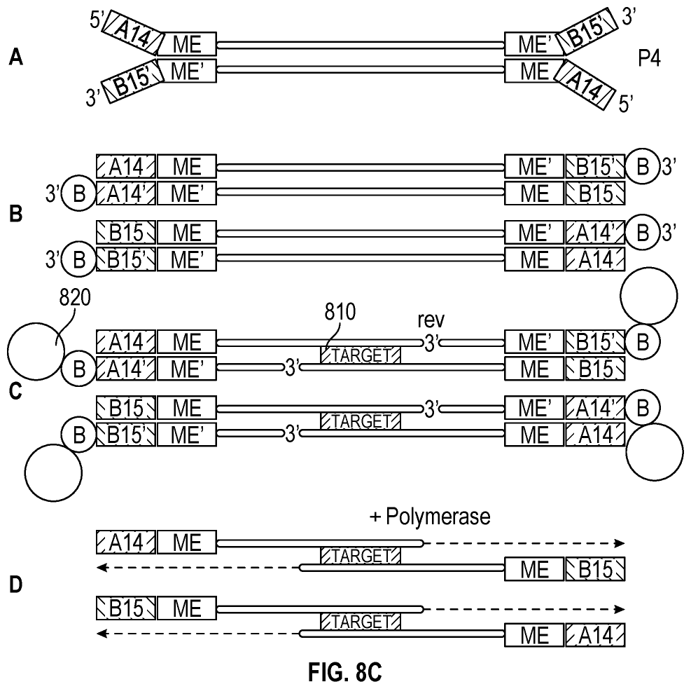

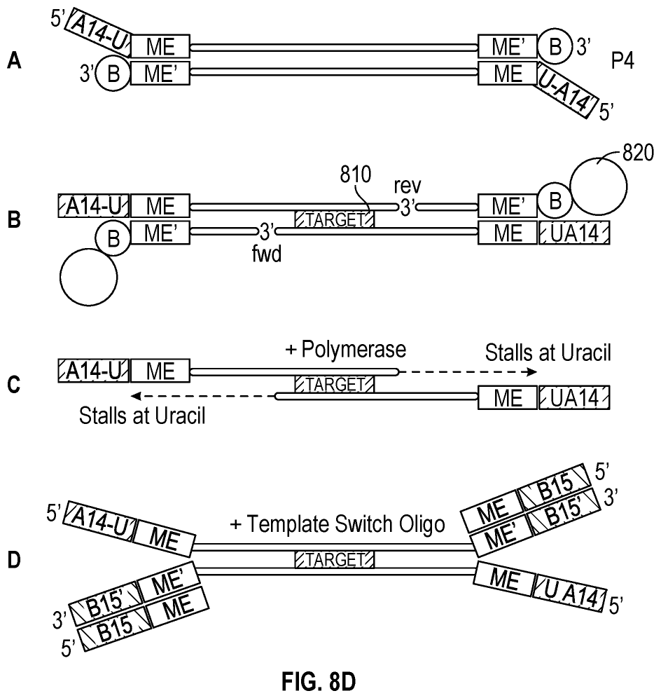

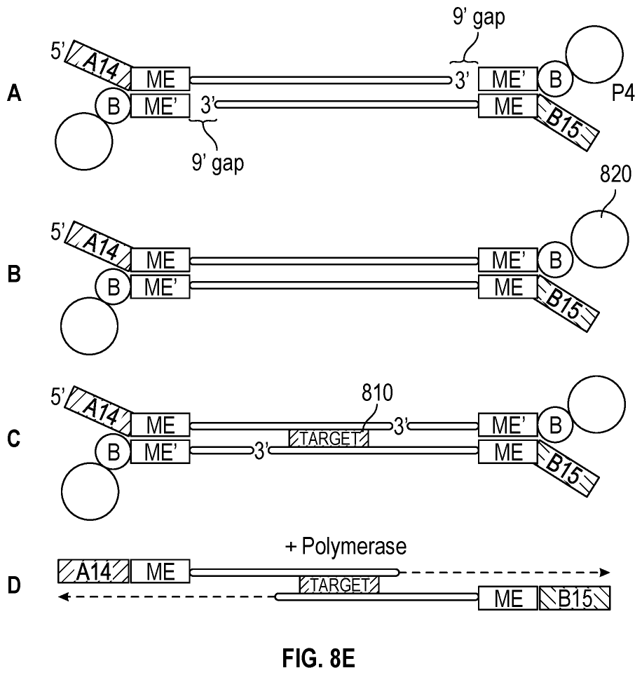

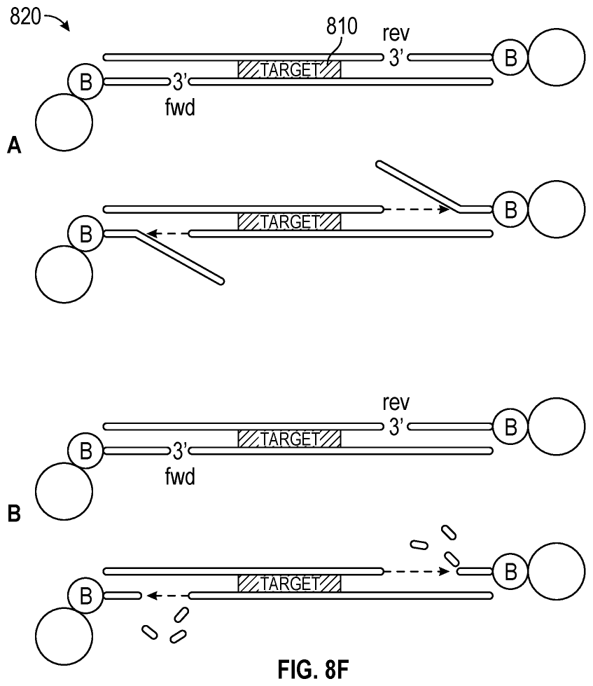

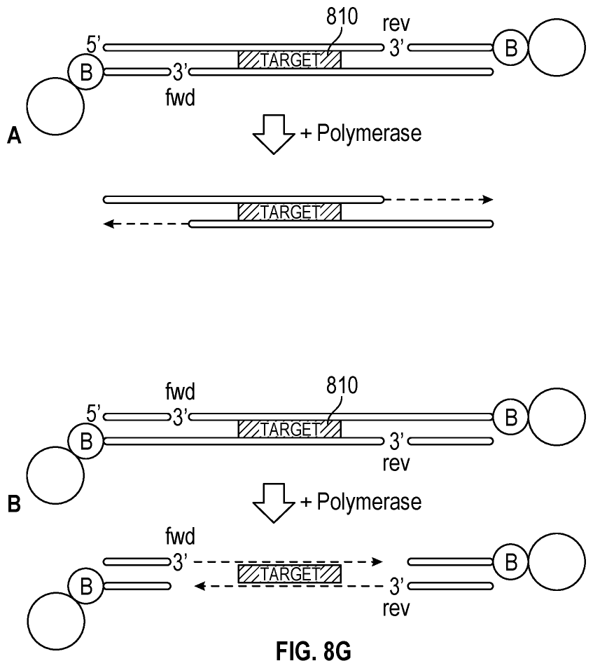

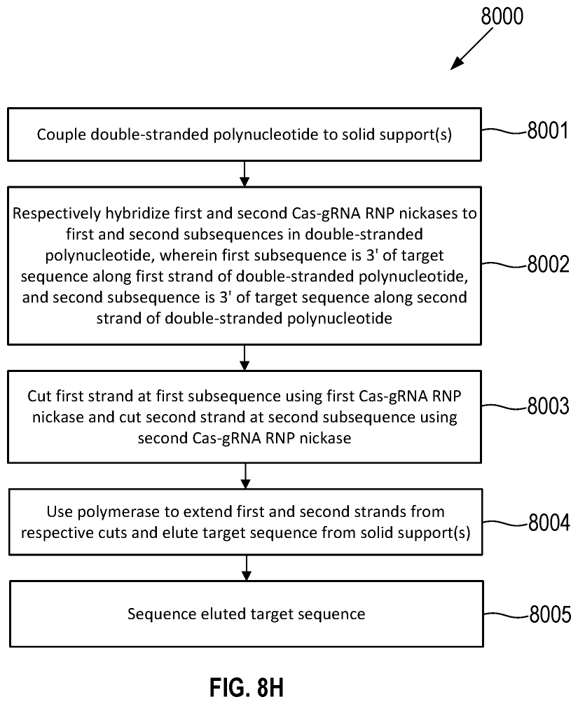

Some methods provided herein solve the problem of long and laborious workflows for targeted sequencing of intact dsDNA fragments. As will be clear from the present disclosure, Cas-gRNA RNPs may provide for rapid and specific cleavage of target regions in polynucleotides, e.g., dsDNA. As now will be described with reference to FIGS. 8A-8H, Cas-gRNA RNP nickases and polymerase extension may be used to selectively enrich dsDNA fragments through elution from a substrate. Such methods and compositions may be used to recover intact originating fragments. This may be particularly useful in applications where full dsDNA cleavage by Cas-gRNA RNPs may be undesirable, e.g., in sequencing cell free DNA (cfDNA). This may also or alternatively be useful as the underlying size of the sequencing library is not being changed by the CRISPR cleavage, which means reducing or avoiding the generation of very short products.

More specifically, FIGS. 8A-8H schematically illustrate example compositions and operations in a process flow for enriching selected polynucleotide fragments using Cas-gRNA RNP nickases. FIG. 8A illustrates an overview of an example process flow for CRISPR nickase extension for selective elution of target regions. At operation A of the process flow, dsDNA fragments P4 (which optionally may be generated in a manner such as described elsewhere herein) may be 3′ functionalized (“B”) to as to facilitate coupling the fragments to beads. For example, the fragments may be 3′ biotinylated using a method such as described below with reference to FIG. 8C. Some of the fragments P4 may include respective target sequence(s) that it is desired to enrich and detect, while other fragments may not necessarily include such sequence(s); for example, the fragment P4 illustrated in FIG. 8A includes target sequence 810, while other fragments may include other target sequences or may not include any such target sequences.

At operation B of the process flow illustrated in FIG. 8A, the 3′ functionalized fragments P4 may be coupled to one or more substrates, e.g., beads that are functionalized in such a manner as to become coupled to the 3′ functionalized fragments P4. In one nonlimiting example, beads 820 may include streptavidin to which 3′ biotinylated fragments P4 become coupled. In the illustrated example, each of the functionalized 3′ ends of the dsDNA fragment P4 becomes coupled to a different bead 820, but it will be understood that in other examples the 3′ functionalized ends of a given fragment P4 may become coupled to the same bead as one another. The beads 820 may be pulled out of solution (e.g., the beads may be ferromagnetic or paramagnetic and may be pulled out of solution using an external magnet), and the beads then washed to provide purified dsDNA fragments P4 coupled to the beads, while any other dsDNA substantially may be washed away.

As illustrated in FIG. 8A, at operation C the bead-coupled fragments P4 may be contacted with a plurality of Cas-gRNA RNP nickases (also referred to herein as CRISPR nickases). The gRNA of each of the Cas-gRNA RNP nickases may target a specific region (subsequence) within a respective single strand of the dsDNA, and the regions may be staggered so that the nickases cut respective strands at locations that are offset from one another and that are on opposing sides of a double-stranded target region 810 that it is desired to enrich. For example, in a manner such as illustrated in operation C of FIG. 8A, the gRNA of first Cas-gRNA RNP nickase 851 may target a region that is forward (“fwd”) of target sequence 810, and the gRNA of second Cas-gRNA RNP nickase 852 may target a region that is reverse (“rev”) of target sequence 810. As such, the guide sequences of first and second nickases 851, 852 may be considered to “flank” target sequence 810 in the forward and reverse directions. The first Cas-gRNA RNP nickase 851 creates a nick (cut) in one strand of the bead-coupled dsDNA fragment P4, and the second Cas-gRNA RNP nickase 852 creates a nick in the other strand of the bead-coupled dsDNA fragment P4 at a location that is offset from that created by nickase 851. It will be appreciated that any suitable number of gRNAs may be designed to direct corresponding Cas-gRNA RNP nickases to cut respective strands at locations that flank specific sequences within dsDNA fragments. For example, multiple different gRNAs (e.g., 1000-100,000 gRNAs, or more than 100,000 gRNAs) may be used so as to simultaneously enrich for many different sequences of interest in a sample. Note that the gRNAs need not necessarily “flank” a given target sequence 810, but rather that at least two guides per target sequence may bind and create nicks on opposing strands within a given fragment P4.

As illustrated in operation D of FIG. 8A, the Cas-gRNA RNP nickases 851, 852 are removed so as to expose 3′ ends of the nicks, for example using mild heat and/or reagents to destroy the Cas-gRNA RNP nickases, such as Proteinase K, proteases, or SDS detergent. Because the strands of each given dsDNA fragment P4 remain hybridized to one another, the fragment remains substantially coupled to corresponding bead(s) 820.

The target sequence 810, which is flanked by opposing nicks in the strands of the dsDNA, then selectively may be eluted into solution while remaining portions of fragment P4 remain coupled to bead(s) 820. For example, nicked fragment P4 may be contacted with polymerase and nucleotides (not specifically illustrated). In a manner such as illustrated in operation E of FIG. 8A, the polymerase may extend the respective strands of the fragment from the 3′ ends exposed by the nicks, and such extension may displace the bound strands, resulting in elution of target sequence 810. Non targeted regions remain coupled to bead(s) 820 and separated from the eluted target sequence 810, e.g., using magnetic or other separation techniques. The polymerase extension results in elution of the intact sequence 810, regardless of where the nicks occurred within fragment P4.

### Example Workflow for Enriching Targets in Lambda DNA Using a Cas9 Nickase and a Polymerase Extension to Elute from a Substrate

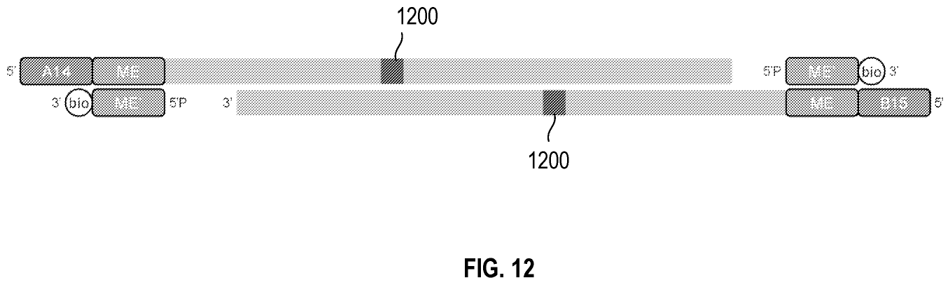

FIG. 8A illustrates an exemplary workflow that can be used to enrich targets in lambda DNA using a Cas9 nickase. Specific guide RNA sequence that target four regions of the lambda genome are used. FIGS. 12-16 provides schematics of the library structures after various steps of the workflow, as described in more detail below. Table 1 provides the guide RNA sequences as well as the regions they target.

TABLE 1
Guide RNA Sequences

| | Lambda | Guide sequence | |
|---|---|---|---|
| Region | cut | (target | Full guide |
| code | site | portion) | RNA sequence |
| region1+ | 192 | TTTGTCCGTG | mU*mU*mU*rGrUrC |
| | | GAATGAACAA | rCrGrUrGrGrArAr |
| | | | UrGrArArCrArArG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region1− | 323 | GGCATACCAT | mG*mG*mC*rArUrA |
| | | TTTATGACGG | rCrCrArUrUrUrUr |
| | | | ArUrGrArCrGrGrG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region2+ | 15024 | TTACATGAGA | mU*mU*mA*rCrArU |
| | | CTCTGCCTGA | rGrArGrArCrUrCr |
| | | | UrGrCrCrUrGrArG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region2− | 15160 | GGTCATACCA | mG*mG*mU*rCrArU |
| | | CCGGCCCCAA | rArCrCrArCrCrGr |
| | | | GrCrCrCrCrArArG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region3+ | 35292 | GCGCTTACCC | mG*mC*mG*rCrUrU |
| | | CAACCAACAG | rArCrCrCrCrArAr |
| | | | CrCrArArCrArGrG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region3− | 35372 | CACCACCAAA | mC*mA*mC*rCrArC |
| | | GCTAACTGAC | rCrArArArGrCrUr |
| | | | ArArCrUrGrArCrG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region4+ | 25073 | TGTCCTATAT | mU*mG*mU*rCrCrU |
| | | CACCACAAAA | rArUrArUrCrArCr |
| | | | CrArCrArArArArG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |
| region4− | 25176 | AGTAGTTGGT | mA*mG*mU*rArGrU |
| | | AACCTGACAA | rUrGrGrUrArArCr |
| | | | CrUrGrArCrArArG |
| | | | rUrUrUrUrArGrAr |
| | | | GrCrUrArGrArArA |
| | | | rUrArGrCrArArGr |
| | | | UrUrArArArArUrA |
| | | | rArGrGrCrUrArGr |
| | | | UrCrCrGrUrUrArU |
| | | | rCrArArCrUrUrGr |
| | | | ArArArArArGrUrG |
| | | | rGrCrArCrCrGrAr |
| | | | GrUrCrGrGrUrGrC |
| | | | mU*mU*mU*rU |

The Cas9 enzyme is loaded with guide RNA sequences. The guide sequences are loaded separately onto Cas9 in a final volume of 50 uL containing 1 uM guide, 1 uM Cas9 nickase (Integrated DNA Technologies, Alt-R® S.p. Cas9 D10A Nickase V3, 1081062) and 1× phosphate buffered saline. The components are left at room temperature for 10 minutes and then pooled in equal volumes to make the Cas9 nicking mix. The solution is stored on ice until use.

Library Prep by Tagmentation with Bead-Linked Transposomes

Libraries attached to a small surface by the 3′ end were prepared using bead-linked transposomes.

Step 1: 500 ng of Lambda DNA were incubated with 10 uL TB1 and 10 uL of eBLT from the Illumina DNA Prep with Enrichment kit in a total volume of 50 uL. The mixture was heated to 41° C. for 5 min.

Step 2: Tn5 was removed by adding 10 uL of ST2 and heated at 37° C. for 5 min.

FIG. 12 shows the library structure after step 2. Element 1200 shows the PAM site in the DNA insert.

Step 3: The reaction plate was placed on a magnetic stand and the beads were allowed to pellet. The supernatant was removed, and the beads were washed by adding 150 uL of TWB. The magnet was then removed, and the solution was mixed through pipetting. The beads were pelleted on the magnet again, after which the magnet was removed. The supernatant was discarded.

Step 4: 50 uL of ELM (from the Illumina DNA Prep PCR Free kit) was added to the solution. The solution was incubated at 37° C. for 15 minutes to gap fill and ligate between the 3′ end of the insert and the non-transferred strand of the transposon.

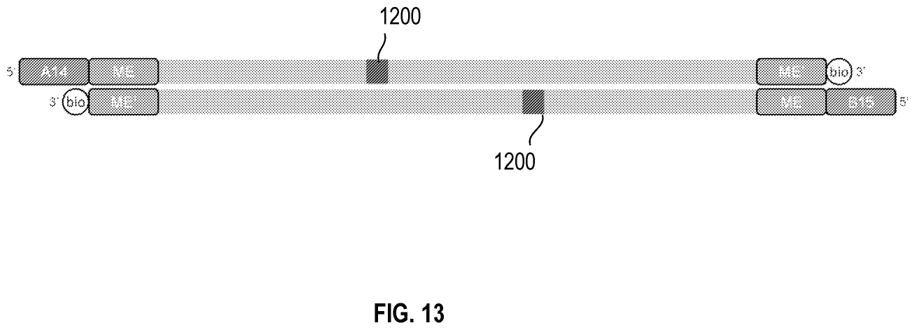

FIG. 13 shows the library structure after Step 4. Element 1200 shows the PAM site in the DNA insert.

Step 5: The beads are pelleted on the magnet. The supernatant was removed and washed with TWB.

Step 6: Any incompletely gap filled and ligated fragments that could contribute to background were removed by adding 0.5 uL of exonuclease III (New England Biolabs, M0206) in 1× NEBuffer 1 (New England Biolabs) in a 50 uL volume. The beads were resuspended by pipette mixing and heating to 37° C. for 10 minutes.

### Cas9 Nicking Reaction

Step 1: The supernatant was removed by adding 2 uL of the pooled, loaded Cas9 nickase with 1× NEBuffer 2.1 (New England Biolabs) in a total volume of 20 uL. The beads were resuspended by pipette mixing and heating to 37° C. for 30 minutes.

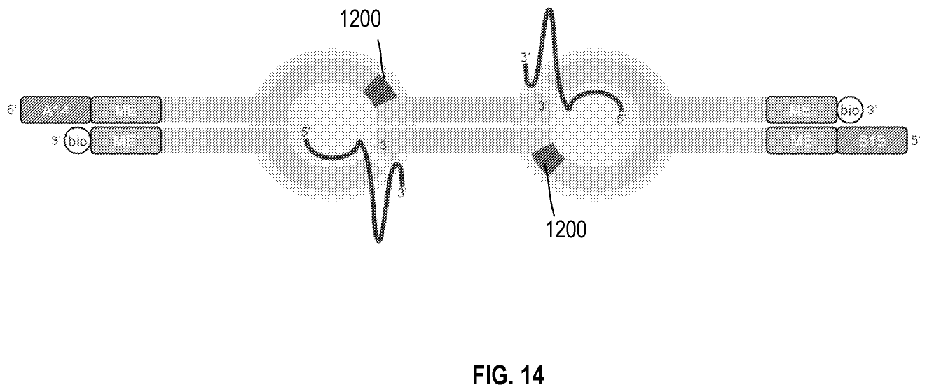

FIG. 14 shows how the Cas9 nicks a target fragment on each strand.

Step 2: The Cas9 was removed by adding 10 uL ST2 and heating to 37° C. for 5 minutes. The beads were pelleted and washed twice with TWB. The supernatant was discarded.

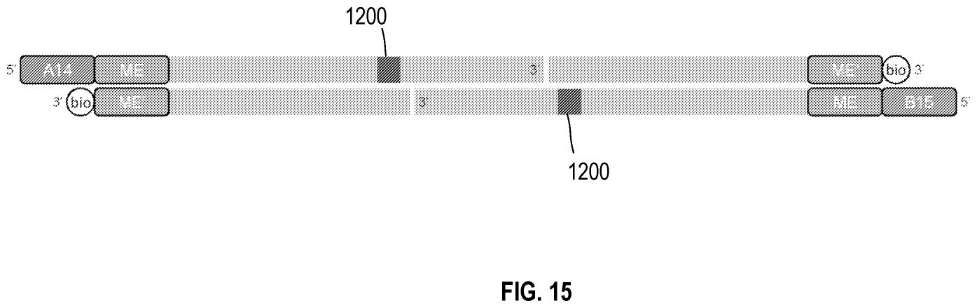

FIG. 15 shows the library structure at this point, with one nick in each strand. Element 1200 shows the PAM site in the DNA insert.

Polymerase Extension to Elute Target Fragments from the Beads

0.5 uL of DNA polymerase I (New England Biolabs, M0210) or Bsu DNA polymerase (New England Biolabs, M0330) was added to the solution. A 1× NEBuffer 2 (New England Biolabs) was used, and 200 uM of each dNTP was added in a total volume of 50 uL. The solution was heated to 37° C. for 10 minutes.

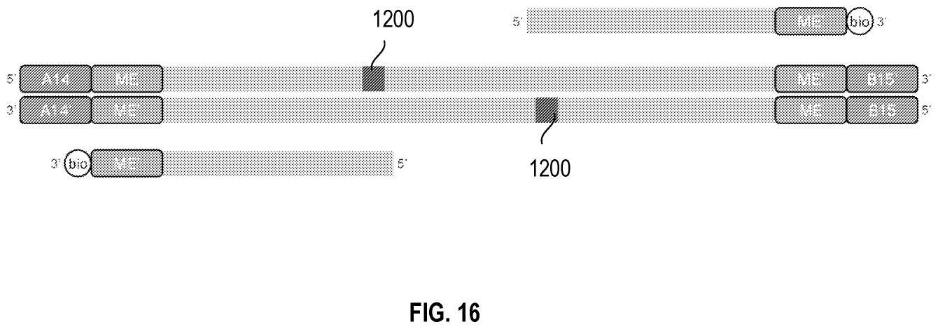

FIG. 16 shows that after polymerase extension. As shown, the fragments no longer have a 3′ biotin and are therefore released into solution. Element 1200 shows the PAM site in the DNA insert.

### Purification and PCR

Step 1: The beads were pelleted and 40 uL of the supernatant containing the selected target fragments were transferred into a new tube. The beads were purified using Illumina Purification Beads (IPB) by adding 100 uL of ITB, mixing well, and incubating at room temperature for 5 minutes. The beads were pelleted on a magnet and washed twice with 180 uL of 80% ethanol. The supernatant was removed and allowed to dry for 2 minutes and then resuspended in 27 uL of water. The solution was mixed well, and the beads were pelleted and 25 uL of the supernatant was transferred to a new tube.

Step 2: The libraries were amplified by adding 20 uL EPM and 5 uL of an indexing primer mix, using the following PCR program:
98° C. for 1 min
12 cycles of 98° C. for 20 seconds
60° C. for 30 seconds
72° C. for 30 seconds
cool to 10° C.

### Sequencing

The libraries were quantified using a Qubit kit (dsDNA BR Assay Kit, Thermo Scientific) and fluorometer and then sequenced on a MiSeq at 12 pM loading concentration.

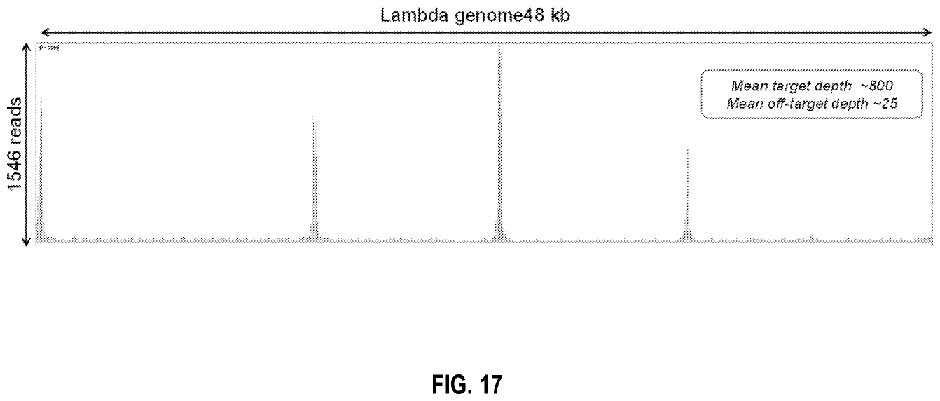

FIG. 17 shows sequencing depth across the lambda genome after enrichment of the four targets.

FIG. 8B illustrates further details regarding use of at least two CRISPR events on a fragment P4 to cause selective elution such as described with reference to FIG. 8A. At operation A illustrated in FIG. 8B, no nicking events have occurred and fragment P4 therefore remains coupled to bead(s) 820. At operation B illustrated in FIG. 8B, two nicking events have occurred in a manner such as described with reference to operation C described with reference to FIG. 8A, and it may be seen that subsequent extension of the nicks using a polymerase displaces both of the ends which were coupled to respective beads, thus eluting target sequence 810. At operation C illustrated in FIG. 8C, only a single nicking event has occurred, e.g., because a Cas-gRNA RNP nickase created a nick at an off-target sequence 811 of fragment P4 and therefore a corresponding nickase did not create a nick at an opposing portion of fragment P4 that flanked sequence 811. It may be seen that although subsequent 3′ extension of the nick using a polymerase displaces one of the ends which was coupled to a respective bead, the other end remains coupled to a bead and therefore is not eluted. As such, it may be understood from FIG. 8B that fragments which are nicked on opposing strands on either side of a target sequence 810 may be eluted preferentially relative to fragments that are not nicked, or that are nicked on only a single strand. Note that the gRNAs may be designed to become coupled to regions at which the corresponding nickases may generate nicks at respective positions that are 3′ of the target sequence 810 and thus may successfully be eluted using polymerase extension, whereas any nicks generated at positions that are 5′ of the target sequence may be unable to extend past nicks on the template strand, e.g., in a manner such as described in greater detail below with reference to FIG. 8G. Note that the Cas-gRNA RNP nickases optionally may target different strands. Although the figures may illustrate a single nickase that targets the strand hybridized with the gRNA, another nickase may be used which nicks the other strand. This may provide for improved choice of sequences for nicking as both strands in the genome may be used.

FIG. 8C illustrates an example process flow for enriching dsDNA fragments from a sequencing library that has undergone PCR amplification prior to nicking and extension operations such as described with reference to FIGS. 8A-8B. Such PCR amplification may be useful for enhancing sensitivity and/or to amplify enough material to perform quality control and to sequence from small panels, for example if the Cas-gRNA RNP nickase binding and nicking steps are not 100% efficient and/or if there is a relatively low number of dsDNA fragments, e.g., if the dsDNA is obtained from a cell-free DNA (cfDNA) sequencing library. At operation A illustrated in FIG. 8C, an amplification adaptor is added via any suitable method, e.g., such as described with reference to FIGS. 1J, 3D, 4A-4J, 6A-6B, or 7A-7G. The amplification adaptors optionally may be Y-shaped in a manner such as described with reference to FIGS. 1J and 3D, and may provide read 1 and read 2 sequencing primers respectively. In one nonlimiting example, the amplification adaptors may include A14 and B15 amplification adaptors (the complements to which are A14′ and B15′) in addition to a double stranded ME, ME′ region. For example, as illustrated in FIG. 8C operation A, the 3′ end of a first strand of dsDNA fragment P4 may be coupled to a B15′ amplification adaptor via an ME′ sequence, and the 5′ end of that strand may be coupled to an A14 amplification adaptor via an ME sequence. The 3′ end of a second strand of fragment P4 may be coupled to a B15′ amplification adaptor via an ME′ sequence, and the 5′ end of that strand may be coupled to an A14 amplification adaptor via an ME sequence. However, it will be appreciated that any other sequences and/or amplification adaptors may be added to the strands, e.g., UMIs, sample indexes, cluster amplification primers, and the like.

Following preparation of the library with amplification adapters, PCR amplification is carried out to separately amplify both strands of the initial fragments P4, as illustrated in operation B of FIG. 8C. During, or at the end of, this operation, the fragments may be functionalized (e.g., biotinylated) at the 3′ end in a manner similar to that described with reference to operation A of FIG. 8A. Illustratively, non-template addition (e.g., using Taq polymerase) or terminal transferase may be used to add biotinylated nucleotides to the 3′ ends of the amplified strands as illustrated in operation B of FIG. 8C. Subsequent operations may be performed similarly as described with reference to FIG. 8A. For example, as illustrated in operation C of FIG. 8C, the whole library may be coupled to one or more substrates (such as bead(s) 820) via the 3′ functional groups of fragments P4 in a manner such as described with reference to operation B of FIG. 8A, and Cas-gRNA RNP nickases used to generate nicks that 3′ flank respective target sequences 810 in a manner such as described with reference to operation C of FIG. 8A. As illustrated in operation D of FIG. 8C, the Cas-gRNA RNP nickases then may be removed in a manner such as described with reference to operation D of FIG. 8A, and polymerase added to extend from the nicks and cause elution of the target sequence 810 in such a manner such as described with reference to operation E of FIG. 8A. The eluted target sequences then may be further amplified, e.g., using PCR or cluster amplification, during which amplification UMIs, sample indexes, and/or clustering adaptors optionally may be added, e.g., if such sequence(s) were not added during operation A of FIG. 8C. Nonlimiting examples of sample indexes include Illumina i5 and i7 indexes. Nonlimiting examples of clustering adaptors include P5 and P7 primers. The eluted fragments, having any suitable sequences coupled thereto, may be sequenced on any suitable platform (e.g., an Illumina sequencing-by-synthesis platform) as part of a targeted sequencing assay.

It will be appreciated that while PCR may be used to couple suitable adaptors to fragments P4 and to amplify the fragments prior to Cas-gRNA mediated elution, PCR need not necessarily be used as such. For example, FIG. 8D illustrates a process flow for enriching fragments from a PCR-free fragmented and ligated sequencing library. Here, at operation A of FIG. 8D, fragments P4 are generated and an amplification adaptor (e.g., ME/ME′ regions and a 5′ amplification adaptor) is added via any suitable method, e.g., such as described with reference to FIGS. 1J, 3D, 4A-4J, 6A-6B, or 7A-7G. In the nonlimiting example illustrated in FIG. 8D, a 3′ functional group (such as biotin) may be added through adaptor ligation, e.g., using a simplified adaptor that includes ME/ME′ and a single A14 adaptor. The adaptor may be modified so as to include uracil (U) which may stall polymerase extension in a manner such as described further below with reference to operations C and D of FIG. 8D. At operation B of FIG. 8D, the 3′ functional groups are coupled to substrates, such as beads 820, in a manner such as described with reference to operation B of FIG. 8A, and Cas-gRNA RNP nickases used to generate nicks that 3′ flank respective target sequences 810 in a manner such as described with reference to operation C of FIG. 8A. As illustrated in operation C of FIG. 8D, the Cas-gRNA RNP nickases then may be removed in a manner such as described with reference to operation D of FIG. 8A, and polymerase added to extend from the nicks and cause elution of the target sequence 810 in such a manner such as described with reference to operation E of FIG. 8A. However, the uracil within the modified adaptors (e.g., A14-U) causes the polymerase to stall at the location of that uracil. As illustrated in operation C of FIG. 8D, a template switch oligonucleotide including a second sequencing primer (e.g., B15) allows for the stalled extension product to prime off and append a 3′ amplification adaptor to the eluted target fragments. The eluted target fragments 810 optionally then may be PCR amplified, including the addition of cluster amplification adaptors (e.g., P5 and P7), UMIs, and/or sample indexes, in a manner such as described elsewhere herein. However, it will be appreciated that a PCR-free process flow suitably may be implemented, e.g., by adding full sequencing/cluster amplification adaptors and sample indexes at operations A and D of the process flow illustrated in FIG. 8D. For further details regarding selected operations described with reference to FIG. 8D, see International Patent Publication No. WO 2021/252617, entitled “Methods for Increasing Yield of Sequencing Libraries,” the entire contents of which are incorporated by reference herein.

It will be appreciated that process flows such as described with reference to FIGS. 8A-8D suitably may be adapted for use with any type of library, instrument, or workflow. FIG. 8E illustrates a nonlimiting example of a process flow for use with an Illumina Nextera workflow. Here, a sample library may be prepared through simultaneous fragmentation and 5′ adaptor addition using a Nextera system. Nextera systems may be bound to a substrate (e.g., bead(s) 820) in a manner such as described with reference to operation B of FIG. 8A, so that the initial fragmentation event may be used to couple the fragment P4 to the substrate. As illustrated in operation A of FIG. 8E, a Nextera library may be generated that is coupled to bead(s) 820, e.g., via 3′ functional groups such as biotin. In some examples, the library may be generated using a mixture of transposomes including respective amplification adaptors, such as A14 and B15 adaptors, in which case a portion (e.g., about half) of the fragments P4 may include A14 and B15 adaptors at either end (as shown here). Other fragments may not necessarily include both A14 and B15 adaptors, e.g., may lack a B15 adaptor but include two A14 adaptors, or may lack an A14 adaptor but include two B15 adaptors.

As a result of the Nextera fragmentation process, each fragment P4 may include gaps between the 3′ end and the ME region that are about 9 base pairs long. As illustrated in operation B of FIG. 8E, the gaps may be sealed, for example by extension ligation using polymerase and ligase. Note that sealing the nicks may inhibit any non-specific extension and elution with the polymerase. Alternatively, a terminated base may be added to inhibit unwanted extension and elution later with TdT or a polymerase, and a dideoxy base. Then, in a similar manner as described with reference to operation C of FIG. 8A, Cas-gRNA RNP nickases may be applied to the fragments on the substrate(s), creating targeted nicks flanking target sequences 810 in a manner such as illustrated in operation C of FIG. 8E. Then, in a similar manner as described with reference to operation E of FIG. 8A, polymerase may be added to cause the target sequences to elute, in a manner such as illustrated in operation D of FIG. 8E. Following elution, further amplification adaptors and/or sample indexes may be coupled to the fragments in a manner such as described elsewhere herein, e.g., using PCR or cluster amplification prior to sequencing. In this regard, any fragments P4 that have two B15 adaptors or two A14 adaptors may not be amplified during such PCR amplification, and accordingly may not be sequenced. It will be appreciated that a template switch mechanism, such as described with reference to operation D of FIG. 8D, may be used to reduce the loss of such B15-B15 fragments and A14-A14 fragments by replacing adaptors so as to provide both A14 and B15 adaptors, so that such fragments may be amplified using PCR or cluster amplification, and subsequently sequenced.

FIG. 8F illustrates polymerase options for nick extension elution operations such as described with reference to operation E of FIG. 8A, operation B of FIG. 8B, operation D of FIG. 8C, operation C of FIG. 8D, and operation D of FIG. 8E. At example A of FIG. 8F, use of a strand displacing polymerase results in displacement of the 3′ functionalized (e.g., 3′ biotinylated) strand from the target sequence 810, resulting in targeted elution. At example B of FIG. 8F, a nick translation approach including use of a polymerase with 5′ exonuclease activity causes 5′ to 3′ degradation of the 3′ functionalized (e.g., 3′ biotinylated) strand, resulting in targeted elution of the target sequence 810.

FIG. 8G compares the use of nicks that are 3′ of the target sequence (operation A) to the use of nicks that are 5′ of the target sequence (operation B). As may be understood from operation A, two nicking events that are 3′ of the target sequence 810 results in elution of the target sequence form the substrate(s), e.g., bead(s) 820. As may be understood from operation B, two nicking events that are 5′ of the target sequence 810 may cause the polymerase to stall at the nicks, causing the target sequence to remain bound to the substrate(s), e.g., bead(s) 820.

Note that numerous separation techniques are compatible with process flows such as described with reference to FIGS. 8A-8G, and are not limited to the use of magnetic separation of beads such as described. For example, the substrate(s) may be provided within a flow system, such as a packed column or flowcell. Target fragments may be eluted using flow in such systems.

It will further be appreciated that the fragments P4 may be functionalized to include any suitable tags, and that the substrate(s) may be functionalized to include any suitable tag partners for pulling down the fragments P4 to the substrate(s). For example, the tag partners may include SNAP proteins and the tags may include O-benzylguanine; the tag partners may include CLIP proteins and the tags may include O-benzylcytosine; the tag partners may include SpyTag and the tags may include SpyCatcher; the tag partners may include SpyCatcher and the tags may include SpyTag; the tag partners may include biotin and the tags may include streptavidin; the tag partners may include streptavidin and the tags may include biotin; the tag partners may include NTA and the tags may include His-Tag; the tag partners may include His-Tag and the tags may include NTA; the tag partners may include antibodies (such as anti-FLAG antibodies) and the tags may include antigens for which the antibodies are selective (such as FLAG tags); the tag partners may include antigens (such as FLAG tags) and the tags may include antibodies that are selective for the antigens (such as anti-FLAG antibodies); or the tag partners may include a first oligonucleotide and the tags may include a second oligonucleotide that is complementary to, and hybridizes to, the first oligonucleotide. The tag partners may be coupled to the substrate via any suitable linkage, e.g., via a covalent linkage or via a non-covalent linkage. Similarly, the tags respectively may be 3′ coupled to the fragments P4 via any suitable linkage, e.g., via a covalent linkage or via a non-covalent linkage.

Compositions and operations such as described with reference to FIGS. 8A-8G may be used in any suitable method or context. For example, FIG. 8H illustrates a flow of operations in an example method 8000 of generating a fragment of a double-stranded polynucleotide. Although method 8000 may describe operations that are performed on a particular polynucleotide, it will be appreciated that the method may be applied to a mixture that includes several different polynucleotides which may be operated upon concurrently in the described manner. In some examples, the double-stranded polynucleotide may include dsDNA, and optionally may include cfDNA.

Method 8000 may include coupling the double-stranded polynucleotide to a substrate (operation 8001). For example, in a manner such as described with reference to operation A of FIG. 8A, operation B of FIG. 8C, operation A of FIG. 8D, or operation A of FIG. 8E, the 3′ ends of the double-stranded polynucleotide may be functionalized, e.g., may be coupled to a tag or tag partner. Additionally, in a manner such as described with reference to operation B of FIG. 8A, operation C of FIG. 8C, operation B of FIG. 8D, or operation A of FIG. 8E, the 3′ functionalized ends of the double-stranded polynucleotide may be coupled to a substrate, e.g., a substrate that is coupled to a tag partner or tag that becomes coupled to the tag or tag partner of the double-stranded polynucleotide. While some examples described with reference to FIGS. 8A-8G may include streptavidin beads as a substrate and biotin as the 3′ functional group, many other examples of substrates and tag/tag partner pairs readily may be envisioned.

Method 8000 illustrated in FIG. 8H also may include respectively hybridizing first and second CRISPR-associated protein guide RNA ribonucleoprotein (Cas-gRNA RNP) nickases to first and second subsequences in the double-stranded polynucleotide (operation 8002). The first subsequence may be 3′ of a target sequence along a first strand of the double-stranded polynucleotide, and the second subsequence may be 3′ of the target sequence along a second strand of the double-stranded polynucleotide. For example, the gRNA of a first Cas-gRNA RNP nickase 851 selectively may become coupled to a first strand of the double-stranded polynucleotide P4 at a “fwd” 3′ location, and the gRNA of a second Cas-gRNA RNP nickase 852 selectively may become coupled to a second strand of the double-stranded polynucleotide P4 at a “rev” 3′ location in a manner such as described with reference to operation C of FIG. 8A, operation B of FIG. 8B, operation C of FIG. 8C, operation B of FIG. 8D, operation C of FIG. 8E, example A of FIG. 8F, example B of FIG. 8F, and example A of FIG. 8G. The nickases may be considered to 3′ “flank” the target sequence. As noted above, the nickases may target either the gRNA-hybridized strand or the opposite strand.

Method 8000 illustrated in FIG. 8H also may include cutting the first strand at the first subsequence using the first Cas-gRNA RNP nickase, and cutting the second strand at the second subsequence using the second Cas-gRNA RNP nickase (operation 8003). For example, the nickase of the first Cas-gRNA RNP nickase 851 selectively may nick the first strand of the double-stranded polynucleotide P4 at a location defined by the subsequence to which the gRNA of that nickase becomes coupled, and the nickase of the second Cas-gRNA RNP nickase 852 selectively may nick the second strand of the double-stranded polynucleotide P4 at a location defined by the subsequence to which the gRNA of that nickase becomes coupled, in a manner such as described with reference to operation C of FIG. 8A, operation B of FIG. 8B, operation C of FIG. 8C, operation B of FIG. 8D, operation C of FIG. 8E, example A of FIG. 8F, example B of FIG. 8F, and example A of FIG. 8G. The resulting cuts may be considered to 3′ “flank” the target sequence. Such two instances of cutting may be performed concurrently, or may occur at different times than one another. For example, a large pool of first and second strand CRISPR nickase complexes may be incubated with the sample at once. It will be appreciated that operations 8002 and 8003 may be performed using any suitable Cas-gRNA RNP nickases, illustratively S. pyogenes Cas9 with a first mutation D10A and a second mutation H840A.

Method 8000 also may include using a polymerase to extend the first and second strands from the respective cuts and elute the target sequence from the substrate (operation 8004). For example, the Cas-gRNA RNP nickases may be removed to expose the 3′ ends of the nicks generated in operation 8003, and a suitable polymerase added to extend the target sequence, which is double-stranded, from the 3′ ends, in a manner such as described with reference to operations D and E of FIG. 8A, operation B of FIG. 8B, operation D of FIG. 8C, operation C of FIG. 8D, operation D of FIG. 8E, example A of FIG. 8F, example B of FIG. 8F, and example A of FIG. 8G. Such extension displaces the portions of the double-stranded polynucleotide that are coupled to the substrate, which remain bound to the substrate, and elutes the target sequence. Accordingly, the target sequence is released from the substrate. It will be appreciated that operation 8004 may be performed using any suitable polymerase. For example, the polymerase may include a strand displacement polymerase such as described with reference to example A of FIG. 8F, illustratively Vent or Bsu. Or, for example, the polymerase may have 5′ exonuclease activity, illustratively Taq, Bst, or DNA Polymerase I.

Method 8000 also may include sequencing the eluted target sequence (operation 8005). Such sequencing may be performed in any suitable manner and using any suitable instrument, e.g., an instrument that is commercially available from Illumina, Inc. At any suitable time prior to sequencing, the target sequence suitably may be coupled to amplification adaptors, e.g., in a manner such as described with reference to operations A through D of FIG. 8C, operations A through D of FIG. 8D, or operations A through D of FIG. 8E. Such amplification adaptors may be added before or after any suitable ones of operations 8001, 8002, 8003, and 8004. Additionally, at any suitable time prior to sequencing, the target sequence may be amplified, e.g., using PCR or cluster amplification. Such amplification may be performed before or after any suitable ones of operations 8001, 8002, 8003, and 8004.

### Ligating Amplification Adaptors to Selected Polynucleotide Fragments Using Cas-gRNA RNPs

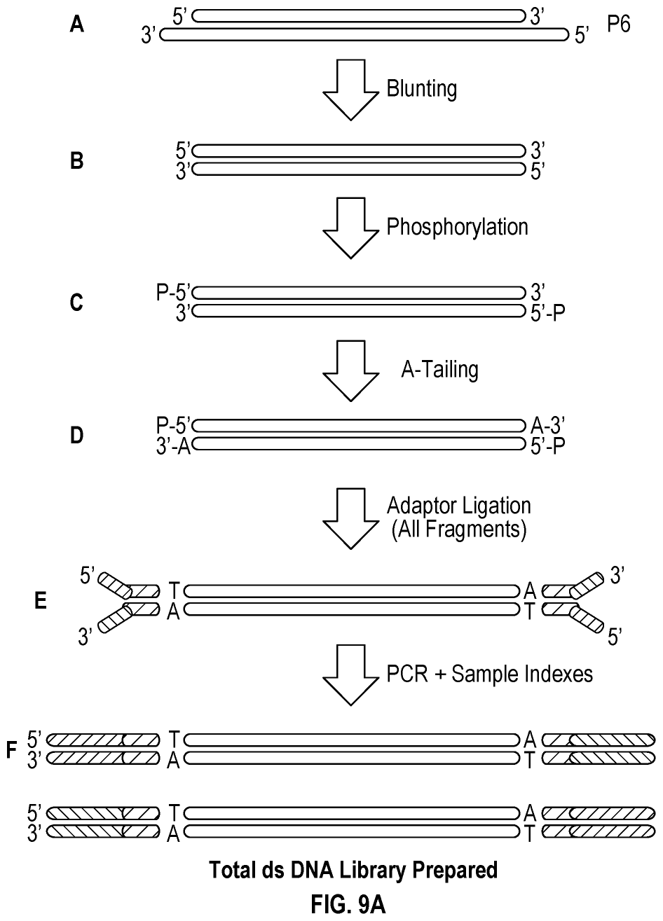

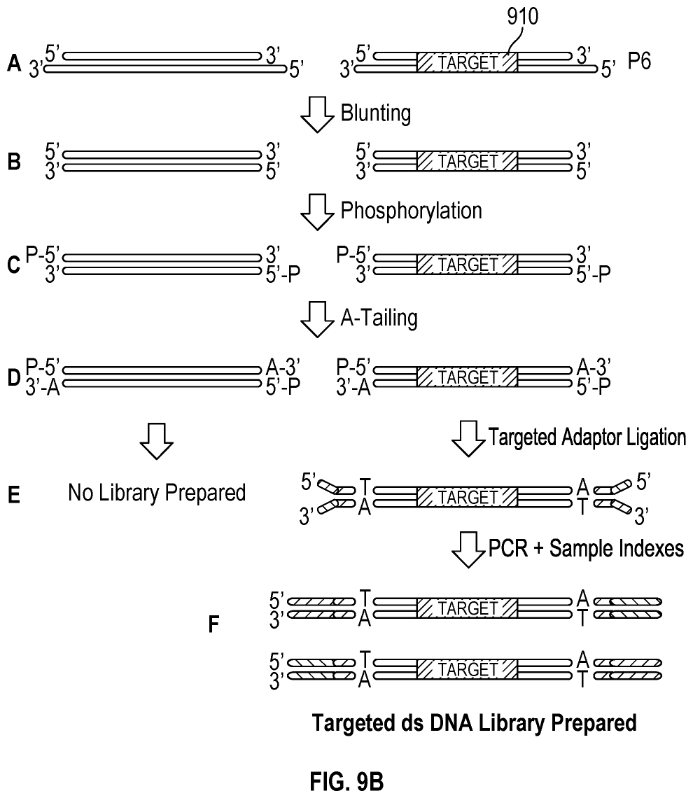

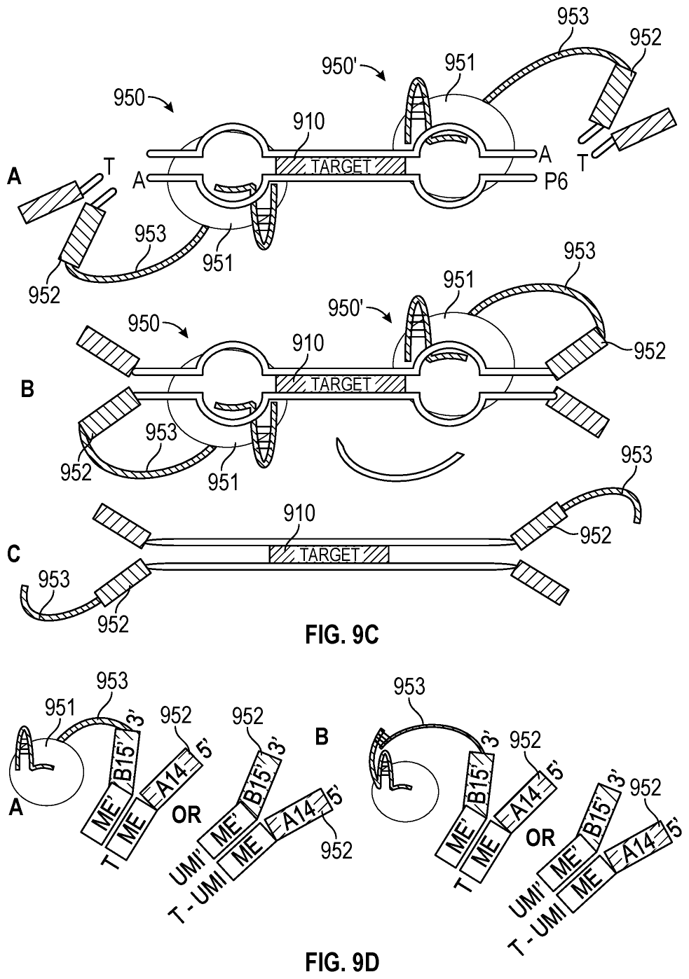

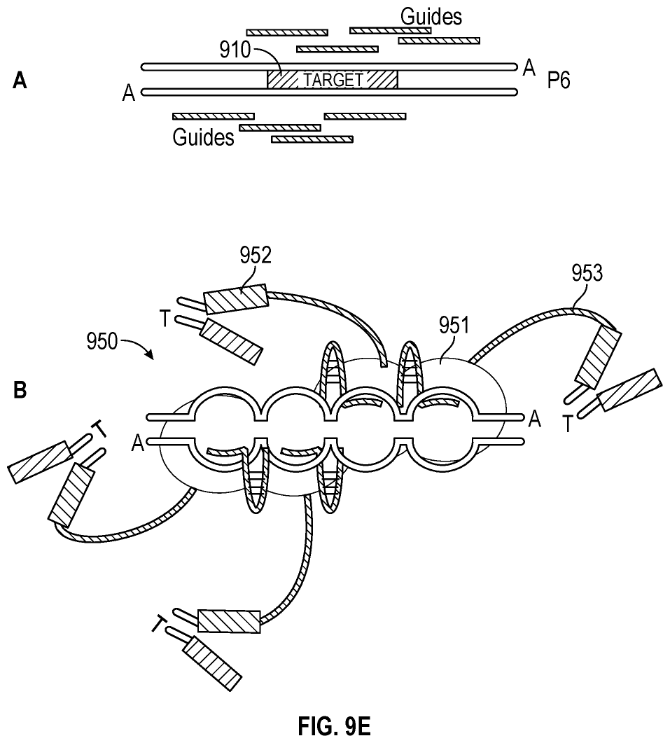

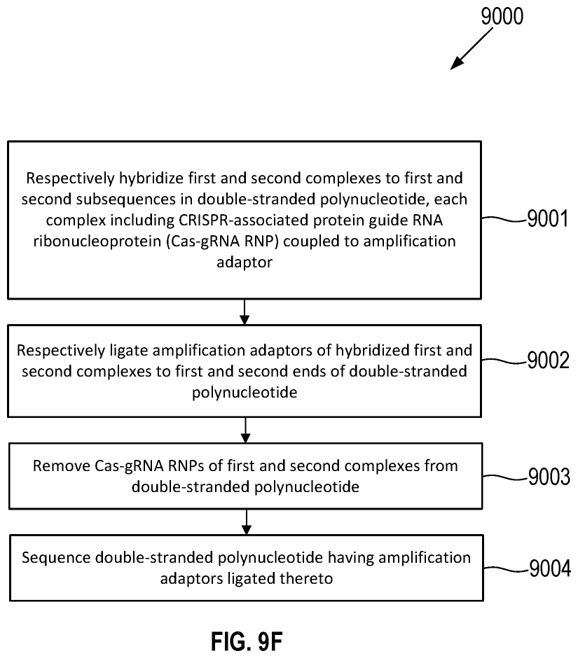

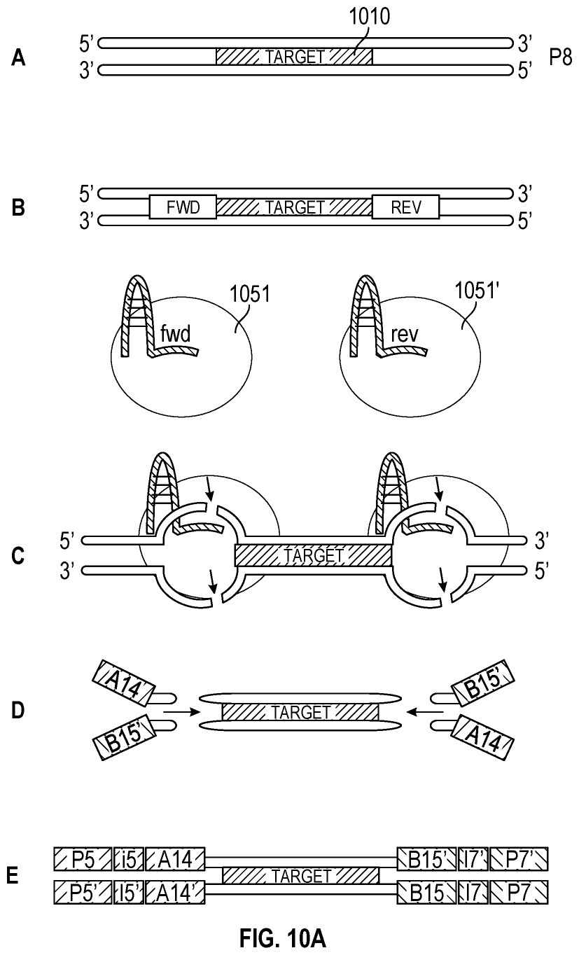

Some methods provided herein solve the problem of long and laborious workflows for targeted sequencing of intact dsDNA fragments. As will be clear from the present disclosure, Cas-gRNA RNPs may provide for rapid and specific hybridization to target regions in polynucleotides, e.g., dsDNA. As now will be described with reference to FIGS. 9A-9F, complexes including Cas-gRNA RNPs and amplification adaptors may be used to ligate amplification adaptors to selected fragments, such that those fragments subsequently may be amplified and sequenced, while other fragments do not become ligated to such adaptors and therefore are not amplified and sequenced. Accordingly, the selected fragments may be enriched and sequenced in a streamlined manner. This may be particularly useful in applications where it may be desirable to preserve and enrich double-stranded polynucleotides during adaptor ligation, e.g., in sequencing cell free DNA (cfDNA), whereas previously known enrichment approaches may involve single stranded polynucleotides. Additionally, or alternatively, it may be useful to label both strands of cfDNA molecules with duplex UMIs for additional accuracy.

While some previously known ligation approaches may be compatible with double-stranded polynucleotides, such approaches may not provide for any enrichment of selected fragments. For example, FIG. 9A schematically illustrates example compositions and operations in a previously known process flow for ligating amplification adaptors to fragments of a dsDNA library. As illustrated at operation A, a dsDNA library may be fragmented. Such fragmentation may occur naturally, e.g., in the case of cfDNA, may be performed mechanically or enzymatically, or may be generated from an RNA library. The resulting plurality of fragments may have uneven ends, which may be blunted using end repair in a manner such as illustrated in operation B of FIG. 9A. The 5′ ends then may be phosphorylated in a manner such as illustrated in operation C of FIG. 9A. Non-templated A nucleotides then are added to the 3′ ends using A-tailing in a manner such as illustrated in operation D of FIG. 9A. Y-shaped (forked) amplification adaptors then may be coupled to the fragments using adaptor ligation in a manner such as illustrated in operation E of FIG. 9A. The adaptors may have sequences that allow for the identification of both originating strands after PCR amplification. As illustrated in operation F of FIG. 9A, the fragments then may be amplified using PCR, during which sample indexes may be added. The amplified fragments then may be sequenced. From the process flow illustrated in FIG. 9A, it will be understood that substantially each dsDNA fragment present in operation A ultimately may have amplification adapters ligated thereto, and thus may be amplified and sequenced. While it may be desirable in some circumstances to obtain the sequences of substantially all of the dsDNA fragments in a given sample, in other circumstances it may be desired to sequence only a small, selected subset of the fragments, e.g., fragments of cfDNA.

In comparison to the previously known process flow described with reference to FIG. 9A, FIGS. 9B-9F schematically illustrate example compositions and operations in a process flow for ligating amplification adaptors to selected polynucleotide fragments using Cas-gRNA RNPs. As illustrated at operation A, a dsDNA library may be fragmented. Such fragmentation may occur naturally, e.g., in the case of cfDNA, may be performed mechanically or enzymatically, or may be generated from an RNA library. Some of the fragments may include respective target sequence(s) that it is desired to enrich and detect, while other fragments may not necessarily include such sequence(s); for example, the fragment P5 illustrated in FIG. 9A includes target sequence 910, while other fragments may include other target sequences or may not include any such target sequences.

In a manner similar to that described with reference to FIG. 9A, the resulting plurality of fragments may have uneven ends, which may be blunted using end repair in a manner such as illustrated in operation B of FIG. 9A. The 5′ ends then may be phosphorylated in a manner such as illustrated in operation C of FIG. 9A. Non-templated A nucleotides then are added to the 3′ ends using A-tailing in a manner such as illustrated in operation D of FIG. 9A. In a manner such as described in greater detail below with reference to FIG. 9C, Y-shaped (forked) amplification adaptors then may be selectively coupled to the fragments which include target sequence 910, while such adaptors are not added to any fragments lacking that sequence, in a manner such as illustrated in operation E of FIG. 9B. The adaptors may have sequences that allow for the identification of both originating strands after PCR amplification. For example, the adaptors may include duplex UMIs. As illustrated in operation F of FIG. 9B, the fragments to which the adaptors were ligated then may be amplified using PCR, during which sample indexes may be added, while the fragments to which adaptors were not ligated are not amplified. The amplified fragments then may be sequenced, while the fragments to which adaptors were not ligated are not sequenced. From the process flow illustrated in FIG. 9A, it will be understood that substantially only the polynucleotide fragments present in operation A that include target sequence 910 ultimately may have amplification adapters ligated thereto, and thus may be amplified and sequenced. Accordingly, the process flow illustrated in FIG. 9B provides a streamlined manner of selectively sequencing a subset of the fragments in a given sample, e.g., fragments of cfDNA.

FIG. 9C schematically illustrates further details regarding the manner in which adaptors may be selectively coupled to the fragments P6 which include target sequence 910. As illustrated in FIG. 9C, at operation A the fragments P6 may be contacted with first and second complexes 950, 950′ respectively including an enzymatically deactivated Cas-gRNA RNP 951 coupled to amplification adaptor(s) 952 via a linker 953. For example, a plurality of the complexes 950, 950′ may be mixed with fragmented, A-tailed sample dsDNAs. The gRNA of each of the Cas-gRNA RNPs 951 may target a specific region (subsequence) within a respective single strand of the dsDNA, and the regions may be staggered so that the Cas-gRNA RNPs hybridize to respective strands at locations that are offset from one another and that are on opposing sides of a double-stranded target region 910 that it is desired to enrich. For example, in a manner such as illustrated in operation A of FIG. 9C, the gRNA of the Cas-gRNA RNP 951 of complex 950 may target a region that is forward (“fwd”) of target sequence 910, and the gRNA of the Cas-gRNA RNP 951 of complex 950′ may target a region that is reverse (“rev”) of target sequence 910. As such, the guide sequences of first and second complexes 950, 950′ may be considered to “flank” target sequence 910 in the forward and reverse directions. It will be appreciated that any suitable number of gRNAs may be designed to direct corresponding Cas-gRNA RNPs of the complexes hybridize at respective strands at locations that flank specific sequences within dsDNA fragments. For example, multiple different gRNAs (e.g., 1000-100,000 gRNAs, or more than 100,000 gRNAs) may be used so as to simultaneously enrich for many different sequences of interest in a sample. Note that the gRNAs need not necessarily “flank” a given target sequence 910, but rather that at least two guides per target sequence may bind to opposing strands within a given fragment P6. The gRNAs, and corresponding complexes, may not bind to any fragments that lack a sequence that such gRNAs target. Note that the use of at least two Cas-gRNA RNPs for each fragment to receive an adaptor at each end is expected to help with specificity.

In some examples, the adaptors 952 of complexes 950, 950′ may be or include Y-shaped adaptor pairs similar to those described with reference to FIG. 3D, FIG. 8C, or FIG. 8D. Optionally, the adaptors may include UMIs in a manner such as described with reference to FIG. 9D. Additionally, or alternatively, the adaptors may include unpaired Ts which may hybridize to any A-tails on the fragment. In this regard, note that the specific binding of the Cas-gRNA RNPs 951 to respective subsequences of the fragment is expected to be relatively rapid and strong, and thus favored over the non-specific binding of T-base adaptor pairing to A-tails of the fragments. This selectivity may be enhanced by hybridizing the Cas-gRNA RNPs 951 to respective subsequences at elevated temperature. Additionally, unwanted background ligation may be reduced by reducing the concentration of the complexes 950, 950′ may be significantly reduced compared to standard ligation conditions. For example, in previously known methods the adaptors normally are in a large excess over the template (e.g., 10-1000× over the template), whereas in the present examples the adaptors 952 may be provided in a significantly lower concentration than the template (e.g., 0.001-0.1× relative to the template) so as to provide a low background as only a sub-portion of the total fragments are being targeted.

From operation A illustrated in FIG. 9C, it will further be appreciated that when the gRNAs of the first and second complexes 950, 950′ hybridize to respective subsequences of a given fragment, the adaptors 952 of those complex are brought into proximity of the ends of that fragment. Accordingly, as illustrated in operation B of FIG. 9C, the amplification adaptor(s) 952 of first complex 950 may be ligated to a first end of fragment P6, and the amplification adaptor(s) 952 of second complex 950′ may be ligated to a second end of fragment P6, using a ligase (not specifically illustrated) with which the complexes and fragments are contacted during operation B. The ligase further may seal the bonds between the adaptors and the ends of the fragments. As one nonlimiting example, the ligase may include a T4 DNA ligase. Following ligation of adaptors 952 to respective ends of fragments P6 that include target sequences that are flanked by subsequences for which the gRNAs of Cas-gRNA RNPs 951 are specific, the Cas-gRNA RNPs 951 may be heat killed and removed, or removed using a suitable reagent such as Proteinase K, SDS, or a protease. Any remaining linkers 953 may remain coupled to adaptors 952, and thus may remain coupled to the fragments including target sequence 910 in a manner such as illustrated in FIG. 9C. The fragment, coupled to adaptor(s) 952, then may be amplified and sequenced in a manner such as described elsewhere herein. Any fragments lacking target sequence 910 may not be coupled to adaptor(s) 952, and thus may not be amplified and sequenced. As such, the fragments including target sequence 910 are enriched.

It will be appreciated that operations illustrated in FIG. 9C may be performed in any suitable order. In some examples, the Cas-gRNA RNPs 951 are hybridized to respective subsequences, thus bringing adaptors 952 into proximity of the ends of the corresponding fragment P6, in a separate operation performed before a ligase is added and used to ligate those adaptors to the ends of that fragment. In other examples, the Cas-gRNA RNPs 951 are hybridized to respective subsequences in the presence of the ligase, such that the ligase may ligate those adaptors to the ends of that fragment relatively quickly. Alternatively, in such examples, ATP may be added after a period of time as a “switch” to separate the ligation operation from the Cas-gRNA RNP hybridization operation, so that the hybridization substantially may be performed (in the presence of inactive ligase) before the ligation is performed (in the presence of ligase which is activated by the newly added ATP).

Additionally, or alternatively, in some examples, fragments including target sequence 910 selectively may be coupled to substrate(s) in a manner similar to that described with reference to FIGS. 8A-8H. For example, any suitable portion of a complex 950, 950′, such as the gRNA, Cas-gRNA RNP 951, or the adaptor 952 may be functionalized and then coupled via such functionalization to a substrate. For example, the complex may be coupled to a tag or tag partner, and the substrate coupled to a tag partner or tag that reacts to couple the complex to the substrate. Any fragments that do not include target sequence 910, and thus do not become coupled to complexes 950, 950′, also do not become coupled to the substrate (e.g., because they lack a tag or tag partner to react with the tag partner or tag at the substrate) and may be washed away. Illustratively, the tag partners may include SNAP proteins and the tags may include O-benzylguanine; the tag partners may include CLIP proteins and the tags may include O-benzylcytosine; the tag partners may include SpyTag and the tags may include SpyCatcher; the tag partners may include SpyCatcher and the tags may include SpyTag; the tag partners may include biotin and the tags may include streptavidin; the tag partners may include streptavidin and the tags may include biotin; the tag partners may include NTA and the tags may include His-Tag; the tag partners may include His-Tag and the tags may include NTA; the tag partners may include antibodies (such as anti-FLAG antibodies) and the tags may include antigens for which the antibodies are selective (such as FLAG tags); the tag partners may include antigens (such as FLAG tags) and the tags may include antibodies that are selective for the antigens (such as anti-FLAG antibodies); or the tag partners may include a first oligonucleotide and the tags may include a second oligonucleotide that is complementary to, and hybridizes to, the first oligonucleotide. The tag partners may be coupled to the substrate via any suitable linkage, e.g., via a covalent linkage or via a non-covalent linkage. Similarly, the tags respectively may be coupled to the complexes 950, 950′ via any suitable linkage, e.g., via a covalent linkage or via a non-covalent linkage.

It will be appreciated that complexes 950, 950′ may be prepared in any suitable manner. As noted above with reference to FIG. 9B, complexes 950, 950′ may include a Cas-gRNA RNP 951 which includes gRNA targeted to a particular subsequence, and which is coupled to adaptor(s) 952 via a linker 953. FIG. 9D schematically illustrates example configurations of complex 950. In both example A and example B illustrated in FIG. 9D, the Cas of the Cas-gRNA 951 may be engineered so as not to cut the target polynucleotide at the sequence to which the gRNA is complementary, e.g., may include dCas9. In both example A and example B illustrated in FIG. 9D, Y-shaped amplification adaptor 952 may include read 1 (A14) and read 2 (B15) adaptors and ME/ME′ regions in a manner similar to that described with reference to FIGS. 8A-8H. Optionally, adaptor 952 may include an unpaired T to hybridize to the A-tail of the fragment. Alternatively, adaptor 952 may be ligated to a blunt end. Additionally, or alternatively, adaptor 952 may include a double stranded duplex UMI as illustrated in FIG. 9D. In example A illustrated in FIG. 9D, adaptor 952 is conjugated to the Cas protein of Cas-gRNA RNP 951 via linker 953, e.g., a protein-based linker. For example, the Cas protein and tether 953 may be co-expressed, or suitably coupled to one another after expression in a manner such as described elsewhere herein, or in a manner such as described in Aird et al., “Increasing Cas-9 mediated homology-directed repair efficiency through covalent tethering of DNA repair template,” Communications Biology 1, 54 (2018), doi.org/10.1038/s42003-018-0054-2. In example B illustrated in FIG. 9D, adaptor 952 is coupled to the gRNA of Cas-gRNA RNP 951 via linker 953, e.g., an oligonucleotide-based linker in a manner such as described elsewhere herein. However, it will be appreciated that linker 953 may include any suitable protein, polynucleotide, or polymer (e.g., PEG).

It will further be appreciated that plurality of different subsequences may be used to enrich for fragments including a desired target sequence 910. For example, operation A of FIG. 9E illustrates how multiple gRNAs (“guides”) may be designed that tile over and around a target sequence 910 of fragment P6. Upon binding to respective subsequences in the fragment, complexes 950 including such gRNA may saturate that fragment over some or all of the target sequence 910 in a manner such as illustrated in operation B of FIG. 9E. This strategy may help to enrich fragments that are randomly fragmented and/or may include breaks within target sequence 910, by increasing the likelihood of coupling complexes 950 to that sequence and thus placing respective adaptors 952 in sufficient proximity to the ends of that fragment for ligation to those ends, such that the fragment subsequently may be amplified and sequenced. For example, based on the length of linker 953, the adaptor 952 may be ligated to a fragment end that is within a defined number of base pairs of the subsequence to which the Cas-gRNA RNP of the respective complex is coupled, e.g., about 5-30 base pairs, or about 10-25 base pairs, or about 15-20 base pairs.

FIG. 9F illustrates a flow of operations in an example method 9000 of generating a fragment of a double-stranded polynucleotide. Method 9000 illustrated in FIG. 9F may include respectively hybridizing first and second complexes to first and second subsequences in the double-stranded polynucleotide (operation 9001). Each of the first and second complexes may include a CRISPR-associated protein guide RNA ribonucleoprotein (Cas-gRNA RNP) coupled to an amplification adaptor. For example, in a manner such as described with reference to operation A of FIG. 9C, complexes 950, 950′ respectively may include a Cas-gRNA RNP 951 coupled to an amplification adaptor 952. In nonlimiting examples, the Cas-gRNA RNP may include dCas9.

Optionally, each complex further may include a linker 953 coupling the Cas-gRNA RNP to the amplification adapter. In some examples, the complexes may be prepared in a manner such as described with reference to FIG. 9D. For example, the linker may be coupled to the Cas of the Cas-gRNA RNP. Or, for example, the linker may be coupled to the gRNA. In some examples, the linker may include a protein, a polynucleotide, or a polymer. In some examples, the amplification adaptors are Y-shaped. Additionally, or alternatively, the amplification adaptors respectively may include unique molecular identifiers. Additionally, or alternatively, method 9000 further may include A-tailing the double-stranded polynucleotide prior to the hybridizing, and the amplification adaptor comprises an unpaired T to hybridize with the A-tail. Alternatively, the amplification adaptor may be ligated to a blunt end.

The gRNAs of complexes 950, 950′ may be selected so as to hybridize to subsequences on respective strands of double-stranded polynucleotide P6, e.g., to flank target sequence 910, at locations that are sufficiently near to respective ends of the polynucleotide that the amplification adaptors may become ligated to such ends. In some examples, the first subsequence is 3′ of a target sequence along a first strand of the double-stranded polynucleotide, and the second subsequence is 3′ of the target sequence along a second strand of the double-stranded polynucleotide.

Method 9000 illustrated in FIG. 9F further may include respectively ligating the amplification adaptors of the hybridized first and second complexes to first and second ends of the double-stranded polynucleotide (operation 9002). For example, in a manner such as described with reference to operation B of FIG. 9C, hybridization of complexes 950, 950′ to respective subsequences brings the corresponding amplification adaptors 952 into sufficient proximity to respective ends of the polynucleotide to become ligated thereto. The ligating may include using a ligase. In a manner such as described with reference to operation B of FIG. 9C, the ligase optionally may be present during the hybridizing. The ligase may be inactive during the hybridizing and may be activated for the ligating using ATP. Alternatively, the ligase may be added after the hybridizing.

Method 9000 illustrated in FIG. 9F further may include removing the Cas-gRNA RNPs of the first and second complexes from the double-stranded polynucleotide (operation 9003), for example in a manner such as described with reference to operation C of FIG. 9C. In examples in which the complexes include linkers 953, the linker optionally may remain coupled to the amplification adaptor when the Cas-gRNA RNP is removed, e.g., in a manner such as described with reference to FIG. 9C.

Method 9000 illustrated in FIG. 9F may include sequencing the double-stranded polynucleotide having the amplification adaptors ligated thereto (operation 9004), e.g., in a manner such as described elsewhere herein.

Generating Fragments with 5′ Overhangs, and Coupling Adaptors Thereto

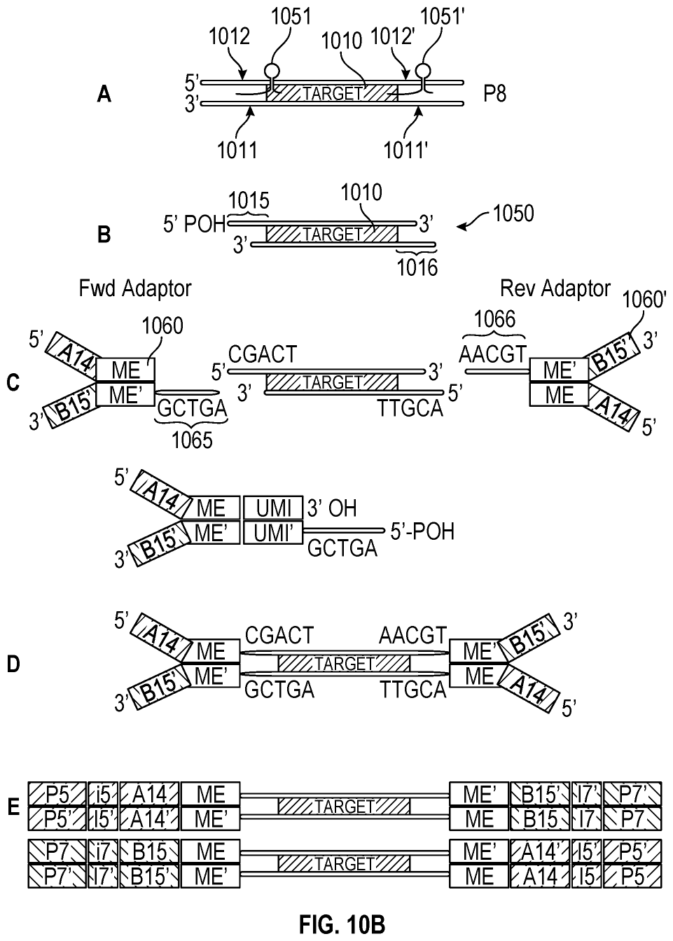

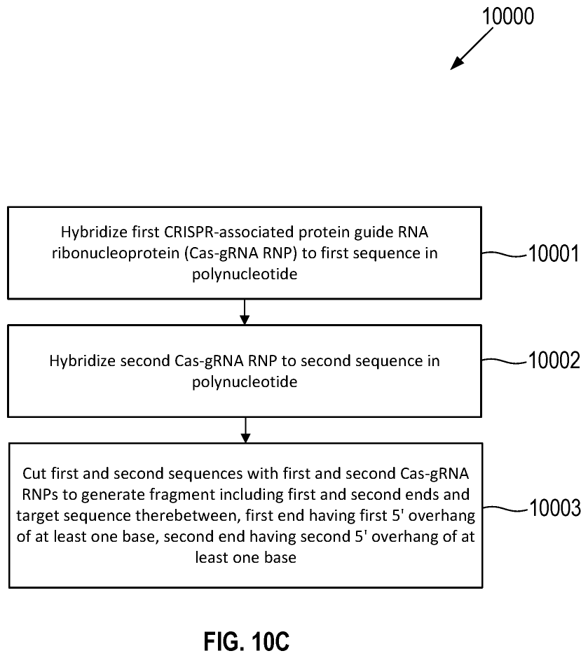

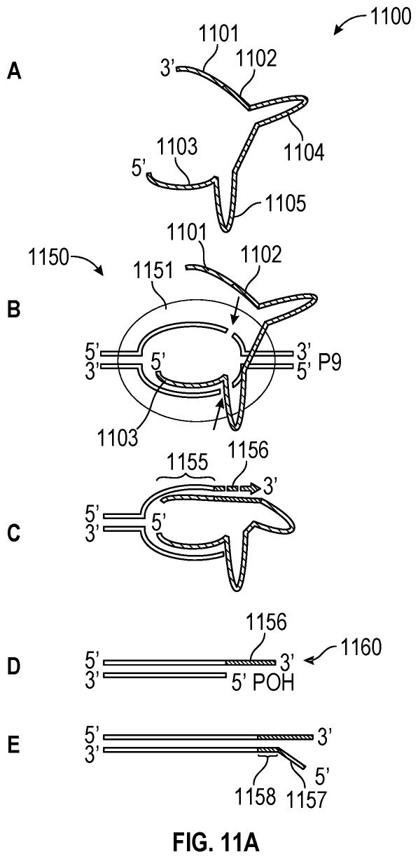

In some examples, methods and compositions provided herein solve the problem of long and laborious workflows for targeted amplification and/or targeted sequencing. As will be apparent from the present disclosure, Cas-gRNA RNPs may be used to generate fragments of polynucleotides as part of a target enrichment method. Amplification adaptors may be added using a number of additional steps, e.g., using end repair, A-tailing, and adaptor ligation in a manner such as described elsewhere herein. As will now be described with reference to FIGS. 10A-10C, Cas-gRNA RNPs may be used to generate fragments with 5′ overhangs to which amplification adaptors, also having 5′ overhangs, readily may be ligated with relatively few and simple steps. As provided herein, the combination of rapid Cas-gRNA RNP based enrichment by fragmentation and streamlined adaptor addition provide for faster and easier complete workflows for targeted sequencing applications. In particular, certain types of Cas-gRNA RNPs may be used to generate fragments that are ready for adaptor ligation, without the need for end repair or A-tailing.

FIGS. 10A-10C schematically illustrate example compositions and operations in a process flow for generating fragments using Cas-gRNA RNPs and coupling adaptors thereto. Referring first to FIG. 10A, at operation A polynucleotide P8 may include target sequence 1010 that it is desired to enrich, amplify, and sequence. Illustratively, target sequence 1010 may be about 150-600 base pairs long, or any other length such as exemplified herein. In a manner similar to that provided elsewhere herein, at operation B polynucleotide P8 may be contacted with first and second Cas-gRNA RNPs 1051, 1051′ with guide RNA sequences that specifically hybridize to first (“fwd”) and second (“rev”) sequences in polynucleotide P8 that flank target sequence 1010. First and second Cas-gRNA RNPs 1051, 1051′ respectively may be for cutting the first and second sequences of the polynucleotide to generate a fragment having first and second ends with the target sequence therebetween. For example, as illustrated at operation C of FIG. 10A, first Cas-gRNA RNP 1051 may hybridize to first sequence (“fwd”) in polynucleotide P8, and second Cas-gRNA RNP 1051′ may hybridize to second sequence (“rev”) in the polynucleotide. In a manner such as described elsewhere herein, the first and second Cas-gRNA RNPs 1051, 1051′ may cut polynucleotide P8 at locations that flank target sequence 1010 generating a fragment including target sequence 1010. Optionally, in a manner such as will be described with reference to FIG. 10B, the first end of the fragment generated in operation C optionally may have a first 5′ overhang of at least one base, and the second end of the fragment optionally may have a second 5′ overhang of at least one base. That is, particular types of Cas-gRNA RNPs optionally may be used that generate such overhangs, e.g., such as described with reference to FIG. 10B.

As illustrated in operation D of FIG. 10A, and in a manner as will be described below with reference to FIG. 10B, amplification adaptors (e.g., A14 and B15 sequences in a Y-shaped adaptor) may be ligated to the first and second ends of the fragment. Optionally, in a manner such as described with reference to FIG. 10B, a first amplification adaptor optionally may have a 5′ overhang that is complementary to a 5′ overhang at a first end of the fragment, and a second amplification adaptor may have a 5′ overhang that is complementary to a 5′ overhang at a second end of the fragment. As illustrated in operation E of FIG. 10A, the fragment having adaptors coupled thereto may be amplified (e.g., using PCR) so as to add a sample index (i7 and the complement thereof) and sequencing adaptors (e.g., P5 and P7 adaptors and the complements thereof). During the amplification, each fragment produces bi-directional amplicons, for use in bi-directional sequencing reads, as the “top” and “bottom” strands of the targeted region generate different orientations due to the ligation of the forked adaptor structure. This means that the two sequencing reads may be performed from either end of the target sequence 1010, providing additional coverage. Amplification also adds additional clustering sequences (e.g., P5, P7) and sample index sequences (e.g., i5, i7) for use in multiplexed sequencing. The adaptor sequences shown in FIGS. 10A-10B (e.g., A14, B15, ME) are examples that may be used for Illumina sequencing but may be switched for any other suitable sequence as desired. The resulting enriched fragment, having amplification and sequencing adaptors coupled thereto, then may be sequenced to identify target sequence 1010.

Although a single polynucleotide P8 and corresponding first and second Cas-gRNA RNPs 1051, 1051′ are illustrated in FIG. 10A, it will be appreciated that this approach readily may be scaled in a manner such as provided elsewhere herein, e.g., by contacting a plurality of different polynucleotides with first and second pluralities of Cas-gRNA RNPs with respective guide RNA sequences that specifically hybridize to first or second sequences in selected ones of the polynucleotides that flank target sequences with those polynucleotides.

FIG. 10B schematically illustrates example compositions and operations in a process flow for generating fragments with 5′ overhangs using Cas-gRNA RNPs and coupling adaptors thereto. In the composition illustrated at operation A of FIG. 10B, first Cas-gRNA RNP 1051 is hybridized to a first sequence in polynucleotide P8, and second Cas-gRNA RNP 1051′ is hybridized to a second sequence in the polynucleotide that is spaced apart from the first sequence by at least target sequence 1010. First Cas-gRNA RNP may be configured, and used, to cleave polynucleotide P8 on the first strand at site 1011, and on the second strand at site 1012 which is offset from site 1011 in the 5′ direction by at least one base, e.g., by 2-5 bases, or about 5 bases. Similarly, second Cas-gRNA 1051′ may be configured, and used, to cleave polynucleotide P8 on the first strand at site 1011′, and on the second strand at site 1012′ which is offset from site 1011′ in the 5′ direction by at least one base, e.g., by 2-5 bases, or by about 5 bases. Cas-gRNA RNPs 1051, 1051′ may include any suitable Cas-gRNA RNP may be used that leaves a single-stranded 5′ overhang region of at least one base following dsDNA cleavage. Illustratively, the Cas may include Cas12a, e.g., Cas12a (Cpf1 or C2c1) or FnCas12a, or a Cas12a ortholog such as described in Teng et al., “Enhanced mammalian genome editing by new Cas12a orthologs with optimized crRNA scaffolds,” Genome Biology 20: 15 (2019), the entire contents of which are incorporated by reference herein.

In the composition illustrated at operation B of FIG. 10B, the first end of fragment 1050 generated by operation A may have a first 5′ overhang 1015 of at least one base, and the second end of the fragment may have a second 5′ overhang 1016 of at least one base. For example, the first and second 5′ overhangs each may be about 2-5 bases in length, illustratively about 5 bases in length. The overhangs may be, but need not necessarily be, the same length as one another. At the first end of the fragment, the strand including overhang 1015 may include a 5′ phosphate group, and the other strand may include a 3′ OH group. Similarly, at the second end of the fragment, the strand including overhang 1016 may include a 5′ phosphate group, and the other strand may include a 3′ OH group. First and second 5′ overhangs 1015, 1016 may have different sequences than one another, e.g., as a result of the particular sequences within polynucleotide P8 to which the gRNAs of first and second Cas-gRNA RNPs 1051, 1051′ respectively hybridize.

In the composition illustrated at operation C of FIG. 10B, fragment 1050 is contacted with adaptors 1060, 1060′ that include respective 5′ overhangs 1065, 1066 that respectively are complementary to 5′ overhangs 1015, 1016. The 5′ overhangs 1065, 1066 may have the same length as one another, or may have different lengths than one another. In the nonlimiting example shown in FIG. 10B, 5′ overhang 1065 of “fwd” adaptor 1060 may include, or may consist essentially of, a plurality of bases that are complementary to a plurality of bases in 5′ overhang 1015 of fragment 1050. 5′ overhang 1065 may have the same length as 5′ overhang 1015, e.g., may be about 2-5 bases long, e.g., may be about 5 bases long. 5′ overhang 1066 of “rev” adaptor 1060 may include, or may consist essentially of, a plurality of bases that are complementary to a plurality of bases 5′ overhang 1016 of fragment 1050. 5′ overhang 1066 may have the same length as 5′ overhang 1016, e.g., may be about 2-5 bases long, e.g., may be about 5 bases long. Adaptors 1060, 1060′ may include any other suitable sequences, e.g., such as described elsewhere herein. For example, each adaptor 1060, 1060′ may include a Y-shaped adaptor pair with an optional UMI. In the nonlimiting example illustrated in FIG. 10B, adaptors 1060, 1060′ include forward amplification adaptors (e.g., A14, A14′), reverse amplification adaptors (e.g., B15, B15′), and optionally may include ME/ME′ sequences and/or UMI/UMI′ sequences.

Because first and second 5′ overhangs 1015, 1016 of fragment 1050 may have different sequences than one another, overhangs 1065, 1066 of adaptors 1060, 1060′ similarly may have sequences that are different than one another and that are complementary to a respective fragment overhang 1015, 1016. For example, amplification adaptor 1060 may have a 5′ overhang 1065 that is complementary to the first 5′ overhang 1015 and is not complementary to the second 5′ overhang 1016; and amplification adaptor 1060′ may have a 5′ overhang that is complementary to the second 5′ overhang 1016 and is not complementary to the first 5′ overhang 1015. As such, amplification adaptor 1060 may hybridize with specificity to 5′ overhang 1015, and amplification adaptor 1060′ may hybridize with specificity to 5′ overhang 1016. Illustratively, 5′ overhang 1015 may include the 5-base sequence CGACT to which the 5-base sequence GCTGA of 5′ overhang 1065 may hybridize, and 5′ overhang 1016 may include the 5-base sequence TTGCA to which the 5-base sequence AACGT of overhang 1066 may hybridize. It will be appreciated that these 5-base sequences are intended to be purely illustrative.

Adaptors 1060, 1060′ may be ligated to fragment 1050 in any suitable manner to form a fragment having adaptors coupled thereto such as illustrated in operation D of FIG. 10B. For example, the composition illustrated at operation C of FIG. 10B may include at least one ligase for ligating the first amplification adaptor 1060 to the first end of fragment 1050 and for ligating the second amplification adaptor 1060′ to the second end of fragment 1050. In one nonlimiting example, the ligase may include T4 DNA ligase, although it will be appreciated that other suitable ligases may be used. Following such ligation, as illustrated in operation E of FIG. 10B, the fragment having adaptors coupled thereto may be amplified (e.g., using PCR) so as to add a sample index (i7 and the complement thereof) and sequencing adaptors (e.g., P5 and P7 adaptors and the complements thereof). The resulting enriched fragment, having amplification and sequencing adaptors coupled thereto, then may be sequenced to identify target sequence 1010.

Although a single polynucleotide P8, corresponding first and second Cas-gRNA RNPs 1051, 1051′, and corresponding adaptors 1060, 1060′ are illustrated in FIG. 10B, it will be appreciated that this approach readily may be scaled in a manner such as provided elsewhere herein. For example, operation A described with reference to FIG. 10B may be used to generate a plurality of polynucleotide fragments. As illustrated in operation B of FIG. 10B, each of the fragments may have first and second ends with the target sequence therebetween, the first end having a first 5′ overhang of at least one base, the second end having a second 5′ overhang of at least one base. The first and second 5′ overhangs may have different sequences than one another and than the first and second 5′ overhangs of other fragments. The plurality of fragments may be contacted with a plurality of first amplification adaptors and a plurality of second amplification adaptors in a manner such as described with reference to operation C of FIG. 10B. Each of the first amplification adaptors may have a third 5′ overhang that is complementary to the first 5′ overhang of a corresponding fragment and is not complementary to the second 5′ overhang of that fragment and is not complementary to the first or second 5′ overhangs of other fragments. Each of the second amplification adaptors may have a fourth 5′ overhang that is complementary to the second 5′ overhang of a corresponding fragment and is not complementary to the first 5′ overhang of that fragment and is not complementary to the first or second 5′ overhangs of other fragments. Use of the terms “third” or “fourth” 5′ overhangs, with reference to an amplification adaptor, is intended to assist in distinguish these respective overhangs from the first and second overhangs of the fragments, rather than to suggest that any of the amplification adaptors have three or four 5′ overhangs. Ligases further may be used for ligating the first amplification adaptors to the first ends for which the first and third 5′ overhangs are complementary and for ligating the second amplification adaptors to the second ends for which the second and fourth 5′ overhangs are complementary, e.g., in a manner such as described with reference to operation D of FIG. 10B.

FIG. 10C illustrates a flow of operations in an example method 10000 of generating a fragment of a polynucleotide. Method 10000 may include hybridizing a first CRISPR-associated protein guide RNA ribonucleoprotein (Cas-gRNA RNP) to a first sequence in the polynucleotide (operation 10001), and may include hybridizing a second Cas-gRNA RNP to a second sequence in the polynucleotide that is spaced apart from the first sequence by at least a target sequence (operation 10002). For example, in a manner such as described with reference to operation C of FIG. 10A and operation A of FIG. 10B, the first and second Cas-gRNA RNPs may be selected so as to flank target sequence 1010. Note that operations 10001 and 10002 may be performed at the same time as one another. Method 10000 also may include cutting the first and second sequences with the first and second Cas-gRNA RNPs to generate a fragment comprising first and second ends and the target sequence therebetween, the first end having a first 5′ overhang of at least one base, the second end having a second 5′ overhang of at least one base (operation 10003). For example, the Cas may include Cas12a. In a manner such as described with reference to FIG. 10B, a first amplification adapter with a complementary 5′ overhang may be ligated to the first end of the fragment and a second amplification adapter with a complementary 5′ overhang may be ligated to the second end of the fragment.

Accordingly, it will be appreciated that target sequences within any suitable number of polynucleotides may be enriched through a process in which Cas-gRNA RNPs are used to flank the target sequences of interest with specificity and to generate fragments with 5′ overhangs, and then amplification adaptors with complementary 5′ overhangs are coupled with specificity to the fragments' overhangs so that the fragments selectively may be amplified. The two layers of specificity (via the Cas-gRNA RNPs and via the complementary 5′ overhang ligation on the amplification adaptors) may provide a particularly high level of enrichment, which may be useful when sequencing the resulting fragments.

Generating Fragments with 3′ Overhangs Including Adaptors and Polymerase Extension

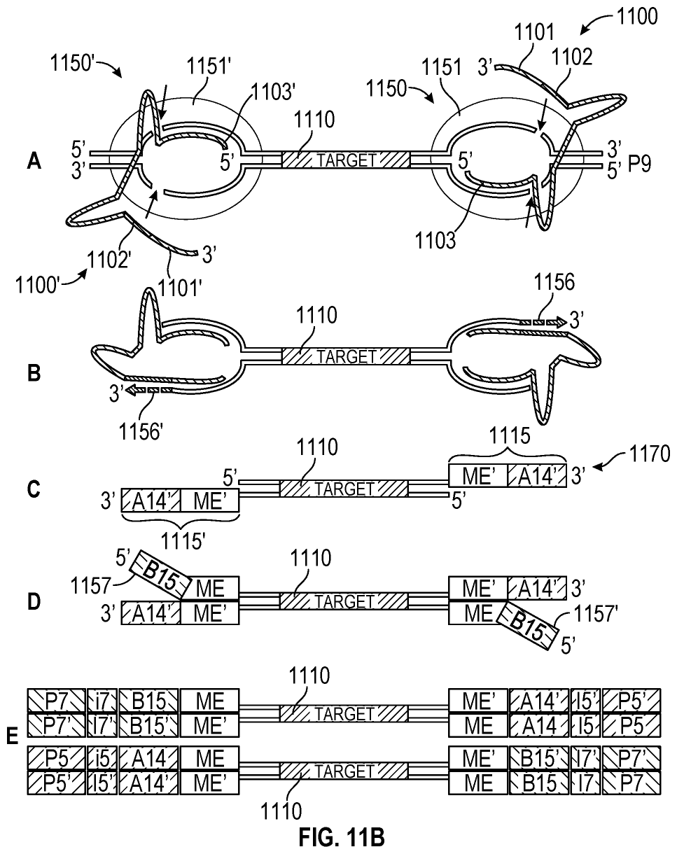

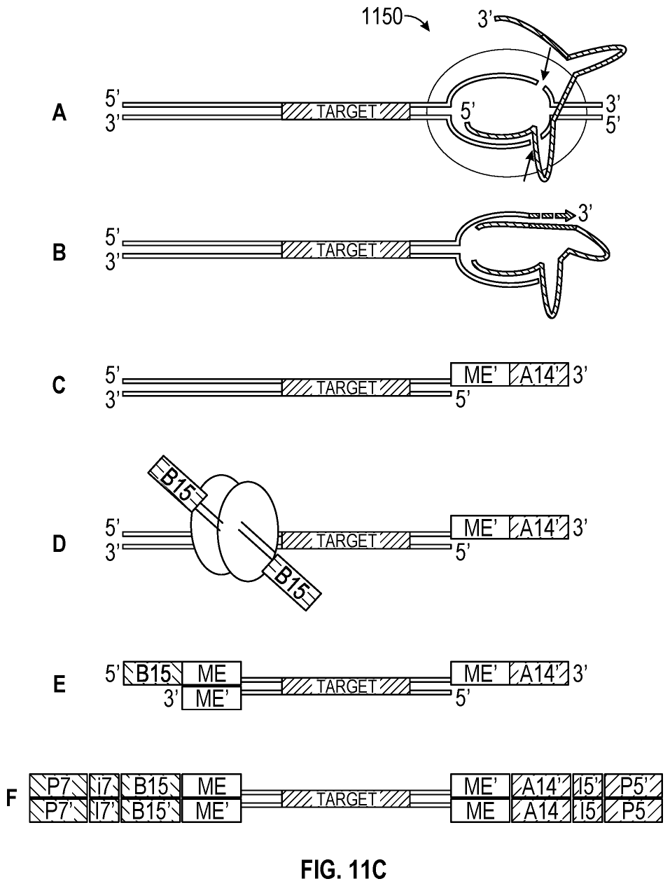

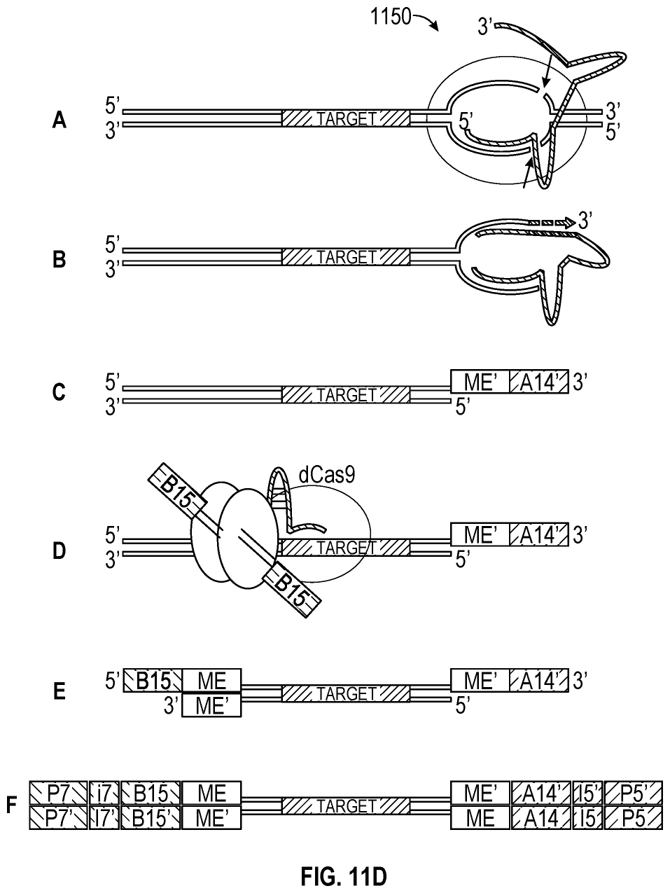

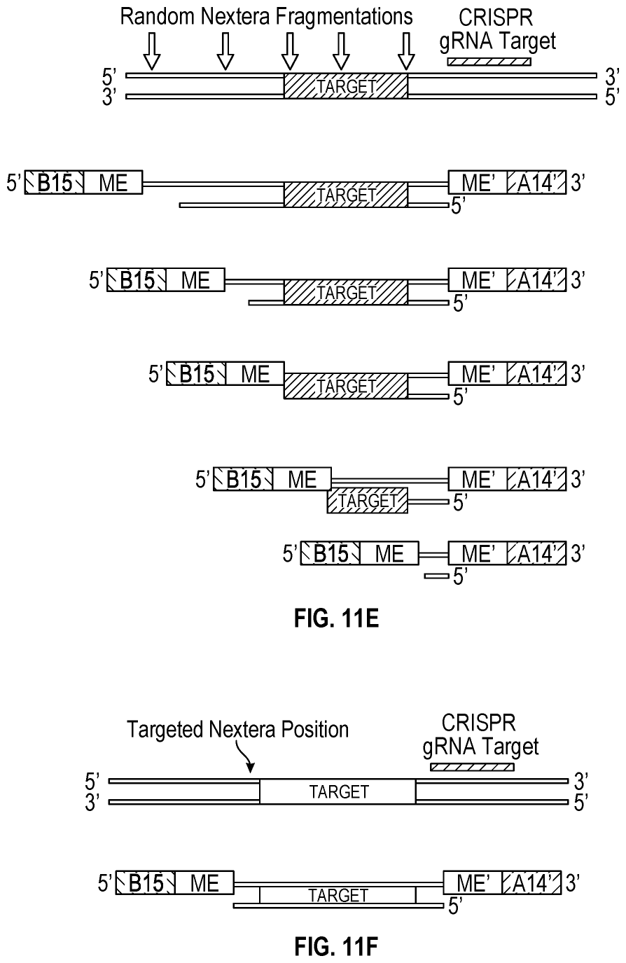

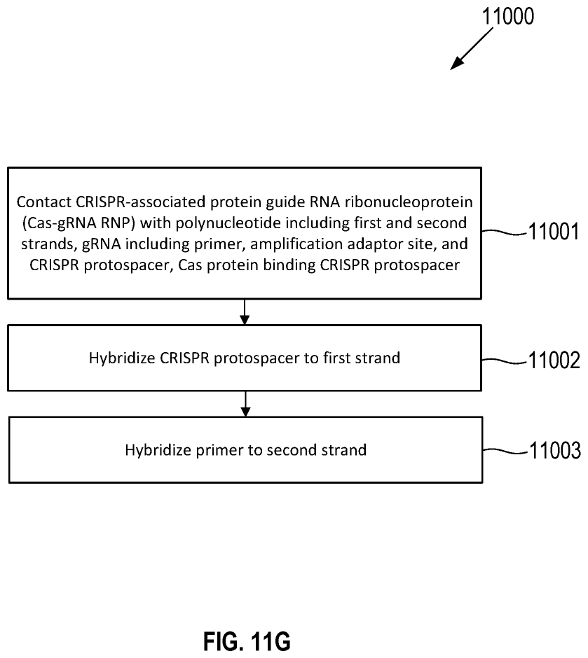

In some examples, methods and compositions provided herein solve the problem of long and laborious workflows for targeted amplification and/or targeted sequencing. As will be apparent from the present disclosure, Cas-gRNA RNPs may be used to generate fragments of polynucleotides as part of a target enrichment method. Amplification adaptors may be added using a number of additional steps, e.g., using end repair, A-tailing, and adaptor ligation in a manner such as described elsewhere herein. As will now be described with reference to FIGS. 11A-11G, Cas-gRNA RNPs including modified gRNA, which gRNA includes primer binding sites and amplification adaptor sites, may be used to generate fragments with 3′ overhangs that include amplification adaptors. As provided herein, the combination of rapid Cas-gRNA RNP based enrichment by fragmentation and streamlined adaptor addition provide for faster and easier complete workflows for targeted sequencing applications. In particular, the Cas-gRNA RNPs may be used to generate fragments that include at least a subset of the adaptors needed for amplification, without the need for end repair, A-tailing, or ligating a full set of adaptors.

FIGS. 11A-11G schematically illustrate example compositions and operations in a process flow for generating fragments using Cas-gRNA RNPs and coupling adaptors thereto. Referring first to FIG. 11A, at operation A at least one gRNA 1100 is provided that includes primer binding site 1101, amplification adaptor site 1102, and CRISPR protospacer 1103. In the nonlimiting example illustrated in FIG. 11A, amplification adaptor site 1102 is located between primer 1101 and CRISPR protospacer 1103. Primer binding site 1101 may be approximately complementary to at least a portion of CRISPR protospacer 1103, e.g., such that the primer binding site and CRISPR protospacer may hybridize to complementary strands of a polynucleotide in a manner such as described in greater detail herein. The gRNA optionally may include loops 1104 and/or 1105 which may be located between amplification adaptor site 1102 and CRISPR protospacer 1103. For further details regarding extended gRNA that includes loops and CRISPR protospacers, see Anzalone et al., “Search-and-replace genome editing without double-strand breaks or donor DNA,” Nature 576: 149-157 (2019), the entire contents of which are incorporated by reference herein.

As illustrated in operation B of FIG. 11A, CRISPR protospacer 1103 of the gRNA of operation A may be bound by the Cas protein 1151 of a first Cas-gRNA RNP 1150. In a manner such as illustrated in operation B of FIG. 11A, primer binding site 1101 and amplification adaptor site 1102 may extend outside of the Cas protein. Cas protein 1151 may be configured to perform double-stranded polynucleotide cleavage, e.g., may include Cas9, Cas12a, or Cas12f. Cas-gRNA RNP 1150 may form a complex with polynucleotide P9, wherein first CRISPR protospacer 1103 is hybridized to a first strand of polynucleotide P9 and first primer binding site 1101 is hybridized to the second strand of the polynucleotide. The first and second strands may be cut by the first Cas-gRNA RNP at respective locations based upon the sequence of the first CRISPR protospacer 1103. Such cutting may, for example, be performed following the hybridization at least of first CRISPR protospacer 1103 to the first strand of polynucleotide P9. In some examples, subsequent to such cutting, then first primer binding site 1101 hybridizes to the second strand of polynucleotide P9.

Note that the gRNA 1100 of Cas-gRNA RNP 1150 includes 3′ extension that is relatively long as compared to gRNA that may be used in certain other examples herein, and includes primer binding site 1101 and adaptor site 1102 that may be used to attach an amplification adaptor to the cut 3′ end of the second polynucleotide strand. More specifically, as illustrated in operation C of FIG. 11A, when primer binding site 1101 hybridizes to portion 1155 of the second strand, near the 3′ end which was cut by Cas 1151, adaptor site 1102 is positioned at a location which is 3′ of the duplex between primer binding site 1101 and portion 1155. A polymerase (such as a reverse transcriptase (RT)) may be included in operation C that uses portion 1155 of the duplex as a primer from which to extend the 3′ end based on the sequence of adaptor site 1102. The polymerase thus may generate an amplicon 1156 of adaptor site 1102 at the cut in the second strand which was caused by Cas protein 1151, and the amplicon may be used as an amplification adaptor. The polymerase (e.g., RT) optionally may be coupled to Cas protein 1151, e.g., in a manner similar to that described in Anzalone et al. For example, the RT and Cas protein 1151 may be components of a first fusion protein or otherwise suitably coupled to one another. Alternatively, the RT may be added during any suitable operation, e.g., during operation B or operation C illustrated in FIG. 11A.

Following double-stranded cleaving of polynucleotide P9 at operation B and generation of amplification adaptor 1156 at operation C, the RT and Cas protein 1151 may be dissociated from polynucleotide P9, e.g., using heat, or any other method (e.g., use of a reagent such as Proteinase K, protease, or SDS) yielding fragment 1160 illustrated in operation D of FIG. 11A. Fragment 1160 may include a 3′ overhang which includes, or consists essentially of, amplification adaptor 1156. A 5′ amplification adaptor 1157 then may be coupled to the cut 5′ end of fragment 1160, opposite adaptor 1156. For example, amplification adaptor 1157 may include a subsequence 1158 that is complementary to, and thus hybridizes to, a corresponding subsequence of adaptor 1156. The hybridized amplification adaptor 1157 may be sealed with a DNA ligase to the cut 5′ end of fragment 1160, forming anew 5′ end.

While FIG. 11A details the manner in which a polynucleotide may be cut at a first region and amplification adaptors added to the resulting cut end, it should be appreciated that the polynucleotide also may be cut at a second region and amplification adaptors added to the resulting cut end. That is, the set of cuts may be used to form a fragment which is suitable for amplification and sequencing. The fragment may include a target sequence, and the cutting and amplification steps may enrich for the target sequence in a manner similar to that described elsewhere herein.

For example, as illustrated in operation A of FIG. 11B, polynucleotide P9 may be contacted with a first Cas-gRNA RNP 1150 configured similarly as described with reference to FIG. 11A, and a second Cas-gRNA RNP 1150′ including second gRNA 1100′. The second gRNA 1100′ may include a second primer binding site 1101′, a second amplification adaptor site 1102′, and a second CRISPR protospacer 1103′ which are configured similarly as described for guide RNA 1100. In a manner similar to that described elsewhere herein, the first and second CRISPR protospacers 1103, 1103′ may target sequences that flank target sequence 1110. As illustrated in FIG. 11B, the second CRISPR protospacer 1103′ may be hybridized to the first strand (that is, the strand opposite that to which the first CRISPR protospacer 1103 hybridizes), and the second primer binding site 1101′ is hybridized to the second strand (that is, the strand opposite to that which the primer binding site 1101 hybridizes). In a manner similar to that described with reference to FIG. 11A, second Cas protein 1151′ binds the second CRISPR protospacer 1103′, and optionally may include Cas9 or other suitable Cas protein that may generate cuts in double-stranded polynucleotides.

In a manner similar to that described with reference to FIG. 11A, the first and second strands of polynucleotide P9 may be cut by the first Cas-gRNA RNP 1150 at respective locations based upon the sequence of first CRISPR protospacer 1103, and also may be cut by the second Cas-gRNA RNP 1150′ at respective locations based upon the sequence of the second CRISPR protospacer 1103′. As may be understood from operation A of FIG. 11B, the cuts in the first and second strands by the second Cas-gRNA RNP are spaced apart from the cuts in the first and second strands by the first Cas-gRNA RNP by at least target sequence 1110. In operation B of FIG. 11B, in a manner such as described with reference to operation C of FIG. 11A, a first polymerase (e.g., RT) may be provided for creating an amplicon of the amplification adaptor site 1102 at the cut in the first strand caused by the first Cas protein 1151, and a second polymerase (e.g., RT) may be provided for creating an amplicon of the amplification adaptor site 1102′ at the cut in the second strand caused by the second Cas protein. In some examples, the second polymerase (e.g., RT) may be coupled to the second Cas protein; for example, the second polymerase and second Cas protein 1151′ optionally may be components of a second fusion protein.

At operation C illustrated in FIG. 11B, the Cas-gRNA RNPs 1150, 1150′ and polymerases may be removed to yield a partially double-stranded polynucleotide fragment 1170 that includes first and second ends, and target sequence 1110 located between the first and second ends. The first end may include a first 3′ overhang 1115, which may include a first amplification adaptor 1156 (e.g., A14′ and optional ME′ sequence or other suitable sequence which was included in first adaptor site 1102). The second end may include a second 3′ overhang 1115′, which may include second amplification adaptor 1156′ (e.g., A14′ and optional ME′ sequence or other suitable sequence which was included in the second adaptor site). As illustrated in operation D of FIG. 11B, a 5′ amplification adaptor 1157 then may be coupled to the cut 5′ end of fragment 1170, opposite adaptor 1156. For example, amplification adaptor 1157 may include an ME (or other) sequence that is complementary to, and thus hybridizes to, a corresponding ME′ (or other) sequence of adaptor 1156. Similarly, amplification adaptor 1157′ may include an ME (or other) sequence that is complementary to, and thus hybridizes to, a corresponding ME′ (or other) sequence of adaptor 1156′. The hybridized amplification adaptors 1157, 1157′ may be sealed with a DNA ligase to the cut 5′ end of fragment 1160, forming a new 5′ end.

As illustrated in operation E of FIG. 11B, the fragment having adaptors 1156, 1157, 1156′, 1157′ coupled thereto may be amplified (e.g., using PCR) so as to add sample indexes (i5 and i7 and the complements thereof) and sequencing adaptors (e.g., P5 and P7 adaptors and the complements thereof). During the amplification, each fragment produces bi-directional amplicons, for use in bi-directional sequencing reads, as the “top” and “bottom” strands of the targeted region generate different orientations due to the ligation of the forked adaptor structure. This means that the two sequencing reads may be performed from either end of the target sequence 1110, providing additional coverage. Amplification also adds additional clustering sequences (e.g., P5, P7) and sample index sequences (e.g., i5, i7) for use in multiplexed sequencing. The adaptor sequences shown in FIG. 11B (e.g., A14, B15, ME) are examples that may be used for Illumina sequencing but may be switched for any other suitable sequence as desired. The resulting enriched fragment, having amplification and sequencing adaptors coupled thereto, then may be sequenced to identify target sequence 1110.

Although a single polynucleotide P9 and corresponding first and second Cas-gRNA RNPs 1150, 1150′ are illustrated in FIGS. 11A-11B, it will be appreciated that this approach readily may be scaled in a manner such as provided elsewhere herein, e.g., by contacting a plurality of different polynucleotides with first and second pluralities of Cas-gRNA RNPs with respective guide RNA sequences (particularly CRISPR protospacers) that specifically hybridize to first or second sequences in selected ones of the polynucleotides that flank target sequences with those polynucleotides.

It will be appreciated that FIG. 11B illustrates a nonlimiting example of a process flow for adding amplification adaptors to both ends of a fragment being enriched, and that other process flows suitably may be used. FIG. 11C illustrates an example including operation A in which Cas-gRNA RNP 1150 is used to generate cuts in polynucleotide P10 in a manner such as described with reference to operations A and B of FIG. 11A and operation A of FIG. 11B. In operation B of FIG. 11C, a polymerase (e.g., RT) is used to extend the 3′ end which was cut by the Cas-gRNA RNP 1150 in a manner such as described with reference to operation C of FIG. 11A and operation B of FIG. 11B, using the portion of the strand that is hybridized to the primer binding site 1101 of gRNA 1100 as a primer, and using the adaptor site 1102 as a template to generate an amplicon that is coupled to the 3′ end which was cut and has a sequence complementary to adaptor site 1102. In operation C of FIG. 11C, the Cas-gRNA RNP and polymerase are removed, exposing the 3′ adaptor (e.g., A14′ and ME′ sequences) in a manner such as described with reference to operation D of FIG. 11A and operation C of FIG. 11B.

At operation D of FIG. 11C, the polynucleotide may be contacted with a transposome (e.g., Tn5 or Tn7) including a 5′ adaptor, and the transposome may cut the polynucleotide and add the adaptor to the cut 5′ end thereof in a manner such as described elsewhere herein. Note that in this example, the transposome activity may be nonspecific and therefore may tagment the polynucleotide at a random position. This operation may be performed simultaneously with, before, or after any of operations A though C. The transposome then may be removed as illustrated in operation E of FIG. 11C, and the resulting fragment may include a first strand that includes 5′ and 3′ adaptors (e.g., B15 and A14′), and a second strand that lacks amplification adaptors although this strand may include a ME′ sequence added by the transposome during tagmentation. The fragment then may be amplified (e.g., using PCR) so as to add sample indexes (i5 and i7 and the complements thereof) and sequencing adaptors (e.g., P5 and P7 adaptors and the complements thereof) as illustrated in operation F of FIG. 11C. During the amplification, fragments including A14 and B15 amplify exponentially. The resulting enriched fragment, having amplification and sequencing adaptors coupled thereto, then may be sequenced to identify target sequence 1110.

FIG. 11D illustrates an alternative example also including operations A, B, and C which may be conducted in the manner described with reference to FIG. 11C. At operation D of FIG. 11D, the polynucleotide may be contacted with a Cas-gRNA RNP/transposase fusion protein such as described with reference to FIGS. 4A-4J or FIGS. 6A-6B. The Cas-gRNA RNP may be deactivated (e.g., may include dCas9 or Cas12k) so as to hybridize to a specific sequence in the polynucleotide, but not cut the polynucleotide. Responsive to the Cas-gRNA RNP of the fusion protein hybridizing to the polynucleotide, the transposase of the fusion protein may tagment the polynucleotide to include a 5′ amplification adaptor. The fluidic and/or biochemical conditions optionally may be controlled in a manner such as described elsewhere herein, so as to inhibit activity of the transposase until after the Cas-gRNA RNP has hybridized to the polynucleotide. Note that in this example, although the transposome activity may be nonspecific, the Cas-gRNA RNP is sequence specific and therefore may tagment the polynucleotide at a position that is selected to flank the target sequence on the other side from that cut during operation B. This operation may be performed simultaneously with, before, or after any of operations A though C of FIG. 11D. The transposome then may be removed as illustrated in operation E of FIG. 11D, and the resulting fragment may include a first strand that includes 5′ and 3′ adaptors (e.g., B15 and A14′), and a second strand that lacks amplification adaptors although this strand may include a ME′ sequence added by the transposome during tagmentation. The fragment then may be amplified (e.g., using PCR) so as to add sample indexes (i5 and i7 and the complements thereof) and sequencing adaptors (e.g., P5 and P7 adaptors and the complements thereof) as illustrated in operation F of FIG. 11D. During the amplification, fragments including A14 and B15 amplify exponentially. The resulting enriched fragment, having amplification and sequencing adaptors coupled thereto, then may be sequenced to identify target sequence 1110.

FIGS. 11E and 11F respectively illustrate fragments that may be generated using the process flows of FIGS. 11C and 11D. As illustrated in FIG. 11C, nonspecific tagmentation may be performed at random locations along the length of the polynucleotide, leading to a range of fragment sizes and a subset of the fragments not including target sequence 1110. In comparison, as illustrated in FIG. 11D, specific tagmentation using a Cas-gRNA RNP/transposase fusion protein may yield fragments of substantially uniform sizes that include the target sequence 1110.

From the foregoing, it will be understood that a variety of different techniques may be used to generate fragments having adaptors suitable for use in amplification and sequencing in a streamlined manner. Method 11000 illustrates a flow of steps in a method. The method may include contacting a Cas-gRNA RNP with a polynucleotide that includes first and second strands (operation 11001). The Cas-gRNA may include a guide RNA including a primer, an amplification adaptor site, and a CRISPR protospacer. The Cas-gRNA also may include a Cas protein binding the CRISPR protospacer. Method 11000 also may include hybridizing the CRISPR protospacer to the first strand (operation 11002). Method 11000 also may include hybridizing the primer to the second strand (operation 11003). Nonlimiting examples of gRNAs, Cas proteins, contact of such Cas-gRNA RNPs with polynucleotides, and hybridization of certain gRNA components to selected regions of the polynucleotides, are provided with reference to FIGS. 11A-11D.

Optionally, method 11000 may include cutting the first and second strands, by the Cas-gRNA RNP, at respective locations based upon the sequence of the CRISPR protospacer, e.g., in a manner such as described with reference to FIGS. 11A-11D. Optionally, method 11000 further may include using a first reverse transcriptase to generate an amplicon of the amplification adaptor site at the cut in the second strand caused by the first Cas protein, e.g., in a manner such as described with reference to FIGS. 11A-11D.

Optionally, method 11000 may include contacting the polynucleotide with a second Cas-gRNA RNP. The second Cas-gRNA RNP may include a second guide RNA that includes a second primer, a second amplification adaptor site, and a second CRISPR protospacer; and a second Cas protein binding the second CRISPR protospacer. Method 11000 may include hybridizing the second CRISPR protospacer to the first strand; and hybridizing the second primer to the second strand. The second Cas-gRNA RNP optionally may cut the first and second at respective locations based upon the sequence of the second CRISPR protospacer. The cuts in the first and second strands by the second Cas-gRNA RNP may be spaced apart from the cuts in the first and second strands by the first Cas-gRNA RNP by at least a target sequence. A second reverse transcriptase may be used to generate an amplicon of the amplification adaptor site at the cut in the second strand caused by the second Cas protein. The first and second Cas-gRNA RNPs and the first and second reverse transcriptases may generate a partially double-stranded polynucleotide fragment having a first end and a second end, the first end comprising a first 3′ overhang; the second end comprising a second 3′ overhang; and a target sequence located between the first and second ends, e.g., in a manner such as described with reference to FIG. 11B. The first 3′ overhang may include the amplicon of the first amplification adaptor site, and the second 3′ overhang may include the amplicon of the second amplification adaptor site. Method 11000 further may include ligating a third amplification adaptor to a 5′ group at the first end; ligating a fourth amplification adaptor to a 5′ group at the second end; amplifying the fragment using the first, second, third, and fourth amplification adaptors; and sequencing the amplified fragment, e.g., in a manner such as described with reference to FIG. 11B.

### Additional Discussion

It will be appreciated that any suitable aspects of the process flows provided herein may be performed in any suitable combination with one another. For example, any suitable operation(s) of method 1000 described with reference to FIG. 1K, any suitable operation(s) of method 2000 described with reference to FIG. 2J, any suitable operation(s) of method 2010 described with reference to FIG. 2K, any suitable operation(s) of method 3000 described with reference to FIG. 3E, any suitable operation(s) of method 4000 described with reference to FIG. 4J, any suitable operation(s) of method 5000 described with reference to FIG. 5K, any suitable operation(s) described with reference to FIGS. 6A-6B, any suitable operation(s) described with reference to FIGS. 7A-7G, any suitable operation(s) of method 8000 described with reference to FIG. 8H, any suitable operation(s) of method 9000 described with reference to FIG. 9F, any suitable operation(s) of method 10000 described with reference to FIG. 10C, and/or any suitable operation(s) of method 11000 described with reference to FIG. 11G. As one purely illustrative example, method 1000 may be used to substantially remove genetic material of one species from a sample, operations from methods 2000, 2010, 3000, 4000, 8000, 9000, 10000, or 11000 may be used to prepare the remaining polynucleotides for sequencing, and operations from method 5000 may be used to perform an epigenetic assay on those polynucleotides. As yet another purely illustrative example, method 1000 may be used to substantially remove genetic material of one species from a sample, and operations from method 5000 may be used to perform an epigenetic assay on the remaining polynucleotides. As still another purely illustrative example, operations from methods 2000, 2010, 3000, 4000, 8000, 9000, 10000, and/or 11000 may be used to prepare polynucleotides for sequencing, and operations from method 5000 may be used to perform an epigenetic assay on those polynucleotides. The results of the epigenetic assay may be compared to the sequence of the polynucleotides.

Accordingly, it may be understood that the present disclosure provides methods for locus-targeted epigenetic identification, that may include providing a composition including a polynucleotide having an epigenetic protein associated therewith; hybridizing the polynucleotide with a first Cas-gRNA RNP and a second Cas-gRNA RNP that specifically hybridize to distinct first target region and a second target regions, respectively, of the polynucleotide and cut the polynucleotide to provide a fragment of the hybridized polynucleotide therebetween, wherein the first and/or second RNP has a label bound thereto; and purifying the hybridized polynucleotide fragment and RNP with a capture element that binds to the label, thereby enriching the composition for the polynucleotide having the epigenetic protein associated therewith.

In some examples, the disclosure further provides removing the RNP from the polynucleotide. In some examples, the disclosure further provides assaying the polynucleotide and the associated epigenetic protein. In some examples, the disclosure provides assaying the polynucleotide and the associated epigenetic protein with a locus-targeted high-multiplex proteome oligo-linked antibody assay, and/or a locus-targeted ATAC-sequencing assay, and/or a ChIP-sequencing assay. In some examples, the disclosure provides a locus specific indication of the epigenetic protein.

In some examples, the disclosure provides locus specific identification of more than one epigenetic protein. In some examples, the disclosure provides hybridizing the polynucleotide more than one pair of a Cas-gRNA RNP and a second Cas-gRNA RNP specifically hybridize to distinct first target regions and a second target regions, respectively, of the polynucleotide and cut the polynucleotide to provide multiple fragments of the hybridized polynucleotide therebetween. In some examples, the first and/or second RNP of each pair of Cas-gRNA RNPs has a label bound thereto for purifying the hybridized polynucleotide fragment and RNP with a capture element that binds to the label, thereby enriching the composition for the polynucleotide having the epigenetic proteins associated therewith.

In some examples, the disclosure provides for the locus specific identification of more than one epigenetic protein on a same chromosome. In some examples, the disclosure provides for hybridizing the pairs of Cas-gRNA RNPs to polynucleotides of the same genome but on different chromosomes. In some examples, the disclosure provides for the locus specific indications for more than one epigenetic protein in a genome.

In some examples, the disclosure provides assaying the polynucleotide and the associated epigenetic protein with a locus-targeted high-multiplex proteome oligo-linked antibody assay, including contacting the polynucleotide and the associated epigenetic protein with an anti-epigenetic protein antibody labeled with an oligonucleotide label corresponding to the epigenetic protein.

In some examples, the disclosure provides for assaying the polynucleotide and the associated epigenetic protein with a locus-targeted ATAC-sequence assay, for example, as described with reference to FIGS. 5I-5J.

Previously known ATAC-sequencing is capable of NGS-based epigenetic studies due to assay simplicity and broad, genome-wide assessment of chromatin accessibility. However, previously ATAC-sequencing is unable to directly identify protein bound at each DNA site, nor deeply resolve binding site and epigenetic changes important for research and clinical markers (e.g., liquid biopsy). Previously known ChIP-sequencing methods directly resolve DNA-binding sites of a particular protein, using methods involving Tn5-proteinA tagmentation directed by antibody bound to the protein of interest. For further details regarding previously known epigenetic assays, see, e.g., the following references, the entire contents of each of which are incorporated by reference herein: Kaya-Okur et al., “CUT&Tag for efficient epigenomic profiling of small samples and single cells,” Nat Comm 10: 1930, 1-10 (2019); Wang et al., “CoBATCH for high-throughput single-cell epigenomic profiling,” Mol Cell 76(1): 206-216.e7 (2019); Ai et al., “Profiling chromatin states using single cell itCHIP-seq,” Nat Cell Biol 21: 1164-1172 (2019); and Carter et al., “Mapping histone modifications in low cell number and single cells using antibody-guided chromatin tagmentation (ACT-seq),” Nat Comm 10: 3747, 1-5 (2019).

In some examples, the disclosure provides enhancing the polynucleotide fragment with exogenous unique molecular identifiers (UMIs), e.g., such as described with reference to FIGS. 3A-3E. In some examples, the disclosure provides generation of targeted sequencing libraries with exogenous UMIs. In some examples, the UMIs are created on the ends of the polynucleotide fragments by targeting multiple Cas nucleases with overlapping DNA-binding footprints to produce diversity in fragment ends, e.g., such as described with reference to FIGS. 4A-4J; in this regard, the diverse fragment ends themselves may be considered to provide UMIs, as distinguished from a separate UMI sequence that may be coupled to the fragment end. It will be appreciated that the diverse fragment ends may be used in conjunction with any suitable sequencing or assay techniques, such as Cas9-mediated negative enrichment, CRISPR-DS, or other dual-Cas9 based CRISPR-targeted LP methods.

In some examples, the disclosure provides Cas9-mediated negative enrichment methods where, from genomic DNA starting material, a Cas-gRNA RNP binds, cleaves and protects the polynucleotide region from exonuclease (III, VII). Alternatively, dCas9 may be used to block exonuclease activity, allowing more flexible sequence targeting, where any dCas9 orientation is allowed as it will not expose targeted region to exonuclease activity. Cas nuclease footprint overlap such as described with reference to FIGS. 4A-4J may ensure that only one Cas nuclease may act on each fragment end. In some examples, the disclosure provides standard ligation-based LP (ER,A-tail, lig) using non-random UMI Y-adapters. In some examples, the disclosure provides using full-length adapters that enables targeted PCR-free. In some examples, the method can also be used without UMIs, relying on non-random unique fragment ends to resolve molecules. This method includes more Cas9 staggering cuts to achieve appropriate fragment end complexity for most assay applications. In some examples, the disclosure provides using a combination of fragment end-coordinates and UMIs to uniquely identify molecules.

In some examples, the disclosure provides Cas-gRNA RNP mediated DNA de-hosting using CRISPR/Cas to cleave host repetitive elements and then degrade them using exonucleases, e.g., in a manner such as described with reference to FIGS. 1A-1J. In some examples, the disclosure provides leveraging programmable nuclease activity of a Cas-gRNA RNP to target repetitive elements that typically that make up >50% of the genomic polynucleotide and are distributed throughout the human genome. In some examples, the disclosure provides using a set of Cas-gRNA RNPs (e.g., between 10 and 1,000,000 Cas-gRNA RNPs) to specifically cleave each human chromosome more than one time. In some examples, the disclosure provides methods for selectively degrading host DNA fragments, while retaining uncleaved non-host/microbial DNA fragments.

In some examples such as described with reference to FIGS. 1A-1K, the disclosure provides a method for Cas-gRNA RNP DNA de-hosting including: (a) modifying DNA in a sample mix to protect ends from exonuclease treatment; (b) cleaving the polynucleotide with Cas-gRNA RNP targeted to host (e.g., human) repeat elements, exposing unprotected host DNA fragment ends; and (c) applying one or more exonucleases to selectively degrade host DNA with the unprotected DNA end. In some examples, in operation (a), to inhibit exonuclease-mediated degradation of linear non-host DNA, the DNA-sample is pre-treated before Cas-gRNA RNP with one or more of the following methods. In some examples, the disclosure provides for inhibiting exonuclease-mediated degradation of linear non-host DNA by ligating an exonuclease-protecting DNA adapter onto the ends of the DNA molecules, such as with a hairpin adapter or a DNA adapter including base modifications resistant to exonuclease activity (for example, phosphorothioate bonds or 3′ phosphate provide protection against many exonuclease activities, including ExoIII). In some examples, the disclosure provides inhibiting exonuclease-mediated degradation of linear non-host DNA by dephosphorylating the DNA fragment 5′ ends to protect against lambda exonuclease activity, which acts 5′-3′ on dsDNA only with 5′ phosphate. In this example, Cas-gRNA RNP cleavage at host DNA sites will expose a 5′ phosphate, the substrate for lambda exonuclease cleavage. In some examples, the disclosure provides for inhibiting exonuclease-mediated degradation of linear non-host DNA by protecting nucleotides with terminal transferase 3′ addition of exonuclease protecting modified nucleotides. In some examples, Taq DNA Polymerase is used to add non-templated nucleotides to dsDNA which incorporates phosphorothioate linkage nucleotides.

In some examples, the disclosure provides a method of uniformly fragmenting genomic DNA, such as for subsequent locus-targeted epigenetic identification, including using Cas-gRNA RNPs nucleases to cleave the DNA at precise positions, controlling the length and uniformity of DNA fragmentation, e.g., such as described with reference to FIGS. 2A-2K. This method may include using duplex sequencing (DS) to resolve unique molecules, and may be employed here for whole genomic DNA analysis. A dual sgRNA pool can be used for host DNA depletion when applied to metagenomic/mixed samples. For example, a Legacy RiboZero-style pull down-load sgRNA pool with biotinylated/tagged Cas9, or a low input compatible ‘DASH’-style depletion Cas9 cleavage of host library molecules post library preparation may be used such as described by Crawford et al., “Depletion of abundant sequences by hybridization (DASH): using Cas9 to remove unwanted high-abundance species in sequencing libraries and molecular counting applications,” Genome Biology 17: 41, 1-13 (2016), the entire contents of which are incorporated by reference herein.

An example method for size controlled whole genome fragmentation by Cas-gRNA RNP cleavage of host library molecules post library preparation is described with reference to FIGS. 2A-2K. The targeted genome fragmentation approach based on multiple Cas-gRNA RNP digestion produces DNA fragments of similar length. These fragments can be enriched by a simple size selection, resulting in targeted enrichment. Additionally, homogenous length fragments may significantly reduce PCR amplification bias and may enhance read usability. The disclosure provides target enrichment with duplex sequencing, using double-strand molecular tagging to correct for sequencing errors. The CRISPR-DS technique enables efficient target enrichment of small genomic regions, even coverage, ultra-accurate sequencing, and reduced DNA input. In some examples, the disclosure provides that in association with the UMI approach to generate DNA fragment end diversity by targeting multiple Cas-gRNA RNPs to a targeted region, this CRISPR-DS targeting approach can be utilized to increase resolvable library complexity with a given number of UMIs, and increase sequencing coverage of individual Cas cut sites.

Cas-gRNA RNP cleavage is known to yield predominantly blunt ends, but also small overhangs. Exonuclease activity during the end-repair operation of library preparation may lead to loss of sequence information at/near the cleavage site. In some examples, staggering cleavage sites at a target with multiple guide RNAs, e.g., in a manner such as described with reference to FIGS. 3A-3E may reduce local coverage losses. Note that because of the high sequence specificity of the Cas-gRNA RNP targeting, the identity of bases at or near the cut site are inferable with confidence.

In some examples, the methods provided herein includes applying at least one transposase, and at least one transposon end composition including an oligonucleotide, to a sample including a target polynucleotide under conditions where the target polynucleotide and the transposon end composition undergo a transposition reaction to generate a mixture, wherein the target polynucleotide is fragmented to generate a plurality of target polynucleotide fragments, and thus incorporates an oligonucleotide sequence into each of the plurality of target polynucleotide fragments.

### Additional Comments

The practice of the present disclosure may employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

All publications, patents, and patent applications mentioned in this specification are herein incorporated by reference to the same extent as if each individual publication, patent, or patent application was specifically and individually indicated to be incorporated by reference.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of treating a mixture of first double-stranded polynucleotides from a first species and second double-stranded polynucleotides from a second species, the method comprising:
protecting ends of the first double-stranded polynucleotides and any ends of the second double-stranded polynucleotides;
after protecting the ends of the first and second double-stranded polynucleotides, selectively generating free ends within the first double-stranded polynucleotides; and
degrading the first double-stranded polynucleotides from the free ends toward the protected ends.

2. The method of claim 1, wherein selectively generating the free ends within the first double-stranded polynucleotides comprises hybridizing CRISPR-associated protein guide RNA ribonucleoproteins (Cas-gRNA RNPs) to sequences that are present within the first double-stranded polynucleotides and that are not present within the second double-stranded polynucleotides, and cutting the sequences with the Cas-gRNA RNPs.

3. The method of claim 2, wherein the sequences comprise mammalian specific repetitive elements or human specific repetitive elements.

4. (canceled)

5. The method of claim 1, wherein the first double-stranded nucleotides comprise a plurality of chromosomes from the first species.

6. The method of claim 1, wherein the second species is bacterial, fungal, or viral.

7. The method of claim 1, wherein protecting ends of the first and second double-stranded polynucleotides comprises at least one of: ligating hairpin adapters to the ends, 5′-dephosphorylating the ends, or adding modified bases to the ends.

8-9. (canceled)

10. The method of claim 7, wherein the modified bases comprise phosphorothioate bonds or wherein the modified bases are added using a terminal transferase.

11. (canceled)

12. The method of claim 1, wherein degrading the first double-stranded polynucleotides is performed using an exonuclease.

13. The method of claim 1, wherein the free ends include 3′ ends, or wherein the free ends include 5′ ends.

14. The method of claim 1, wherein degrading the first double-stranded polynucleotides is performed using exonuclease III or using Lambda exonuclease.

15-16. (canceled)

17. The method of claim 1, further comprising subsequently ligating amplification adapters to the ends of any remaining double-stranded polynucleotides in the mixture.

18. The method of claim 17, wherein the amplification adapters include unique molecular identifiers (UMIs).

19. The method of claim 17, further comprising subsequently amplifying and sequencing the double-stranded polynucleotides.

20. The method of claim 1, wherein the first double-stranded polynucleotides comprise double-stranded DNA, or wherein the second double-stranded polynucleotides comprise double-stranded DNA, or wherein the second double-stranded polynucleotides comprise circular DNA.

21-22. (canceled)

23. The method of claim 1, wherein the Cas comprises Cas9.

24. A composition, comprising:
first double-stranded polynucleotides from a first species, wherein ends of the first double-stranded polynucleotides are protected;
second double-stranded polynucleotides from a second species, wherein any ends of the second double-stranded polynucleotides are protected; and
CRISPR-associated protein guide RNA ribonucleoproteins (Cas-gRNA RNPs) hybridized to sequences that are present within the first double-stranded polynucleotides and that are not present within the second double-stranded polynucleotides, the Cas-gRNA RNPs being for cutting the sequences so as to selectively generate free ends within the first double-stranded polynucleotides.

25. The composition of claim 24, wherein the sequences comprise mammalian specific repetitive elements or human specific repetitive elements.

26. (canceled)

27. The composition of claim 24, wherein the second species is bacterial, fungal, or viral.

28. The composition of claim 24, wherein the ends of the first and second double-stranded polynucleotides are protected using at least one of: hairpin adapters, 5′-dephosphorylation, or modified bases.

29-30. (canceled)

31. The composition of claim 28, wherein the modified bases comprise phosphorothioate bonds.

32. The composition of claim 24, wherein the free ends include 3′ ends, or wherein the free ends include 5′ ends.

33. (canceled)

34. The composition of claim 24, wherein the first double-stranded polynucleotides comprise double-stranded DNA, or wherein the second double-stranded polynucleotides comprise double-stranded DNA, or wherein the second double-stranded polynucleotides comprise circular DNA.

35-36. (canceled)

37. The composition of claim 24, wherein the Cas comprises Cas9.

38-443. (canceled)


SEQUENCE LISTING (4 sequences)

SEQ ID NO: 1 - DNA, 29 bp, Artificial Sequence
  Primer
aatgatacggcgaccaccgagauctacac

SEQ ID NO: 2 - DNA, 25 bp, Artificial Sequence
  Primer
  misc_feature (23)..(23): n = G or 8-oxoguanine
caagcagaagacggcatacgagnat

SEQ ID NO: 3 - DNA, 20 bp, Artificial Sequence
  Primer
aatgatacggcgaccaccga

SEQ ID NO: 4 - DNA, 21 bp, Artificial Sequence
  Primer
caagcagaagacggcatacga